SEQUENCE LISTING (59 sequences)

SEQ ID NO: 1 - protein, 950 aa, Trichderma Reesei
MAAVGPLAMHPTVTEHDYRFPRRPDPHRSTDRSFARMDSRDDHVALANGNASAAVTSGQAASSRKNSVLDSALFPSLENAASGSSSSLEQMQSDDPLAAQIWKFFNRTKQQLPNQQRMENLTWRMMALNLRKRQQQQQQQKTHNRSLSQPSQPQNAPSGIAQQLRKTSDSHAEGVDAMNIDDFIFAEEGGASVKDNSPQPPASSLSGPQTAATKTSKSSRPLASAIPIKGRKGSSANQFVAQSVPVAPPHQRGQFDEFHYVPRHDRKTSIDDRRTRKRPANFSPHVSAINPSASGLSLDLEADPDLNGYSLDNTNAMSLQQQMANNQGSAFNLDFMDSDGVLGSAPFHPNFSFSPSTSPMMHNGPFPSVYNNAALQSSLNNPDFYSPTGSGYPSATSTPHPMNEGDGVYFGSQEMQHHPHHRAQGFRQSSGNLNHHLSHQFMYGDSNGGNGSGGGNGAAFPSTGASSEAVASYSAAQNSFGHIDPSQVFQTESQVASPVLPMQQGNMLGFGGESDEDDGTAFLDRGGVGLHGDFTSAVDEAGTFSWDASLPGQFSTQAARFPGGPTRKHVQIGTITTDYVDTSNDWDGHNLSRSHSQTLGMGNDKRQNKLPRNSSTPSHMAAKHSGFEQIAQSLPSSPGPHASGTASGFSSVAPSRPASPPPSSKHGSSTNLHAASGNPGENSAPTTCTNCFTQTTPLWRRNPEGQPLCNACGLFLKLHGVVRPLSLKTDVIKKRNRGSGANLPVGGSRKKNAGNGSNAASRKNSSLSMAAAAAATAAVATANHQAATPPSTTRPSSNKEGDSPASSGHHTAGSTPGSQYGGATIVGGKGVVPIAAAPPKTTPGPGASSSMSRPTTASSKRQRRHSKSEGTSSSAMDLDGPGDSIGRQLGATPSMPSISSTMLSNGSFTMGQRSMMGSSMVSMGGSQGNLVNSAGGSSGPQEWEWLTMSL

SEQ ID NO: 2 - DNA, 3373 bp, Trichoderma reesei
atggcagctgtcggaccgcttgcgatgcacccgaccgtcacagaacacgattaccgcttcccgcgaagacccgatcctcatcgaagtaccgaccgcagtttcgcccgcatggattcgcgagacgaccatgtcgcactcgccaacggcaatgcctccgccgccgtgacgagcgggcaagccgcctcgtcgcgcaagaacagcgtcctcgactccgccttgtttccatcgctcgaaaacgccgcctccggctcgtccagcagcctcgagcagatgcagagcgacgatccgctggcggcccagatctggaagttcttcaacaggacgaagcagcagctgccgaaccagcagcgaatggagaacctgacgtggcgcatgatggccctcaacctgcgcaagcgtcagcagcagcaacagcagcagaagacgcacaacaggtattattatagcaaatctcctttgccatccgtctcatgcttcgccatccccatctgattggtctgccgttctttgcatcgatgcccgtctaaaaggctagatacggcttggctttccctttgggggggaggggagcgggttcgcccttcatcacatcatcccgccgctggctgacatgtttttctgatctcgtctttccctgcatcacagatccctctcgcaaccaagtcaaccacaaaacgctcccagcggcatcgcccagcagctgcgaaagacatccgatagccatgctgagggcgtggatgccatgaacattgacgacttcatcttcgccgaggagggaggagcctccgtcaaggacaactcaccgcagccgcccgcatcctctctctctggtccgcagaccgctgccaccaagaccagcaaatcatccaggccgctggcttcggcaatccccatcaagggccgcaagggttcctctgccaatcaattcgtcgcccagtccgtccctgtagcgccacctcaccagcggggacagtttgatgaattccattacgtccctcgacacgatagaaagacgagtatagacgaccgccgggtgagtgcattgatttttgattggaacagaagcccctacccagcggatcaagcctcccaatgccctcccctcctccctcgcagccatcttctcctctattttatacgtttcttgacggcttgtcgtgcatggcacgccacgttccgccttcgcattctgcctgcttatccttcctccttaacttacatgcactccgtccctctgccctttaaataccctagccgtcgttcctgtgtccgaggacatctgcccagttctaatttcgctcttcccgtggactatgtgaggttgctaacaatgtctcccaagacgaggaaacggcctgccaacttctcaccccatgtgagcgccatcaacccgagtgcgtctggtctcagcctcgatctggaagcagacccggatctcaatggatactccctcgacaacacaaacgcgatgtccctgcagcagcaaatggccaacaaccagggctctgctttcaacctggacttcatggacagcgacggcgtccttggctctgcccccttccatccgaacttctccttttctccatcgacgtctccaatgatgcacaatggaccgtttccatccgtgtacaacaacgccgcgctgcaatcttcgctgaacaaccccgacttctactctcccactggctcggggtatccatcggccacatccaccccacatccgatgaacgagggcgacggcgtctatttcggctcccaggaaatgcagcaccatccgcatcatcgtgcacagggcttccgacaaagctccggtaacctcaaccaccacttgagccaccagttcatgtacggcgattccaatggcggcaacggcagcggcggcggcaatggcgctgccttcccctccacaggcgccagctcggaggcggtggcatcgtacagcgctgctcaaaactcttttgggcacatcgacccaagccaagtgttccagacggagtcgcaggtcgcctctcccgtcttaccgatgcagcaaggcaacatgcttggtttcggaggagagtctgacgaagacgacggcaccgccttcttggatcgcggtggcgttggcctccacggtgacttcacatctgccgtcgacgaggcagggacgttcagctgggacgcctctttgccgggacagttcagcacgcaggctgctcgctttcccggcggccccactcgcaagcacgtacagattgggaccatcacgactgattacgtcgacaccagtaacgactgggatggccataacctgagccgatctcactcgcaaacgctcggcatgggcaacgacaagcgacagaacaaacttcctcgcaactcgtcgacaccctcgcacatggccgccaagcacagtggattcgagcaaatcgcccagtctctccccagctcaccgggtccccatgcttcgggaaccgcctccggcttctcgtcagttgcgccaagcagaccagcctcaccgcccccaagctccaagcacggctcctcgaccaacctgcatgcggctagcggaaaccccggagagaacagcgctcccactacctgcaccaactgcttcacccagacgactcctctgtggagacggaaccccgagggccagcccctctgcaatgcctgtggcctcttcctcaagctacacggcgtcgtgcgtcccctcagtctgaagacggacgttatcaagaagagaaacaggggctccggtgccaacctcccggttggaggatcgaggaagaagaacgcaggcaatggctccaacgccgcctcgaggaagaactcatcgctgtccatggctgccgctgccgcggcgacggcagctgttgctaccgcaaaccaccaagccgcgacgccgccatctacaactcgcccaagcagcaataaggagggcgatagccccgccagcagtggccatcacaccgcaggcagcacgcctggcagccaatatggaggagccaccattgtgggcggcaaaggcgtcgtgcccattgcggcggcaccacccaagacaacgcctggacccggagcctcgtcgtccatgtcccgaccgaccacggcgtcctccaaaagacagcgtaggcatagcaagagcgagggcacttcatcgtcggctatggatctcgacggccctggagactctattgggcgacagcttggcgcaactcccagcatgccttccatctctagcactatgctttcgaatggaagcttcaccatgggtcagcgctccatgatgggatcaagcatggtgtccatgggtggttcccagggcaaccttgtgaacagcgccggtggctcgtcaggccctcaggaatgggagtggttgactatgagtctttga

SEQ ID NO: 3 - DNA, 2853 bp, Trichoderma reesei
atggcagctgtcggaccgcttgcgatgcacccgaccgtcacagaacacgattaccgcttcccgcgaagacccgatcctcatcgaagtaccgaccgcagtttcgcccgcatggattcgcgagacgaccatgtcgcactcgccaacggcaatgcctccgccgccgtgacgagcgggcaagccgcctcgtcgcgcaagaacagcgtcctcgactccgccttgtttccatcgctcgaaaacgccgcctccggctcgtccagcagcctcgagcagatgcagagcgacgatccgctggcggcccagatctggaagttcttcaacaggacgaagcagcagctgccgaaccagcagcgaatggagaacctgacgtggcgcatgatggccctcaacctgcgcaagcgtcagcagcagcaacagcagcagaagacgcacaacagatccctctcgcaaccaagtcaaccacaaaacgctcccagcggcatcgcccagcagctgcgaaagacatccgatagccatgctgagggcgtggatgccatgaacattgacgacttcatcttcgccgaggagggaggagcctccgtcaaggacaactcaccgcagccgcccgcatcctctctctctggtccgcagaccgctgccaccaagaccagcaaatcatccaggccgctggcttcggcaatccccatcaagggccgcaagggttcctctgccaatcaattcgtcgcccagtccgtccctgtagcgccacctcaccagcggggacagtttgatgaattccattacgtccctcgacacgatagaaagacgagtatagacgaccgccggacgaggaaacggcctgccaacttctcaccccatgtgagcgccatcaacccgagtgcgtctggtctcagcctcgatctggaagcagacccggatctcaatggatactccctcgacaacacaaacgcgatgtccctgcagcagcaaatggccaacaaccagggctctgctttcaacctggacttcatggacagcgacggcgtccttggctctgcccccttccatccgaacttctccttttctccatcgacgtctccaatgatgcacaatggaccgtttccatccgtgtacaacaacgccgcgctgcaatcttcgctgaacaaccccgacttctactctcccactggctcggggtatccatcggccacatccaccccacatccgatgaacgagggcgacggcgtctatttcggctcccaggaaatgcagcaccatccgcatcatcgtgcacagggcttccgacaaagctccggtaacctcaaccaccacttgagccaccagttcatgtacggcgattccaatggcggcaacggcagcggcggcggcaatggcgctgccttcccctccacaggcgccagctcggaggcggtggcatcgtacagcgctgctcaaaactcttttgggcacatcgacccaagccaagtgttccagacggagtcgcaggtcgcctctcccgtcttaccgatgcagcaaggcaacatgcttggtttcggaggagagtctgacgaagacgacggcaccgccttcttggatcgcggtggcgttggcctccacggtgacttcacatctgccgtcgacgaggcagggacgttcagctgggacgcctctttgccgggacagttcagcacgcaggctgctcgctttcccggcggccccactcgcaagcacgtacagattgggaccatcacgactgattacgtcgacaccagtaacgactgggatggccataacctgagccgatctcactcgcaaacgctcggcatgggcaacgacaagcgacagaacaaacttcctcgcaactcgtcgacaccctcgcacatggccgccaagcacagtggattcgagcaaatcgcccagtctctccccagctcaccgggtccccatgcttcgggaaccgcctccggcttctcgtcagttgcgccaagcagaccagcctcaccgcccccaagctccaagcacggctcctcgaccaacctgcatgcggctagcggaaaccccggagagaacagcgctcccactacctgcaccaactgcttcacccagacgactcctctgtggagacggaaccccgagggccagcccctctgcaatgcctgtggcctcttcctcaagctacacggcgtcgtgcgtcccctcagtctgaagacggacgttatcaagaagagaaacaggggctccggtgccaacctcccggttggaggatcgaggaagaagaacgcaggcaatggctccaacgccgcctcgaggaagaactcatcgctgtccatggctgccgctgccgcggcgacggcagctgttgctaccgcaaaccaccaagccgcgacgccgccatctacaactcgcccaagcagcaataaggagggcgatagccccgccagcagtggccatcacaccgcaggcagcacgcctggcagccaatatggaggagccaccattgtgggcggcaaaggcgtcgtgcccattgcggcggcaccacccaagacaacgcctggacccggagcctcgtcgtccatgtcccgaccgaccacggcgtcctccaaaagacagcgtaggcatagcaagagcgagggcacttcatcgtcggctatggatctcgacggccctggagactctattgggcgacagcttggcgcaactcccagcatgccttccatctctagcactatgctttcgaatggaagcttcaccatgggtcagcgctccatgatgggatcaagcatggtgtccatgggtggttcccagggcaaccttgtgaacagcgccggtggctcgtcaggccctcaggaatgggagtggttgactatgagtctttga

SEQ ID NO: 4 - DNA, 43 bp, artificial sequence
  primer
ctcgagtttttcagcaagatactagtcctgaccttatttcccg

SEQ ID NO: 5 - DNA, 30 bp, artificial sequence
  primer
acagctgcagctgcggtcggtacttcgatg

SEQ ID NO: 6 - DNA, 28 bp, artificial sequence
  primer
ccgaccgcagctgcagctgtggagccgc

SEQ ID NO: 7 - DNA, 33 bp, artificial sequence
  primer
tacgctgtctcatgacacatgcatccaccatcg

SEQ ID NO: 8 - DNA, 28 bp, artificial sequence
  primer
atgtgtcatgagacagcgtaggcatagc

SEQ ID NO: 9 - DNA, 42 bp, artificial sequence
  primer
aggagatcttctagaaagatactagtggaaagaaacgtaaag

SEQ ID NO: 10 - DNA, 26 bp, artificial sequence
  primer
gatgaaagtgggatgaatgacaggga

SEQ ID NO: 11 - DNA, 24 bp, artificial sequence
  primer
attcggcgtgcacatcaaaggcgt

SEQ ID NO: 12 - DNA, 25 bp, artificial sequence
  primer
ccgcttaattttcgcccttttttca

SEQ ID NO: 13 - DNA, 19 bp, artificial sequence
  primer
ctagcgcacgaaagacgcg

SEQ ID NO: 14 - DNA, 21 bp, artificial sequence
  primer
agaagaagatgttggcgacct

SEQ ID NO: 15 - DNA, 25 bp, artificial sequence
  primer
tcttgaccaactctatcagagcttg

SEQ ID NO: 16 - DNA, 21 bp, artificial sequence
  primer
tcccagcatgccttccatctc

SEQ ID NO: 17 - DNA, 26 bp, artificial sequence
  primer
gagaatattgtaggagatcttctaga

SEQ ID NO: 18 - DNA, 25 bp, artificial sequence
  primer
actagtcctgaccttatttcccgca

SEQ ID NO: 19 - DNA, 28 bp, artificial sequence
  primer
actagtggaaagaaacgtaaagtatctg

SEQ ID NO: 20 - DNA, 23 bp, artificial sequence
  primer
tttttctcgcctttgcgctgact

SEQ ID NO: 21 - DNA, 23 bp, artificial sequence
  primer
cgtcgcggtgagttcaggctttt

SEQ ID NO: 22 - DNA, 23 bp, artificial sequence
  primer
cgtcgcggtgagttcaggctttt

SEQ ID NO: 23 - DNA, 23 bp, artificial sequence
  primer
cgtcgcggtgagttcaggctttt

SEQ ID NO: 24 - DNA, 22 bp, artificial sequence
  primer
cactcgtccgagggcaaaggaa

SEQ ID NO: 25 - DNA, 22 bp, artificial sequence
  primer
actgaagaggggcctaagaaag

SEQ ID NO: 26 - DNA, 22 bp, artificial sequence
  primer
cactcgtccgagggcaaaggaa

SEQ ID NO: 27 - DNA, 22 bp, artificial sequence
  primer
cactcgtccgagggcaaaggaa

SEQ ID NO: 28 - protein, 1037 aa, Myceliophthora heterothallica
MAATTTDSSAAPGPLAPMHPTTTEHDFRFPRRPPADAATGARKTDRLESASGTKPAPPTSTSTATPRTATAGTTTATAGADGLRPGLLDGRLNLGLAQSGGAAHLDLLRVSAFPPFQQSARREAQSLEEMQREDPLATQIWKFYAKTKQLLPAQERMENLTWRMMHLKLQKTKAANTTKTSRASSSSANTPSGIAQLRKSSEHAPPQLDPMNLDDFIHNDNVGTLASLALTPTPETMRQAEERSAHTTATAIPIKNRKEPSSSSSSSSSSQHLAPQSVPVAAHQRPRDEFGYLPRHPRKTSIDETGQRVSSLTRKRPANFSPHVPAVNSGLSTSGLDADADLQEYSLDHLQHPAAASQPPNQMGVPFPLDTFRLGNDLSISSAGPFQQNFSFSPATSPMAANDPFASLYNSSSMPSGSLGADFYSPPGSAYQSAVSTPHPLGEADGFYFGSMDVRQMRQHPYRNGPAAMANPLGQQFPYGANGSLMFPAASTGPETSSAFTAPSSFSHVDPTRVFQQELPARSPAVGLGQESMFSFGADSDDEDGGAFADRNLPISQDLSPQGSSMDDGAFDSSSLQWDPSLPGNFSMQAARYPAGPTRKQVTIGGTTTDYVDSKGDWEGGSLNRSQSQPFRTTNGRVGKVPRTASTPRLTSKGNPFDRLAQSTPNSPPAEANQNSAFSSVSTSRPSSPPPASRQGSTTNLQAAAGSSQNESSSSGGGGGSSGSTNPNSNASNNNNNNNNNNNNNSSSNTNTPTTCTNCFTQTTPLWRRNPEGQPLCNACGLFLKLHGVVRPLSLKTDVIKKRNRGSGSSLPVGGGGTRSRKNVGSSGGISKRGSTLSVSSSANPPPAQASTPPAASNRTGSANDSESSANGAASTGNGNTPTGYHGNATSSTNVAVGGKGVVPIAAAPAKNLPGPGAASIPRTTAISSSSLSSSKRQRRHSKSVTAAEQPSLNSMDIDSPESSTGSNEGAATSSAGLSKPSLGSSAGLASLQPSASSFSLGGFGSSGGSNGMQRPLAGPGSQRPAPQEWEWLTMSL

SEQ ID NO: 29 - DNA, 3447 bp, Myceliophthora heterothallica
atggcagcgaccacgacggattcctctgcggcgcctggtccgctggccccaatgcatcccaccacgacagagcacgactttcgctttccgcgaaggccgccggcagacgccgcgaccggggcgcggaaaaccgaccgactcgaatctgcctccggtaccaagccggcgccgccgacatcaacgtcgacggcgaccccgaggacggcgacggcggggaccacgacggcgacggcgggcgccgacggattgcgtcccggcctgctcgatggccgcctgaacctaggcctggctcagagtggcggcgccgcccaccttgacctgctgcgcgtctccgccttcccgccgttccagcagagtgcccggcgagaggcccagagcctggaggagatgcagagggaagacccgctggccacccagatctggaagttttacgcaaagacaaaacagctcctgcccgcccaggagcgcatggaaaacctgacgtggcggatgatgcatctcaagttacagaagacgaaggcagccaacacgacaaagtaggtgttttttttttctctttctttctttcccgtcttggtgtcccgcggattgtgtggtacttcgccggttggaggactgggagaaagaaagcaaaaaagaaagaaaaaaaaaagagagaaagaaagcttgggagagagccgggtagaaaaagaagaatgcttgctgactcctgtgggcaaacaaagaacaagtcgtgcatccagctcatcagcaaatacccccagtgggattgcgcaattgcgcaagtcgtcggaacatgccccgccacagttggacccgatgaacctggacgatttcattcacaacgacaacgtcggaaccctggccagcctcgccttgacgccgaccccagagacgatgcgccaggcggaggagagatcggctcacaccacggcgacggccattcccatcaagaaccgcaaggagccgtcgtcgtcgtcatcgtcatcgtcgtcgtctcagcatctcgcaccccagtcggtgcccgttgccgcccaccagaggcctcgggacgaatttggctatctgcctcggcacccgcgcaaaactagcattgacgagaccggccaacgtgtaagttctctcgtctttttgctgctgcgtacgggtcgtctttggagtcaattcatcgggcgccccttgaagccgtcttgtattttcgatgacacgagatgccttcctgcttttttttgtgacggcggtctgatgatccattgcctctctctctagacccgcaaacgaccggccaatttctccccccacgtacccgccgtcaacagcggcctgtcgacaagtggactcgatgccgacgctgacctccaggaatactcactcgaccacttacaacatcccgccgccgccagccaaccgcccaatcagatgggggttcccttcccgctcgacaccttccgcttggggaacgacctgagcatcagctctgccgggcccttccagcagaacttctccttctcgccggccacctcgcccatggcggcaaatgacccgttcgccagcctctataatagctcctcgatgccttcgggctccctaggggccgacttctattcccctcccggctcggcctatcagtccgccgtttccaccccgcatcccctgggcgaggccgatggcttctactttggctccatggatgtgcggcagatgcgccaacatccgtataggaatggccctgccgccatggccaacccgctaggccaacaattcccctacggcgcgaatgggagcctgatgttcccggccgcatcgaccggccccgagacgtcctcggcttttaccgcgccgagctctttcagccatgtcgacccgacgcgggtgttccagcaagagctccctgctcgttcccccgccgtcgggctgggccaagagagcatgttctccttcggagccgattcggacgacgaggacggcggcgcctttgccgaccgcaatctgccaatctcgcaggacctctcgccccaaggaagcagtatggatgacggtgcgtttgacagttcgtcactgcagtgggatccatccctcccgggcaacttcagcatgcaggcggccaggtatccggccgggcctactcggaaacaggtgacaataggcggcaccacgaccgactacgtcgactcgaagggggattgggagggcggaagtctgaacaggtcgcagtcgcagcctttccgtacaacgaacggtcgcgtcgggaaggtaccgcggacagcatcgaccccgaggttgacaagcaagggcaatccgttcgacaggttggcacagtcgaccccgaactcgccgccagccgaggccaaccagaactcggccttctcctctgtctctacaagccgcccgtcctcgcctcctccggcttcgaggcagggttccacaaccaacctccaagccgccgcgggaagcagtcagaacgagagcagcagcagcggcggcggcggcggcagcagcggcagcacgaaccccaactccaacgccagcaacaacaacaacaataacaataacaataacaacaacaacagttcctccaacactaacacccccacaacatgtaccaactgcttcacccagacaacgccgctctggcgccgcaatcccgaaggccaacccctctgcaacgcttgcggtcttttcctcaagctgcatggtgtggttcgcccgctgagcttgaagacggacgtcatcaagaagcggaaccgcggctccggttcgagtcttcctgtcggcggcggcggcacccggtcgaggaagaacgtcggctcttctggggggataagcaagaggggctcgactttatccgtctcctccagcgccaaccctcccccagctcaggcgagcaccccgccggcagcctcgaaccggaccggtagcgccaacgacagcgaaagttccgcgaacggcgccgcgtcgaccgggaatggaaacacgccaaccggctaccacggcaatgccacctcctctaccaacgttgccgttggcggcaagggtgtcgttccgatcgccgcggccccggcgaagaaccttcccggcccgggcgccgcctccataccgcgcaccacagccatctcctcttcctcgttgtcttcgtcgaaacgccaacgccgccacagcaagagcgtcacagcggcagagcagccgtcgctgaacagcatggacatcgacagcccggagagctctacgggatccaacgagggcgcggcaacatcgtcggccggcctgtcgaagccgagcctgggttcgagcgcggggctggcttcgttgcagcccagtgctagcagctttagtttgggtggctttggcagtagcggcggcagcaacggcatgcagaggccattggccgggccaggaagccagcggcctgcgccacaggagtgggagtggttgaccatgagtttatag

SEQ ID NO: 30 - DNA, 3114 bp, Myceliophthora heterothallica
atggcagcgaccacgacggattcctctgcggcgcctggtccgctggccccaatgcatcccaccacgacagagcacgactttcgctttccgcgaaggccgccggcagacgccgcgaccggggcgcggaaaaccgaccgactcgaatctgcctccggtaccaagccggcgccgccgacatcaacgtcgacggcgaccccgaggacggcgacggcggggaccacgacggcgacggcgggcgccgacggattgcgtcccggcctgctcgatggccgcctgaacctaggcctggctcagagtggcggcgccgcccaccttgacctgctgcgcgtctccgccttcccgccgttccagcagagtgcccggcgagaggcccagagcctggaggagatgcagagggaagacccgctggccacccagatctggaagttttacgcaaagacaaaacagctcctgcccgcccaggagcgcatggaaaacctgacgtggcggatgatgcatctcaagttacagaagacgaaggcagccaacacgacaaaaacaagtcgtgcatccagctcatcagcaaatacccccagtgggattgcgcaattgcgcaagtcgtcggaacatgccccgccacagttggacccgatgaacctggacgatttcattcacaacgacaacgtcggaaccctggccagcctcgccttgacgccgaccccagagacgatgcgccaggcggaggagagatcggctcacaccacggcgacggccattcccatcaagaaccgcaaggagccgtcgtcgtcgtcatcgtcatcgtcgtcgtctcagcatctcgcaccccagtcggtgcccgttgccgcccaccagaggcctcgggacgaatttggctatctgcctcggcacccgcgcaaaactagcattgacgagaccggccaacgtgtaagttctctcacccgcaaacgaccggccaatttctccccccacgtacccgccgtcaacagcggcctgtcgacaagtggactcgatgccgacgctgacctccaggaatactcactcgaccacttacaacatcccgccgccgccagccaaccgcccaatcagatgggggttcccttcccgctcgacaccttccgcttggggaacgacctgagcatcagctctgccgggcccttccagcagaacttctccttctcgccggccacctcgcccatggcggcaaatgacccgttcgccagcctctataatagctcctcgatgccttcgggctccctaggggccgacttctattcccctcccggctcggcctatcagtccgccgtttccaccccgcatcccctgggcgaggccgatggcttctactttggctccatggatgtgcggcagatgcgccaacatccgtataggaatggccctgccgccatggccaacccgctaggccaacaattcccctacggcgcgaatgggagcctgatgttcccggccgcatcgaccggccccgagacgtcctcggcttttaccgcgccgagctctttcagccatgtcgacccgacgcgggtgttccagcaagagctccctgctcgttcccccgccgtcgggctgggccaagagagcatgttctccttcggagccgattcggacgacgaggacggcggcgcctttgccgaccgcaatctgccaatctcgcaggacctctcgccccaaggaagcagtatggatgacggtgcgtttgacagttcgtcactgcagtgggatccatccctcccgggcaacttcagcatgcaggcggccaggtatccggccgggcctactcggaaacaggtgacaataggcggcaccacgaccgactacgtcgactcgaagggggattgggagggcggaagtctgaacaggtcgcagtcgcagcctttccgtacaacgaacggtcgcgtcgggaaggtaccgcggacagcatcgaccccgaggttgacaagcaagggcaatccgttcgacaggttggcacagtcgaccccgaactcgccgccagccgaggccaaccagaactcggccttctcctctgtctctacaagccgcccgtcctcgcctcctccggcttcgaggcagggttccacaaccaacctccaagccgccgcgggaagcagtcagaacgagagcagcagcagcggcggcggcggcggcagcagcggcagcacgaaccccaactccaacgccagcaacaacaacaacaataacaataacaataacaacaacaacagttcctccaacactaacacccccacaacatgtaccaactgcttcacccagacaacgccgctctggcgccgcaatcccgaaggccaacccctctgcaacgcttgcggtcttttcctcaagctgcatggtgtggttcgcccgctgagcttgaagacggacgtcatcaagaagcggaaccgcggctccggttcgagtcttcctgtcggcggcggcggcacccggtcgaggaagaacgtcggctcttctggggggataagcaagaggggctcgactttatccgtctcctccagcgccaaccctcccccagctcaggcgagcaccccgccggcagcctcgaaccggaccggtagcgccaacgacagcgaaagttccgcgaacggcgccgcgtcgaccgggaatggaaacacgccaaccggctaccacggcaatgccacctcctctaccaacgttgccgttggcggcaagggtgtcgttccgatcgccgcggccccggcgaagaaccttcccggcccgggcgccgcctccataccgcgcaccacagccatctcctcttcctcgttgtcttcgtcgaaacgccaacgccgccacagcaagagcgtcacagcggcagagcagccgtcgctgaacagcatggacatcgacagcccggagagctctacgggatccaacgagggcgcggcaacatcgtcggccggcctgtcgaagccgagcctgggttcgagcgcggggctggcttcgttgcagcccagtgctagcagctttagtttgggtggctttggcagtagcggcggcagcaacggcatgcagaggccattggccgggccaggaagccagcggcctgcgccacaggagtgggagtggttgaccatgagtttatag

SEQ ID NO: 31 - protein, 51 aa, Artificial Sequence
  Zinc Finger
PTTCTNCFTQTTPLWRRNPEGQPLCNACGLFLKLHGVVRPLSLKTDVIKKR

SEQ ID NO: 32 - DNA, 6 bp, Artificial Sequence
  Zinc Finger binding site
wgatar

SEQ ID NO: 33 - protein, 1021 aa, Myceliophthora thermophila
MAATTTDSSAAPGPLAPMHPTMTEHDFRFPRRPADAATGARKTDRLESASGTKPVPPTSTSTAASRTATSGTTTAAAGGGGLRPGLLDGRLNLGLAQGGGAAHLDLLRASAFPPFQQSARREAQSLEEMQREDPLATQIWKFYAKTKQLLPAQERMENLTWRMMHLKLQKTKAANTTKTSRASSSSANTPSGIAQLRKSSEHAPPQLDPMNLDDFIHNDNVGTPAGLALTPTPETMRQAEERSAHTTATAIPIKNRKEPSSSSSSSSSQHLAPQSVPVAAHQRPQDEFGYLPRHPRKTSIDETGQRTRKRPANFSPHVPAVNSGLSTSGLDADADLQEYSLDHLQHPAAASQPPNQMGVPFPLDTFRLGNDLSISSAGPFQQNFSFSPATSPMAANDPFASLYNASSMPSGSLGADFYSPPGSAYQSAVSTPHPLGEADGFYFGSMDVRQMRQHPYRNGPAAMANPLGQQFPYGANGGNLMFPAASTGPETSSAFTAPSSFSHVDPTRVFQQDLPARSPAVGLGQESMFSFGADSDDEDGGAFADRNLPISQDLSPQGSSMDDGAFDCSSLQWDPSLPGNFSMQAARYPGGPTRKQVTIGGTTTDYVDSKGDWEGGNLNRSQSQPFRTTNGRVGKVPRTASTPRLTSKGNPFDRLAQSTPNSPPAEANQNSAFSSVSTSRPSSPPPASRQGSTTNLQAAAGSSQNESNSSSTNSNSNATTTTTTTTTTTTNNNNNNNNNNNSSSNTNTPTTCTNCFTQTTPLWRRNPEGQPLCNACGLFLKLHGVVRPLSLKTDVIKKRNRGSGSSLPVGGGGTRKRGSTLSVSSSANPPPAQASTPPAASNRTGNANDSESSANGAASTGNGNTPTGYHGNAISSTNVAVGGKGVVPIAAAPAKNLPGPGAASVPRTTAISSSSLSSSKRQRRHSKSVTAAEQPSLNSMDIDSPESSTGSNEGAATSSAGLSKPSLGSSAGLASLQPSASSFSLGGFGSSGGSSGMQRPLAGPGSQRPAPQEWEWLTMSL

SEQ ID NO: 34 - DNA, 3441 bp, Myceliophthora thermophila
atggcagcgaccacgacggattcctctgcggcgcctggtccgctggccccaatgcatcccaccatgacagagcacgactttcgctttccgcgaaggccagcagacgccgcgaccggggcgcggaaaaccgaccgactcgaatctgcctccggtaccaagccggtgccgccgacatcaacgtcgacggcggcctcgaggacggcgacgtcggggaccacgacggcagcggcgggcggcggcggattgcgtcccggcctgctcgatggccgcctgaacctaggcctggctcagggtggtggtgccgcccaccttgacctgctgcgcgcctccgccttcccgcccttccagcagagtgcccggcgagaggcccagagcctggaggagatgcagagggaagacccgctggccacccagatctggaagttttacgcaaagacaaaacagctcctgcccgcccaggagcgcatggaaaacctgacgtggcggatgatgcatctcaagttgcagaagacgaaggcagccaacacgacaaagtaggtttttttctttctttctttctttctttcccgtcttggtgtcccgcggattgtgtggtacttcgccggttggaggactgggagaaagaaagaaagaaagaaaagaaaagaaaagaaagaaagcttgggagagagccgggtagaaaaagaagaatgcttgctgacttctgtgggcaaacaaaggacaagtcgtgcatccagctcatcagcaaatacccccagtgggattgcgcaattgcgcaagtcgtcggaacatgccccgccacagttggacccgatgaacctggacgatttcattcacaacgataacgtcggaaccccggccggcctcgccttgaccccgaccccagaaacgatgcgccaggcggaggagaggtcggctcacaccacggcgacggccattcccatcaagaaccgcaaggagccgtcgtcgtcgtcgtcttcgtcgtcgtctcagcatcttgcaccccagtcggtgcccgttgccgcccaccagaggcctcaggacgaatttggctatctgcctcggcacccgcgcaaaactagcattgacgagaccggccaacgtgtaagttctctcgccttttgctgctgcgtacaggtcgtctttggagtcaaatccatcgcgccccttgaagccgtcttgtattttcgatgacacgagatgccttcctgctttttttgtgatggcggtctgatgatccattgcctctctctctagacccgcaaacgaccggccaatttctccccccacgtacccgccgtcaacagcggcctgtcgacaagtggactcgatgccgacgctgacctccaggagtactcactcgaccacttacaacatcccgccgccgccagccaaccgcccaatcagatgggagttcccttcccgctcgacaccttccgcttggggaacgacctgagcataagctctgccgggcccttccagcagaacttctccttctcgccggccacctcgccaatggcggcgaatgacccgttcgccagcctctataatgcctcctcgatgccttcgggctccctaggggccgacttctattcccctcccggctcggcctatcagtctgccgtttccacaccgcatcccctgggcgaggccgatggcttctactttggctccatggatgtgcggcagatgcgccaacatccgtataggaatggccctgccgccatggccaacccgctaggccaacagttcccctacggcgcgaatggaggaaacctgatgttcccggccgcatcgaccggccccgagacgtcctcggcttttacggcgccgagctctttcagccatgtcgacccgacccgggtgttccagcaagacctccctgctcgttcccccgccgtcgggctgggccaagagagcatgttctccttcggagcggattccgacgacgaggacggcggcgcctttgccgaccgcaatctgccaatctcgcaggacctctcgccccaaggaagcagtatggatgacggtgcgtttgactgttcgtcactgcagtgggatccatctctcccgggcaactttagcatgcaggcggctaggtatcctggcgggcctactcggaaacaggtgacaataggcggcaccacgaccgactacgtcgactcgaagggggattgggagggcggaaatctgaacaggtcgcagtcgcagcctttccgtacaacgaacggtcgcgtcgggaaggtaccgcggacagcatcgaccccgaggttgacaagcaagggcaatccgttcgacaggttggcacagtcgaccccgaactcgccgccagccgaggccaaccagaactcggccttctcctctgtctctacaagccgcccgtcctcgcctcctccagcttcgaggcagggttccacaaccaacctccaagccgccgcgggaagcagtcagaacgagagcaacagcagcagcacgaactccaactccaacgccaccaccaccaccaccaccaccactaccaccaccaccaacaacaacaacaacaacaacaacaacaacaacagttcctccaacactaacacccccacaacatgtaccaactgcttcacccagacaacgccgctctggcgccgcaatcccgaaggccaacccctctgcaatgcttgcggtcttttcctcaagctgcatggtgtggttcgcccgctgagcttgaagacggacgtcatcaagaagcggaaccgcggctccggttcgagtcttcctgtcggcggcggcggcacccggtcgaggaagaacggcggctcctctggggggataagcaagaggggctcgactttatccgtctcctccagcgccaaccctcccccagctcaggcgagcaccccgccggcagcctcgaaccggaccggtaacgccaacgacagcgaaagttccgcgaacggcgccgcgtcgaccgggaatggaaacacgccaaccggctaccacggcaatgccatctcctctaccaatgttgccgttggcggcaagggtgtcgttccgatcgctgcggccccggcgaagaaccttcccggcccgggcgccgcctccgtaccgcgcaccacagccatctcctcttcctcgttgtcttcgtcgaaacgccaacgccgccacagcaagagcgtcacagcggcagagcagccgtcgctgaacagcatggacatcgacagcccggagagctctacgggatccaacgagggcgcggcaacgtcgtcggcgggcctgtcgaagccgagcctgggttcgagcgcggggctggcttcgttgcagcccagtgctagtagctttagtttgggtggctttggcagtagcggcggcagtagcggcatgcagaggccattggccgggccaggaagccagcggcctgcgccacaggagtgggagtggttgaccatgagtttatag

SEQ ID NO: 35 - DNA, 3066 bp, Myceliophthora thermophila
atggcagcgaccacgacggattcctctgcggcgcctggtccgctggccccaatgcatcccaccatgacagagcacgactttcgctttccgcgaaggccagcagacgccgcgaccggggcgcggaaaaccgaccgactcgaatctgcctccggtaccaagccggtgccgccgacatcaacgtcgacggcggcctcgaggacggcgacgtcggggaccacgacggcagcggcgggcggcggcggattgcgtcccggcctgctcgatggccgcctgaacctaggcctggctcagggtggtggtgccgcccaccttgacctgctgcgcgcctccgccttcccgcccttccagcagagtgcccggcgagaggcccagagcctggaggagatgcagagggaagacccgctggccacccagatctggaagttttacgcaaagacaaaacagctcctgcccgcccaggagcgcatggaaaacctgacgtggcggatgatgcatctcaagttgcagaagacgaaggcagccaacacgacaaagacaagtcgtgcatccagctcatcagcaaatacccccagtgggattgcgcaattgcgcaagtcgtcggaacatgccccgccacagttggacccgatgaacctggacgatttcattcacaacgataacgtcggaaccccggccggcctcgccttgaccccgaccccagaaacgatgcgccaggcggaggagaggtcggctcacaccacggcgacggccattcccatcaagaaccgcaaggagccgtcgtcgtcgtcgtcttcgtcgtcgtctcagcatcttgcaccccagtcggtgcccgttgccgcccaccagaggcctcaggacgaatttggctatctgcctcggcacccgcgcaaaactagcattgacgagaccggccaacgtacccgcaaacgaccggccaatttctccccccacgtacccgccgtcaacagcggcctgtcgacaagtggactcgatgccgacgctgacctccaggagtactcactcgaccacttacaacatcccgccgccgccagccaaccgcccaatcagatgggagttcccttcccgctcgacaccttccgcttggggaacgacctgagcataagctctgccgggcccttccagcagaacttctccttctcgccggccacctcgccaatggcggcgaatgacccgttcgccagcctctataatgcctcctcgatgccttcgggctccctaggggccgacttctattcccctcccggctcggcctatcagtctgccgtttccacaccgcatcccctgggcgaggccgatggcttctactttggctccatggatgtgcggcagatgcgccaacatccgtataggaatggccctgccgccatggccaacccgctaggccaacagttcccctacggcgcgaatggaggaaacctgatgttcccggccgcatcgaccggccccgagacgtcctcggcttttacggcgccgagctctttcagccatgtcgacccgacccgggtgttccagcaagacctccctgctcgttcccccgccgtcgggctgggccaagagagcatgttctccttcggagcggattccgacgacgaggacggcggcgcctttgccgaccgcaatctgccaatctcgcaggacctctcgccccaaggaagcagtatggatgacggtgcgtttgactgttcgtcactgcagtgggatccatctctcccgggcaactttagcatgcaggcggctaggtatcctggcgggcctactcggaaacaggtgacaataggcggcaccacgaccgactacgtcgactcgaagggggattgggagggcggaaatctgaacaggtcgcagtcgcagcctttccgtacaacgaacggtcgcgtcgggaaggtaccgcggacagcatcgaccccgaggttgacaagcaagggcaatccgttcgacaggttggcacagtcgaccccgaactcgccgccagccgaggccaaccagaactcggccttctcctctgtctctacaagccgcccgtcctcgcctcctccagcttcgaggcagggttccacaaccaacctccaagccgccgcgggaagcagtcagaacgagagcaacagcagcagcacgaactccaactccaacgccaccaccaccaccaccaccaccactaccaccaccaccaacaacaacaacaacaacaacaacaacaacaacagttcctccaacactaacacccccacaacatgtaccaactgcttcacccagacaacgccgctctggcgccgcaatcccgaaggccaacccctctgcaatgcttgcggtcttttcctcaagctgcatggtgtggttcgcccgctgagcttgaagacggacgtcatcaagaagcggaaccgcggctccggttcgagtcttcctgtcggcggcggcggcacccgcaagaggggctcgactttatccgtctcctccagcgccaaccctcccccagctcaggcgagcaccccgccggcagcctcgaaccggaccggtaacgccaacgacagcgaaagttccgcgaacggcgccgcgtcgaccgggaatggaaacacgccaaccggctaccacggcaatgccatctcctctaccaatgttgccgttggcggcaagggtgtcgttccgatcgctgcggccccggcgaagaaccttcccggcccgggcgccgcctccgtaccgcgcaccacagccatctcctcttcctcgttgtcttcgtcgaaacgccaacgccgccacagcaagagcgtcacagcggcagagcagccgtcgctgaacagcatggacatcgacagcccggagagctctacgggatccaacgagggcgcggcaacgtcgtcggcgggcctgtcgaagccgagcctgggttcgagcgcggggctggcttcgttgcagcccagtgctagtagctttagtttgggtggctttggcagtagcggcggcagtagcggcatgcagaggccattggccgggccaggaagccagcggcctgcgccacaggagtgggagtggttgaccatgagtttatag

SEQ ID NO: 36 - protein, 876 aa, Aspergillus nidulans
MSGLTLGGGSGRVRPTQAAAAFPTSHDFADPDRSSVARNANRPSRRFAAPPQLSDDFSLESPTDSAQVHDNLLQDALFPEWKANSPRGVDSPDEMQKKDPLATQIWKLYSRTKAQLPNQERMENLTWRMMALSLRRQERERAQQQARASSQKSPVPGMSGIAQLRLSDRVSNTPTTTADTVSDAMNLDDFIIPFSPSDHPSPSTTKASEATTGAIPIKARRDQSASEATPVPASFPHPAQDQRRESEFGYVPRRVRKTSIDERQFFNLQIPSRKRPAESSPHVPPVSTSMLAHDPDFSHAVPEYTLDTSHGLSLQNQMNAQQLANAQNHTSPNMAFALDTFNLGDDPILPSAGPYQQQFTFSPSESPMTSGNPFANLYAQTPIASSLNSTDFFSPPPSGYQSTASTPQPAYDGEHSKYFDMPVDARSQRRVVPAYITQRSSNLSASLQPRYMYNQGGSSQDITQQNAHMGAQSSSMQSPGFSIPQHVDPTQVLNPNEFNGNHAAMFSFGADSDVEDDDGNQFSAGGLAMPAEFGDDSISDMNSNMAWETSYPNSFQSLPAFAAQHRKHVTIGSADMMDTPSEWNQGGSLGRTHESAASVSEVRNRDQDPRRQKIARTSSTPNTAQLLRQSMQNQSSHTSPNTPPESGLNSAAPSRPASPGGTKNGEQNGPTTCTNCFTQTTPLWRRNPEGQPLCNACGLFLKLHGVVRPLSLKTDVIKKRNRNSANSLAVGSSRVSKKSARKNSVQQVTPTAPTSSRAQSNTTSESPPAMPGSSGRGSGVVPIAAAPPKSSSAATTSPGTNNGCGAVQVAPKRQRRLEKASDVDMAESPSSTSSGGRSKVVPLAPAMPPAAVNPANHSIAAGQGASQEWEWLTMSL

SEQ ID NO: 37 - DNA, 2692 bp, Aspergillus nidulans
atgtctgggttgactctcgggggtggttctggccgtgtgcgaccgacacaagccgctgccgcttttcccacctcgcatgatttcgccgaccctgatcgctcgtccgtcgcccgcaatgccaaccggccctcccgccgtttcgctgcacctccgcagctgtccgacgacttctcgctcgagtcacccactgattccgctcaagttcacgacaacctcctccaggacgctttattccccgaatggaaggccaattctcctcgaggggtcgacagccctgatgagatgcagaagaaggatccgttagcgactcaaatatggaagctctattctagaacgaaagctcagttgcccaaccaagagcgcatggaaaatttgacctggcggatgatggctctgagtctgaggcgtcaggaaagagagcgcgcccagcagcaagcccggtacgtcatcattcccgatcgggggtatttcttttctctttctcttgctgatcttctatagtgcctccagccaaaagagtcctgttcctggcatgagtggcatcgctcagctccgtctgtctgatcgtgtttccaataccccgaccacaactgcagatactgtctcagatgctatgaaccttgacgacttcatcatccctttctctccgtctgaccacccatctccctccacaacgaaagcctccgaagccactactggtgctatacctatcaaagcccgcagggatcagtccgcgtcggaagccaccccagtacctgcatctttccctcacccggcccaggatcagcggagggagagtgagttcggatacgtaccgcgtagagtgcgcaagacgagcatcgatgagcgtcaattcttcaacctgcagattccgtcccgcaagcggccggccgagtcctcgcctcacgtaccgcctgtgtccaccagcatgctagctcatgacccggatttctcacatgctgtaccggagtataccttggatacttcgcatggactgtctctacagaatcagatgaacgctcagcagctcgccaatgctcagaaccacacctcccccaatatggcgttcgctctggatactttcaacctgggcgacgatcctatccttccatctgctggtccttaccagcagcaattcactttctcgcccagcgagtcaccgatgaccagcggtaacccgtttgccaacctctacgctcagactccgattgcctcttcactcaactcaaccgacttcttctctcctccgccgtccggctatcagtcgactgcttcgactcctcagcctgcgtatgatggcgaacactccaagtattttgatatgcccgtcgacgcgcggtcgcaacgccgggttgttcccgcttacattactcagcgatcatcgaatttgtctgcctcgctgcaacctcgatatatgtacaaccagggtggttcgtcccaggatattacacagcagaatgcccatatgggggcccagtcttcatccatgcaatcccctggcttttctatcccgcagcatgttgatccaacgcaagtgttgaatccgaacgagtttaacggtaaccacgccgcaatgttcagtttcggcgccgactcggatgttgaagatgacgatgggaaccaattttcggccggaggactggcaatgccagccgagttcggcgacgacagtatcagtgacatgaatagcaacatggcgtgggagacatcatatcccaactctttccagtcgctgcctgcttttgctgcacagcaccgaaagcatgttaccatcgggtctgcggacatgatggataccccgagcgagtggaaccagggcggaagcctaggtcgaactcacgagtcggctgcgtcagtcagcgaggtgcgcaatcgggatcaagaccctcgacggcagaagatcgctcgcacctcgtccactccaaacacagcccaactactacgccagagcatgcagaaccaatcgtcccacacgtctcccaacactccgcccgagtcgggtctaaacagcgctgctccatcgcggcccgccagtccaggcgggaccaagaacggagagcagaatggtcccaccacctgcacgaactgctttacgcagaccacaccgctgtggcggcgtaaccctgaaggtcagccgctgtgcaacgcctgcggtttgtttttgaagttgcacggcgtggtgcgcccactgtcgctcaagaccgatgtgatcaagaagcgtaaccgcaacagcgccaatagccttgccgttggctcttcacgggtatccaaaaagtcagcccgcaagaactcggttcagcaggtaactccgactgcgcctacctcgagccgggcccaaagcaatacgacatccgagtctcccccagcaatgccgggtagctccggccgaggctctggagtcgttcctattgcggctgccccgcctaaatctagctcagcggcgactacgtctccgggcacaaacaacggttgtggtgcggtacaggtggctcctaaacgtcagcgtaggctggaaaaggcctctgatgttgatatggcggaaagcccgtccagtacctcgtccggtggacgatccaaggttgttccattggcgccggctatgcctccagcggccgtcaacccagccaaccacagtatcgctgctggtcagggcgcaagccaggaatgggagtggttgacgatgagtttgtag

SEQ ID NO: 38 - DNA, 2631 bp, Aspergillus nidulans
atgtctgggttgactctcgggggtggttctggccgtgtgcgaccgacacaagccgctgccgcttttcccacctcgcatgatttcgccgaccctgatcgctcgtccgtcgcccgcaatgccaaccggccctcccgccgtttcgctgcacctccgcagctgtccgacgacttctcgctcgagtcacccactgattccgctcaagttcacgacaacctcctccaggacgctttattccccgaatggaaggccaattctcctcgaggggtcgacagccctgatgagatgcagaagaaggatccgttagcgactcaaatatggaagctctattctagaacgaaagctcagttgcccaaccaagagcgcatggaaaatttgacctggcggatgatggctctgagtctgaggcgtcaggaaagagagcgcgcccagcagcaagcccgtgcctccagccaaaagagtcctgttcctggcatgagtggcatcgctcagctccgtctgtctgatcgtgtttccaataccccgaccacaactgcagatactgtctcagatgctatgaaccttgacgacttcatcatccctttctctccgtctgaccacccatctccctccacaacgaaagcctccgaagccactactggtgctatacctatcaaagcccgcagggatcagtccgcgtcggaagccaccccagtacctgcatctttccctcacccggcccaggatcagcggagggagagtgagttcggatacgtaccgcgtagagtgcgcaagacgagcatcgatgagcgtcaattcttcaacctgcagattccgtcccgcaagcggccggccgagtcctcgcctcacgtaccgcctgtgtccaccagcatgctagctcatgacccggatttctcacatgctgtaccggagtataccttggatacttcgcatggactgtctctacagaatcagatgaacgctcagcagctcgccaatgctcagaaccacacctcccccaatatggcgttcgctctggatactttcaacctgggcgacgatcctatccttccatctgctggtccttaccagcagcaattcactttctcgcccagcgagtcaccgatgaccagcggtaacccgtttgccaacctctacgctcagactccgattgcctcttcactcaactcaaccgacttcttctctcctccgccgtccggctatcagtcgactgcttcgactcctcagcctgcgtatgatggcgaacactccaagtattttgatatgcccgtcgacgcgcggtcgcaacgccgggttgttcccgcttacattactcagcgatcatcgaatttgtctgcctcgctgcaacctcgatatatgtacaaccagggtggttcgtcccaggatattacacagcagaatgcccatatgggggcccagtcttcatccatgcaatcccctggcttttctatcccgcagcatgttgatccaacgcaagtgttgaatccgaacgagtttaacggtaaccacgccgcaatgttcagtttcggcgccgactcggatgttgaagatgacgatgggaaccaattttcggccggaggactggcaatgccagccgagttcggcgacgacagtatcagtgacatgaatagcaacatggcgtgggagacatcatatcccaactctttccagtcgctgcctgcttttgctgcacagcaccgaaagcatgttaccatcgggtctgcggacatgatggataccccgagcgagtggaaccagggcggaagcctaggtcgaactcacgagtcggctgcgtcagtcagcgaggtgcgcaatcgggatcaagaccctcgacggcagaagatcgctcgcacctcgtccactccaaacacagcccaactactacgccagagcatgcagaaccaatcgtcccacacgtctcccaacactccgcccgagtcgggtctaaacagcgctgctccatcgcggcccgccagtccaggcgggaccaagaacggagagcagaatggtcccaccacctgcacgaactgctttacgcagaccacaccgctgtggcggcgtaaccctgaaggtcagccgctgtgcaacgcctgcggtttgtttttgaagttgcacggcgtggtgcgcccactgtcgctcaagaccgatgtgatcaagaagcgtaaccgcaacagcgccaatagccttgccgttggctcttcacgggtatccaaaaagtcagcccgcaagaactcggttcagcaggtaactccgactgcgcctacctcgagccgggcccaaagcaatacgacatccgagtctcccccagcaatgccgggtagctccggccgaggctctggagtcgttcctattgcggctgccccgcctaaatctagctcagcggcgactacgtctccgggcacaaacaacggttgtggtgcggtacaggtggctcctaaacgtcagcgtaggctggaaaaggcctctgatgttgatatggcggaaagcccgtccagtacctcgtccggtggacgatccaaggttgttccattggcgccggctatgcctccagcggccgtcaacccagccaaccacagtatcgctgctggtcagggcgcaagccaggaatgggagtggttgacgatgagtttgtag

SEQ ID NO: 39 - DNA, 21 bp, Artificial Sequence
  Primer
gggcaatcacccgctttttcc

SEQ ID NO: 40 - DNA, 20 bp, Artificial Sequence
  Primer
cccgaaccaaaagctctgat

SEQ ID NO: 41 - DNA, 24 bp, Artificial Sequence
  Primer
ccacgggaatctccaccgttctca

SEQ ID NO: 42 - DNA, 25 bp, Artificial Sequence
  Primer
tacagtagacagcgagcaaccacag

SEQ ID NO: 43 - protein, 119 aa, Artificial Sequence
  VHH1
DVQLVESGGGLVQAGGSLRLSCAASRSIFSINAMDWYRQAPGKQREWVAGITRGGTTKYADSVKGRFTISRDNAKKKVYLQMNSLKPEDTAVYYCNVLRGEQPWTRDYWGQGTQVTVSS

SEQ ID NO: 44 - protein, 119 aa, Artificial Sequence
  VHH1 Q
QVQLVESGGGLVQAGGSLRLSCAASRSIFSINAMDWYRQAPGKQREWVAGITRGGTTKYADSVKGRFTISRDNAKKKVYLQMNSLKPEDTAVYYCNVLRGEQPWTRDYWGQGTQVTVSS

SEQ ID NO: 45 - protein, 10 aa, Artificial Sequence
  VHH1 CDR1
RSIFSINAMD

SEQ ID NO: 46 - protein, 9 aa, Artificial Sequence
  VHH1 CDR2
GITRGGTTK

SEQ ID NO: 47 - protein, 11 aa, Artificial Sequence
  VHH1 CDR3
LRGEQPWTRDY

SEQ ID NO: 48 - protein, 117 aa, Artificial Sequence
  VHH2
QVQLQESGGGLVQAGGSLRLSCAASGTIFRPTAMGWYRQAPGKERELVATITTGGSTKYADSVKGRFTISRGNAKNTVYLQMSSLKPEDTAVYYCNAQWGVRTRDYWGQGTQVTVSS

SEQ ID NO: 49 - protein, 10 aa, Artificial Sequence
  VHH2 CDR1
GTIFRPTAMG

SEQ ID NO: 50 - protein, 9 aa, Artificial Sequence
  VHH2 CDR2
TITTGGSTK

SEQ ID NO: 51 - protein, 9 aa, Artificial Sequence
  VHH2 CDR3
QWGVRTRDY

SEQ ID NO: 52 - protein, 126 aa, Artificial Sequence
  VHH3
QVQLQESGGGLVQAGDSLRLSCAASISDRAFSRHVMGWFRQPPGKEREFVAAIGWTGRRTYYADSVKGRFTISRDNAMNTVYLQMNSLKPEDTAVYYCAASHFYSVSFEINDYDYWGQGTQVTVSS

SEQ ID NO: 53 - protein, 10 aa, Artificial Sequence
  VHH3 CDR1
ISDRAFSRHV

SEQ ID NO: 54 - protein, 10 aa, Artificial Sequence
  VHH3 CDR2
AIGWTGRRTY

SEQ ID NO: 55 - protein, 15 aa, Artificial Sequence
  VHH3 CDR3
SHFYSVSFEINDYDY

SEQ ID NO: 56 - protein, 117 aa, Artificial Sequence
  VHH2 D
DVQLQESGGGLVQAGGSLRLSCAASGTIFRPTAMGWYRQAPGKERELVATITTGGSTKYADSVKGRFTISRGNAKNTVYLQMSSLKPEDTAVYYCNAQWGVRTRDYWGQGTQVTVSS

SEQ ID NO: 57 - protein, 126 aa, Artificial Sequence
  VHH3 D
DVQLQESGGGLVQAGDSLRLSCAASISDRAFSRHVMGWFRQPPGKEREFVAAIGWTGRRTYYADSVKGRFTISRDNAMNTVYLQMNSLKPEDTAVYYCAASHFYSVSFEINDYDYWGQGTQVTVSS

SEQ ID NO: 58 - protein, 944 aa, Trichderma Reesei
MAAVGPLAMHPTVTEHDYRFPRRPDPHRSTDRSFARMDSRDDHVALANGNASAAVTSGQAASSRKNSVLDSALFPSLENAASGSSSSLEQMQSDDPLAAQIWKFFNRTKQQLPNQQRMENLTWRMMALNLRKRQQQQQQQKTHNSQPQNAPSGIAQQLRKTSDSHAEGVDAMNIDDFIFAEEGGASVKDNSPQPPASSLSGPQTAATKTSKSSRPLASAIPIKGRKGSSANQFVAQSVPVAPPHQRGQFDEFHYVPRHDRKTSIDDRRTRKRPANFSPHVSAINPSASGLSLDLEADPDLNGYSLDNTNAMSLQQQMANNQGSAFNLDFMDSDGVLGSAPFHPNFSFSPSTSPMMHNGPFPSVYNNAALQSSLNNPDFYSPTGSGYPSATSTPHPMNEGDGVYFGSQEMQHHPHHRAQGFRQSSGNLNHHLSHQFMYGDSNGGNGSGGGNGAAFPSTGASSEAVASYSAAQNSFGHIDPSQVFQTESQVASPVLPMQQGNMLGFGGESDEDDGTAFLDRGGVGLHGDFTSAVDEAGTFSWDASLPGQFSTQAARFPGGPTRKHVQIGTITTDYVDTSNDWDGHNLSRSHSQTLGMGNDKRQNKLPRNSSTPSHMAAKHSGFEQIAQSLPSSPGPHASGTASGFSSVAPSRPASPPPSSKHGSSTNLHAASGNPGENSAPTTCTNCFTQTTPLWRRNPEGQPLCNACGLFLKLHGVVRPLSLKTDVIKKRNRGSGANLPVGGSRKKNAGNGSNAASRKNSSLSMAAAAAATAAVATANHQAATPPSTTRPSSNKEGDSPASSGHHTAGSTPGSQYGGATIVGGKGVVPIAAAPPKTTPGPGASSSMSRPTTASSKRQRRHSKSEGTSSSAMDLDGPGDSIGRQLGATPSMPSISSTMLSNGSFTMGQRSMMGSSMVSMGGSQGNLVNSAGGSSGPQEWEWLTMSL

SEQ ID NO: 59 - protein, 1021 aa, Myceliophthora heterothallica
MAATTTDSSAAPGPLAPMHPTTTEHDFRFPRRPPADAATGARKTDRLESASGTKPAPPTSTSTATPRTATAGTTTATAGADGLRPGLLDGRLNLGLAQSGGAAHLDLLRVSAFPPFQQSARREAQSLEEMQREDPLATQIWKFYAKTKQLLPAQERMENLTWRMMHLKLQKTKAANTTKTSRASSSSANTPSGIAQLRKSSEHAPPQLDPMNLDDFIHNDNVGTLASLALTPTPETMRQAEERSAHTTATAIPIKNRKEPSSSSSSSSSSQHLAPQSVPVAAHQRPRDEFGYLPRHPRKTSIDETGQRTRKRPANFSPHVPAVNSGLSTSGLDADADLQEYSLDHLQHPAAASQPPNQMGVPFPLDTFRLGNDLSISSAGPFQQNFSFSPATSPMAANDPFASLYNSSSMPSGSLGADFYSPPGSAYQSAVSTPHPLGEADGFYFGSMDVRQMRQHPYRNGPAAMANPLGQQFPYGANGSLMFPAASTGPETSSAFTAPSSFSHVDPTRVFQQELPARSPAVGLGQESMFSFGADSDDEDGGAFADRNLPISQDLSPQGSSMDDGAFDSSSLQWDPSLPGNFSMQAARYPAGPTRKQVTIGGTTTDYVDSKGDWEGGSLNRSQSQPFRTTNGRVGKVPRTASTPRLTSKGNPFDRLAQSTPNSPPAEANQNSAFSSVSTSRPSSPPPASRQGSTTNLQAAAGSSQNESSSSGGGGGSSGSTNPNSNASNNNNNNNNNNNNNSSSNTNTPTTCTNCFTQTTPLWRRNPEGQPLCNACGLFLKLHGVVRPLSLKTDVIKKRNRGSGSSLPVGGGGTRKRGSTLSVSSSANPPPAQASTPPAASNRTGSANDSESSANGAASTGNGNTPTGYHGNATSSTNVAVGGKGVVPIAAAPAKNLPGPGAASIPRTTAISSSSLSSSKRQRRHSKSVTAAEQPSLNSMDIDSPESSTGSNEGAATSSAGLSKPSLGSSAGLASLQPSASSFSLGGFGSSGGSNGMQRPLAGPGSQRPAPQEWEWLTMSL

(19) United States
(12) Patent Application Publication
(10) Pub. No.: US 2024/0279595 A1
(43) Pub. Date: Aug. 22, 2024

(54) EXPRESSION HOST

(71) Applicant: BIOTALYS NV, Sint-Denijs-Westrem (BE)

(72) Inventor: Lucia Nancy Coconi Linares, Sint-Denijs-Westrem (BE)

(21) Appl. No.: 18/007,317

(22) PCT Filed: Aug. 2, 2021
(86) PCT No.: PCT/EP2021/071595

§ 371 Date: Jan. 30, 2023

(30) Foreign Application Priority Data
Jul. 31, 2020 (EP) 20188921.9
Jul. 31, 2020 (EP) 20188933.4
Dec. 30, 2020 (EP) 20217907.3

(51) Int. Cl. C12N 1/14; C07K 14/38; C07K 16/00; C12N 15/80; C12P 21/02

(52) U.S. Cl. CPC C12N 1/14; C07K 14/38; C07K 16/00; C12N 15/80; C12P 21/02; C07K 2317/14; C07K 2317/569; C12N 2510/00

(57) ABSTRACT

The present invention relates to modified microbial cells, the modification modulates protease activity if compared with a parent microbial cell which has not been modified and measured under the same or substantially the same conditions. The present invention further relates to a method for the manufacturing of polypeptides. The present invention further provides an improved method of producing polypeptides wherein increased yields are obtained. The present invention also relates to a method of producing the microbial cells of the invention. The present invention provides nucleic acids, genetic constructs, host cells and kits for use in the method of the invention as well as polypeptides obtained by the method of the invention.

### CROSS-REFERENCE TO RELATED APPLICATIONS

This application is a national phase application filed under 35 U.S.C. § 371 claiming benefit to International Patent Application No. PCT/EP2021/071595, filed Aug. 2, 2021, which claims the benefit of priority from European Patent Application 20188921.9, filed Jul. 31, 2020, European Patent Application 20188933.4, filed Jul. 31, 2020, and European Patent Application 20217907.3, filed Dec. 30, 2020, the contents of each of which are hereby incorporated by reference in their entirety.

Reference to a “Sequence Listing”, a table, or a computer program listing appendix submitted as an ASCII text file

The present application hereby incorporates by reference the entire contents of the text file named “206189-0047-00US_SequenceListing2” in ASCII format, which was created on Aug. 17, 2023, and is 97,754 bytes in size.

### FIELD OF THE INVENTION

The present invention relates to modified microbial cells, such as modified host cells. More specifically, the present invention relates to the modified microbial cells wherein the modification modulates protease activity if compared with a parent microbial cell which has not been modified and measured under the same or substantially the same conditions. The present invention further relates to a method for the manufacturing of polypeptides. The present invention further provides an improved method of producing polypeptides wherein increased yields are obtained. The present invention also relates to a method of producing the microbial cells of the invention. The present invention provides nucleic acids, genetic constructs, host cells and kits for use in the method of the invention as well as polypeptides obtained by the method of the invention.

Other aspects, embodiments, advantages and applications of the invention will become clear from the further description herein.

### BACKGROUND

The efficient and cost-effective production of recombinant proteins is very important in the field of pharmacology but even more so in the field of agriculture where greater amounts of active protein may be required at low cost price. This puts high demands on the production process and development of biological products.

Different species of filamentous fungi have historically been used in fermentations and were selected by centuries of use. In more recent times, filamentous fungi are being used for their properties to produce extracellular plant biomass-degrading enzymes. This interesting aspect was mainly exploited with the production of biofuels as a goal. The key producers of extracellular (hemi)-cellulases are Aspergillus, Trichoderma, Penicillium and Neurospora species and over the past decades these strains have been improved using random mutagenesis, selection and genetic engineering with some species and strains now reported to produce up to 100 g/l of extra-cellular (hemi)cellulases (Cherry J R, Fidantsef A L, Opin. Biotechnol. 14(4), 438-443). Such protein production levels have spurred researchers to try and utilize filamentous fungi for the production of recombinant proteins by using strong endogenous promoters, signal peptides, and carrier (hemi)cellulolytic genes fused to the target genes. Very often however, these attempts did not produce the desired or hoped for expression levels of recombinant proteins. For example, during the production of a biological product, such as conventional monoclonal antibodies, unsatisfactory yields were reported ranging from 0.15 g/l in T. reesei to 0.9 g/l in A. niger. Such low amounts of biological product are insufficient for profitable production of proteins in industrial biotechnology, pharmacological and agricultural applications (Nyyssönen et al, 1993, Biotechnology. 11; Ward et al 2004, Appl. Environ. Microbiol. 70).

Many efforts have been undertaken to increase expression levels from filamentous fungi, such as searching for new promoters, deleting regulators such as catabolite repression modulators, introduction of chaperones, and so forth (Nevalainen, 2004, Handbook of fungal biotechnology). But despite all these previous and ongoing efforts, no substantial progress has yet been reported in the yields of recombinant protein production in fungal hosts (Nevalainen et al 2014, Front. Microbiol. 5:75).

A key reason might be the rapid degradation of secreted (heterologous) recombinant proteins by the presence of extracellular proteases. Indeed, filamentous fungi are well-known for secreting a wide variety and large amounts of proteases into the environment. Proteins that are unstable or sensitive to protease degradation will therefore quickly be degraded. This results in very low protein yields or even the total absence of protein recovery after fermentation due to large quantities of these proteases in the fermentation broth. Additionally, the presence of proteases in a protein formulation, be it even at low quantities, may greatly impact the shelf life of protein products. Therefore, many attempts have been undertaken to reduce the protease activity and hence stability of recombinant proteins in the culture media. A tested approach is the deletion of each individual protease as identified by homology searches and certain discernible patterns shared by commonly known proteases. WO2013102674, WO2015004241 and WO2007045248 describe Trichoderma mutants with a plurality of individually modified protease genes in an attempt to reduce degradation of a recombinantly produced biological products. In addition to the laborious and time consuming procedure to modify each protease sequentially, it also comes with the drawback that it is limited to those proteases that have been identified by experimental or bio-informatic analysis. It is likely that many proteases remain unidentified. And even if all proteases are identified, the deletion of all of them would ideally be necessary.

Alternatively, protease regulators are modified. WO2017025586 reports the modification of T. reesei genes that share characteristics of regulators of transcription. This identification was also based on the proximity of those regulators to protease genes or clusters of proteases. The inactivation of 3 putative regulators and the deletion of 8 individual proteases led to decreases in protease production and increased yields in interferon production of 3.7-fold compared to a parent strain. WO2017025586 does not test production of other biologicals such as traditional monoclonal antibodies.

WO2016132021 describes the inactivation of a newly discovered regulator, pea1, and reports reduced protease activity to a level of 25-50% compared to that of wild type levels in Trichoderma reesei and a 40-fold reduction in protease levels when pea1 was inactivated in Fusarium oxysporum. No increases in protein production yields were reported. However, Qian et al. (2019) report the deletion of a regulator, Are1, in the Trichoderma reesei strain QM9414. Whilst reporting decreased expression of two proteases Apw1 (Uniport ID: GOR8TO) and Apw2 (Uniprot ID: GOR9K1), no increased production or improved stability of a compound of interest was reported. Unfortunately, the Are1 deletion in QM9414 drastically reduced expression of the major cellulase proteins Cbh1 and Cbh2 in the presence of ammonium sulphate and peptone, reducing the industrial applicability of this modified Trichoderma strain since cbh1 and cbh2 promoters are very often used to drive expression of a heterologous protein to high levels.

Although WO2013102674, WO2015004241 and WO2007045248 report the specific inactivation of proteases and WO2017025586, WO2016132021 and Qian et al. (2019) report the inactivation of specific genetic regulators, with both approaches leading to a decreased protease content, reported results in the reduction of recombinant protein degradation remain highly specific for the chosen protease, highly variable or even absent. Significant increases in protein yields from large scale fermentations remain to be reported.

There remains a need in the art for still further improved host cells, for example filamentous fungal cells, such as Trichoderma fungus cells, that can stably produce heterologous proteins, such as immunoglobulins, preferably at high levels of expression.

### SUMMARY OF THE INVENTION

The present invention provides modified microbial host cells, which may be suitable for the production of compounds of interest, in particular recombinant proteins.

In a first aspect of the invention there is provided a modified microbial cell, such as a microbial host cell, which is characterized by:
a. having been modified and where this modification affects the production, stability and/or function of at least one polypeptide; and
b. having a modulation in protease activity if compared with a parent microbial host cell which has not been modified and is measured under the same or substantially the same conditions.

In a second aspect of the invention, there is provided a method of producing a modified microbial host cell comprising the steps of: providing a parent microbial host cell; and modifying the parent microbial host cell, to yield a modified microbial host cell having a modulated production, stability and/or function of at least one polypeptide.

In a third aspect of the invention, there is provided a modified microbial host cell having a modulated activity of a polypeptide comprising a sequence according to a sequence selected from the group consisting of SEQ ID NOs: 1, 28, 33, 36, 58 and 59 or a polypeptide at least about 80%, at least about 85%, at least about 90%, at least about 95% or at least about 98% identical thereto, or an ortholog thereof, compared to a corresponding wild type modified microbial host cell that expresses said polypeptide.

In a fourth aspect of the invention, there is provided a method of producing a modified microbial host cell comprising the steps of: providing a parent microbial host cell; and modifying the parent microbial host cell, to yield a modified microbial host cell having a modulated activity of a polypeptide comprising a sequence according to a sequence selected from the group consisting of SEQ ID NOS: 1, 28, 33, 36, 58 and 59 or a polypeptide at least about 80%, at least about 85%, at least about 90%, at least about 95%, or at least about 98% identical thereto, or an ortholog thereof, compared to the activity of said polypeptide prior to the modification.

In a fifth aspect of the invention there is provided a method for the production of a compound of interest comprising: providing a modified microbial host cell of the invention, wherein the host cell is capable of expressing the compound of interest, culturing said modified microbial host cell under conditions conducive to the expression of the compound of interest, and optionally isolating the compound of interest from the culture medium.

In a further aspect of the invention, there is provided the use of a modified microbial host cell for the production of a compound of interest, wherein the microbial host cell is characterized by (a) having been modified and where this modification affects the production, stability and/or function of at least one polypeptide; (b) having a reduction or deficiency in protease activity if compared with a parent microbial host cell which has not been modified and is measured under the same or substantially the same conditions; and (c) comprising at least one polynucleotide coding for the compound of interest.

In a still further aspect of the invention, there is provided a kit of parts, wherein the kit comprises a microbial host cell and a vector encoding a compound of interest, the kit optionally further comprising a vector for the modification of at least one polypeptide expressed by the microbial host cell.

### BRIEF DESCRIPTION OF THE SEQUENCE LISTING

SEQ ID NO: 1 sets out the amino acid sequence of a target polypeptide of the invention (i.e. a polypeptide which is modified to affect its production, stability and/or function). This example is the sequence of Are1 from Trichoderma reesei.
SEQ ID NO: 2 sets out the genomic nucleotide sequence encoding a target polypeptide of the invention.
SEQ ID NO: 3 sets out a nucleotide sequence encoding a target polypeptide of the invention.
SEQ ID NOs 4-9: Primers used to obtain the DNA donors polypeptide deletions.
SEQ ID NOs 10-17: Primers used for sequencing analysis.
SEQ ID NOS 18-19: Primers used for the donor amplification of Are1 deletion in T. reesei.
SEQ ID NOS 20-27: Primers used for screening of donor cassette integration into the Are1 deletion region in T. reesei.
SEQ ID NO: 28 sets out the amino acid sequence of a further target polypeptide of the invention (i.e. a further polypeptide which is modified to affect its production, stability and/or function). This example is the sequence of AreA from Myceliophthora heterothallica.
SEQ ID NO: 29 sets out the genomic nucleotide sequence encoding the further target polypeptide of SEQ ID NO: 28.
SEQ ID NO: 30 sets out a nucleotide sequence encoding the further target polypeptide of SEQ ID NO: 28.
SEQ ID NO: 31 sets out the sequence of a GATA-type zinc finger domain present in target polypeptides of the invention.
SEQ ID NO: 32 is the DNA sequence bound by GATA-type zinc fingers.
SEQ ID NO 33 sets out the amino acid sequence of a further target polypeptide of the invention (i.e. a further polypeptide which is modified to affect its production, stability and/or function). This example is the amino acid sequence of AreA from Myceliophthora thermophila.
SEQ ID NO: 34 sets out the genomic nucleotide sequence encoding the further target polypeptide of SEQ ID NO: 36.
SEQ ID NO: 35 sets out the genomic nucleotide sequence encoding the further target polypeptide of SEQ ID NO: 36.
SEQ ID NO 36 sets out the amino acid sequence of of a further target polypeptide of the invention (i.e. a further polypeptide which is modified to affect its production, stability and/or function). This example is the amino acid sequence the amino acid sequence of AreA from Aspergillus nidulans
SEQ ID NO: 37 sets out the genomic nucleotide sequence encoding the further target polypeptide of SEQ ID NO: 39.
SEQ ID NO: 38 sets out the genomic nucleotide sequence encoding the further target polypeptide of SEQ ID NO: 39.
SEQ ID NOS 39-40: Primers used for the donor amplification for AreA deletion.
SEQ ID NOS 41-42: Primers used for screening of donor cassette integration into the AreA deletion region.
SEQ ID NOS: 43 to 47 are the sequence of VHH-1, where SEQ ID NO: 43 is the full length sequence of VHH-1, SEQ ID NO: 44 is the full length sequence of VHH-1 but in which the first residue is changed to a Q residue, SEQ ID NO: 45 is the CDR1 of VHH-1, SEQ ID NO: 45 is the CDR2 of VHH-1 and SEQ ID NO: 46 is the CDR3 of VHH-1.
SEQ ID NOs: 48 to 51 and 56 are the sequences of VHH-2, where SEQ ID NO: 48 is the full length sequence of VHH-1, SEQ ID NO: 56 is the full length sequence of VHH-2 but in which the first residue is changed to a D residue, SEQ ID NO: 49 is the CDR1 of VHH-2, SEQ ID NO: 50 is the CDR2 of VHH-2 and SEQ ID NO: 51 is the CDR3 of VHH-2.
SEQ ID NOS: 52 to 55 and 57 are the sequences of VHH-3, where SEQ ID NO: 52 is the full length sequence of VHH-1, SEQ ID NO: 57 is the full length sequence of VHH-3 but in which the first residue is changed to a D residue, SEQ ID NO: 53 is the CDR1 of VHH-3, SEQ ID NO: 54 is the CDR2 of VHH-3 and SEQ ID NO: 55 is the CDR3 of VHH-3.
SEQ ID NO: 58 is an alternative polypeptide sequence of Are1 from Trichoderma reesei.
SEQ ID NO: 59 is an alternative polypeptide sequence of AreA from Myceliophthora heterothallica.

The sequence identity (percentages) between SEQ ID NOs: 1, 28, 33, 36, 58 and 59 is shown below:

| | SEQ ID | SEQ ID | SEQ ID | SEQ ID | SEQ ID | SEQ ID |
|---|---|---|---|---|---|---|
| | NO: 1 | NO: 28 | NO: 33 | NO: 36 | NO: 58 | NO: 59 |
| T. Reesei Are1 (SEQ ID 1) | 100 | 43.5 | 43 | 36.7 | 99.4 | 43.4 |
| M. heterothallica AreA (SEQ ID 28) | 42.3 | 100 | 93.5 | 37.2 | 42.4 | 98.5 |
| M. thermophilla AreA (SEQ ID 33) | 43.2 | 94 | 100 | 36.7 | 43.2 | 94.4 |
| A. Nidulans AreA (SEQ ID 36) | 37.3 | 36.4 | 36.1 | 100 | 37.3 | 36.7 |
| T. Reesei Are1 (SEQ ID 58) | 99.4 | 43.6 | 43 | 36.7 | 100 | 43.5 |
| M. heterothallica AreA (SEQ ID 59) | 48.1 | 98.5 | 94.9 | 37.1 | 48.1 | 100 |

### BRIEF DESCRIPTION OF THE FIGURES

FIG. 1: Schematic representation of the fusion deletion cassette Are1-hyg donor pJET

FIG. 2: SDS-PAGE analysis of extracellular proteins of the modified Trichoderma reesei (TF1, TF2, TF3, TF4, TF5, and TF6), control (Mut), and wild type (WT) at different time points post-lactose induction. CTL shows the pure VHH-1 as a reference. A and B indicate the different biological replicates analyzed.

FIG. 3: SDS-PAGE analysis of extracellular proteins of the modified Trichoderma reesei (TF1, TF2, TF3, TF4, TF5, TF6) and a control (Mut) and wild type (WT) after 6 days post-lactose induction in either Vogel's medium with ammonium or peptone as a nitrogen source. CTL shows the pure VHH-1 as a reference.

FIG. 4: pNP-cellobiohydrolase assays of the modified Trichoderma reesei (TF1 to TF6), control strain (MUT), wild type (WT) and a blank (CTL) after 11 days of fermentation.

FIG. 5: Effect of the composition of the culture media using lactose (LAC) or sophorose (SOP) as inducers on the production of extracellular proteases of modified Trichoderma reesei cells (Aare) compared to the parental microbial host cells (Trichoderma reesei RL-P37; abbreviated as P37).

FIG. 6: Comparison of protein abundance of CBHI (A) and CBHII (B) detected after 6 days growth of modified Trichoderma reesei cells and parental microbial host cells on minimal media (Trire) and Vogels medium (Vog) supplemented with lactose (LAC) or sophorose (SOP). The bars show the label-free quantification (LFQ) intensities associated with the cellulase abundance.

FIG. 7: Schematic representation of the fusion deletion cassette AreA-neoR donor pIDT.

FIG. 8: SDS-PAGE analysis of extracellular proteins ofthe modified M. heterothallica (AareA transformants TF1-TF4), wild type (WT), and negative control (CTL-) after 3 days post-lactose-avicel induction. CTL shows the pure VHH-1 as a reference.

FIG. 9: Extracellular protease production by M. heterothallica AareA transformants (TF1-TF4) and parental M. heterothallica cells (WT).

FIG. 10: Production of VHH-1 using CBHI catalytic domain as secretion carrier from the cbhl promoter (Panel A left hand side). Panel A right hand side shows a corresponding western blot. Panel C shows western blot for VHH-1 production lacking the CBHI catalytic domain. Panel C shows production of refVHH.

FIG. 11: Rapid degradation of spiked VHH-1 in the culture broth of T. reesei and M. heterothallica cells modified in the phoG and xprG transcription factors.

### DETAILED DESCRIPTION OF THE INVENTION

Reference to any prior art in this specification is not, and should not be taken as, an acknowledgment or any form of suggestion that this prior art forms part of the common general knowledge in any country. All documents cited in the present specification are hereby incorporated by reference in their entirety. Unless otherwise defined, all terms used in disclosing the invention, including technical and scientific terms, have the meaning as commonly understood by one of ordinary skill in the art to which this invention belongs. The present invention will be described with respect to particular embodiments but the invention is not limited thereto but only by the claims. Any reference signs in the claims shall not be construed as limiting the scope.

Where the term “comprising” is used in the present description and claims, it does not exclude other elements or steps.

Where an indefinite or definite article is used when referring to a singular noun e.g. “a” or “an”, “the”, this includes a plural of that noun unless something else is specifically stated.

The term “about” as used herein when referring to a measurable value such as a parameter, an amount, a temporal duration, and the like, is meant to encompass variations of +/−10% or less, preferably +/−5% or less, more preferably +/−1% or less, and still more preferably +/−0.1% or less of and from the specified value, insofar such variations are appropriate to perform in the disclosed invention. It is to be understood that the value to which the modifier ‘about’ refers is itself also specifically, and preferably, disclosed.

The following terms or definitions are provided solely to aid in the understanding of the invention. Unless specifically defined herein, all terms used herein have the same meaning as they would to one skilled in the art of the present invention. Practitioners are particularly directed to Sambrook et al., Molecular Cloning: A Laboratory Manual, 2nd ed., Cold Spring Harbor Press, Plainsview, New York (1989); and Ausubel et al., Current Protocols in Molecular Biology (Supplement 47), John Wiley & Sons, New York (1999), for definitions and terms of the art. The definitions provided herein should not be construed to have a scope less than understood by a person of ordinary skill in the art.

Unless indicated otherwise, all methods, steps, techniques and manipulations that are not specifically described in detail can be performed and have been performed in a manner known per se, as will be clear to the skilled person. Reference is for example again made to the standard handbooks, to the general background art referred to above and to the further references cited therein.

The following invention relates to a microbial cell, such as a microbial host cell, which has been modified, and where this modification affects the production, stability and/or function of a polypeptide, for example a polypeptide according to a sequence selected from the group consisting of SEQ ID NOs: 1, 28, 33, 36, 58 and 59 or a polypeptide at least 80% identical thereto, or an ortholog thereof, and where this microbial host cell has a modulation in protease activity if compared with a parent microbial host cell which has not been modified and when measured under the same or substantially the same conditions. In preferred embodiments, the modulation in protease activity is a reduction or deficiency in protease activity. A reduction or deficiency in protease activity may be particularly suited for embodiments relating to the provision of a compound of interest, in particular a proteinaceous compound of interest.

For example, it has been surprisingly found that when the modified microbial host cell according to the invention and which is further capable of expressing a compound of interest is used in a method to produce a compound of interest, for example a polypeptide, an improved yield of said compound is obtained if compared to a method in which a parent host cell is used and measured under the same or substantially the same conditions.

In addition, it has been found that when the modified microbial host cell according to the invention which has been modified to adversely affect the production, stability and/or function of the polypeptide and which is capable of expressing a compound of interest is used in a method to produce a compound of interest, the fermentation broth or cell culture medium comprising the microbial host cell and/or the intracellular environment of the microbial host cell may demonstrate a reduction in protease activity compared to a method in which a parent host cell is used and measured under the same or substantially the same conditions.

Surprisingly, the reduction in protease activity in the fermentation broth (or cell culture medium) or intracellular environment of the modified microbial host cell according to this invention and capable of expressing a compound of interest, increases in the yield of the compound of interest, such as a polypeptide produced by the host cell. Additionally, the reduced production and activity of proteases from the host cell according to this invention can lead to increased stability of the compound of interest leading to more functional and intact compounds being produced. Furthermore, the reduced production and activity of proteases from the host cell according to this invention may lead to an increased shelf-life and storage stability of the compound of interest produced by the microbial host cell according to this invention.

The microbial host cell according to this invention and the production of a compound of interest can be useful for the industrial production of compounds of interest such as polypeptides. The polypeptides may be useful in the preparation of agrochemical or pharmaceutical compositions.

Microbial Host Cells and Methods for Making them

The present invention provides modified microbial cells, specifically microbial host cells. This modification affects the production, stability and/or function of one or more polypeptides.

Within the context of the present invention “measured under the same conditions” or “measured under substantially the same conditions” means that the microbial host cell which has been modified and the parent microbial host cell are cultured under the same conditions and a certain aspect related to the microbial host cell is measured in the microbial host cell which has been modified, and in the parent host cell, respectively, using the same conditions, preferably by using the same assay and/or methodology, more preferably within the same experiment. The same conditions refers to the culture conditions used to culture the parent and modified microbial host cell. The same conditions may also refer to the use of the same assay to determine protease activity in a cultured parent microbial host cell and a cultured modified microbial host cell.

For example, in some embodiments, the method for measuring protease activity comprises providing a microbial cell whose protease activity is to be measured, culturing the microbial host cell in a cell culture medium, and measuring the level of protease activity in the culture broth, for example either by obtaining a sample of the culture broth and determining its protease activity by measuring the ability of the broth sample to degrade a test protein, or spiking the culture broth with a test protein (i.e. adding a quantity of test protein to the cell culture medium) and measuring the extent of the degradation of the test protein in the culture broth over time, or by identifying and/or quantifying the proteases present in the broth sample by mass spectrometry techniques for example by by liquid chromatography-tandem mass spectrometry (LC-MS/MS).

In some embodiments, the method for measuring protease activity comprises providing a microbial cell whose protease activity is to be measured, culturing the microbial cell in a liquid cell culture medium at 30° C. for 48 hours, followed by adding a test protein to the liquid cell culture medium (for example 500 μL of monoclonal antibody solution having a concentration of 30 mg/mL), obtaining one or more samples of the liquid cell culture medium at periodic intervals and measuring the level of test protein in each sample to determine the protease activity of the microbial host cell. The method may further comprise carrying out the same method on a test microbial host cell that has not been modified (i.e. a parent microbial host cell) and comparing the rate and/or extent of degradation of the test protein with the modified microbial host cell to quantify the change in protease activity caused by the modification to the microbial host cell.

Other methods of determining a change in protease activity as a result of one or more modifications to the microbial host cell will be apparent to the skilled person. For example, when the microbial host cell comprises a nucleotide sequence coding for a compound of interest (i.e. a heterologous nucleotide sequence coding for a compound of interest), the method may comprise culturing the microbial host cell in a cell culture medium under conditions to cause production of the compound of interest by the microbial host cell, obtaining one or more samples of the liquid cell culture medium at periodic intervals and measuring the concentration of the compound of interest in each sample to determine the protease activity of the microbial host cell. The method may further comprise carrying out the same method on a test microbial host cell that has not been modified (i.e. a parent microbial host cell), with the exception of the introduction of the nucleotide sequence coding for the compound of interest, and comparing the concentration of the compound of interest in the cell culture medium with the concentration of the compound of interest in the cell culture medium for the modified microbial host cell to quantify a change in protease activity caused by the modification to the microbial host cell. Similar methods can be used to determine the yield of the compound of interest, for example culturing the microbial host cell in a cell culture medium under conditions to cause production of the compound of interest by the microbial host cell, obtaining one or more samples of the liquid cell culture medium at periodic intervals and measuring the concentration of the compound of interest in each sample to determine the yield of the compound of interest. The method may further comprise carrying out the same method on a test microbial host cell that has not been modified (i.e. a parent microbial host cell), with the exception of the introduction of the nucleotide sequence coding for the compound of interest, and comparing the concentration of the compound of interest in the cell culture medium with the concentration of the compound of interest in the cell culture medium for the modified microbial host cell to quantify a change in yield of the compound of interest caused by the modification to the microbial host cell.

In some embodiments obtaining a sample of the culture broth can include the step of removing the microbial host cell before obtaining a sample, or a sample of the culture broth can contain both the culture broth as the microbial host cell, or the microbial host cell can be lysed prior to taking a sample of the culture broth.

As the activity of a protease occurs over a period of time, when making comparisons in the protease activity between modified and parental microbial host cells, the skilled person will be aware the comparison may be made using protease activity measurement determined after the same culture time (i.e. after the modified and parental microbial host cells have been cultured for the same length of time). In addition, or as an alternative, the skilled person will be aware that the comparisons may be made using protease activity measurements from cultures that contain a similar amount of the microbial host cells. The skilled person will be aware that the comparison may be made using protease activity measurements starting from samples containing similar amounts of the microbial host cell (i.e. by making appropriate dilutions or concentrating samples before measurements). Similarly, when making comparisons in the yield of the compound of interest produced by modified and parental microbial host cells, the skilled person will be aware the comparison may be made using compound yield determined after the same culture time and/or starting from samples with the same amount of microbial host cell. This is simply an extension of the concept of measuring the protease activity and/or the compound yield under the same or substantially the same conditions for both the modified microbial host cell and the parental microbial host cell, and the skilled person would understand how to compare the protease activity between the modified and parental microbial host cells in this way.

A “parent microbial host cell” or “parental microbial host cell” is defined as a microbial host cell that has not been modified to affect the production, stability and/or function of the at least one polypeptide (and hence may be referred to as an unmodified microbial host cell). The parent microbial host cell therefore lacks one or more genetic modifications that affect the production, stability and/or function of the at least one polypeptide, and/or the parent microbial host cell is not subjected to an inhibiting compound or composition, wherein the inhibiting compound or composition affects the production, stability and/or function of the at least one polypeptide. The parent microbial host cell will generally be genetically identical to the modified microbial host cell, with the exception of the modification (if the modification is a genetic modification) and optionally the presence in the modified microbial host cell of at least one polynucleotide coding for a compound of interest (if such a polynucleotide is present). The parent microbial host cell may therefore be considered a wild-type host cell (and is referred to herein as such), since the host cell has not been modified to affect the production, stability and/or function of the at least one polypeptide. Generally therefore, a parent microbial host cell will not have been modified to cause a reduction or deficiency in protease activity.

In some embodiments, the parent host cell has not been modified in the same way as the modified host cell. In other words, the parent host cell may have undergone modification, but it has not undergone the modification to affect the production, stability and/or function of the at least one polypeptide. Thus, the parent host cell does not have a modulation in protease activity.

A “microbial host cell which has been modified” or a “modified microbial host cell” is herewith defined as a microbial host cell derived from a parent host cell and which has been modified, to obtain a different genotype and/or a different phenotype if compared to the parent host cell from which it is derived. The modification can either be affected by, for example:
a. subjecting the parent microbial host cell to recombinant genetic manipulation techniques;
b. subjecting the parent microbial host cell to (classical) mutagenesis; and/or
c. subjecting the parent microbial host cell to an inhibiting compound or composition.

In preferred embodiments, the modification may be a genetic modification.

A “modification affecting the production, stability and/or function of a polypeptide” means that the polypeptide, such as a polypeptide according to SEQ ID NOS: 1, 28, 33, 36, 58 or 59, is modulated in its activity and/or its intracellular and/or extracellular concentration is modulated when compared to the parent host cell and measured under the same or substantially the same conditions. “Production” of the polypeptide refers to production of the polypeptide by the microbial host cell. “Stability and/or function” of the polypeptide refers to the stability and/or function of the polypeptide inside or outside the microbial cell. The polypeptide whose production, stability and/or function is being modified is a polypeptide directly or indirectly involved in the expression or activity of one or more proteases in the modified microbial host cell. Accordingly, the modification of the polypeptide causes the modulation in the expression or activity of the one or more proteases. For example, the polypeptide whose production, stability and/or function is being modified may be a polypeptide that controls the expression of one or more proteases. The polypeptide may control the expression of one or more proteases by controlling the rate of transcription of one or more proteases (direct control), or the polypeptide may control the rate of transcription of one or more genes that in turn affect the expression of one or more proteases in the microbial host cell (indirect control).

For example, in preferred embodiments, the polypeptide may be a regulator of transcription, in particular a regulator of transcription that regulates the transcription of one or more protease genes encoded by the microbial host cell genome. As used herein, a “regulator of transcription” is a protein that regulates transcription, i.e. a polypeptide that causes, promotes, initiates, interrupts, represses or halts the transcription (and hence expression) of one or more genes (for example one or more genes coding for one or more proteases encoded by the microbial host cell genome). Generally, this is achieved by binding of the regulator of transcription to a specific DNA region (through use of a DNA binding domain), usually on or in the vicinity of one or more promoters, but essentially having its effect on the activity of said promoter or promoters, the genes being under the control of the promoters. For example, the proteases whose expression and/or activity is modulated may be under the control of the regulator of transcription. “Under the control” of the regulator of transcription means the regulator of transcription may directly control the rate of transcription of the relevant gene or genes (the one or more protease genes). Alternatively or additionally, the regulator of transcription may indirectly control the rate of transcription of the relevant gene or genes (the one or more protease genes). For example, the regulator of transcription may directly control the rate of transcription of one or more genes that in turn directly or indirectly control the transcription of the relevant gene or genes (the one or more protease genes). The number of stages in the pathway may differ, but importantly, the one or more polypeptides whose production, stability and/or function is being modulated by the modification of the microbial host cell should preferably (directly or indirectly) affect the protease activity of the microbial host cell.

By modifying the production, stability and/or function of a regulator of transcription, for example a regulator of transcription that controls the activity of one or more protease genes, one can affect the protease activity of the modified microbial host cell. The protease activity of the microbial host cell may be considered the cumulative activity of one or more proteases expressed by the microbial host cell, for example during culture or fermentation. In some embodiments, the modified protease activity of the modified microbial host cell may be the protease activity of one or more protease genes under the control of the regulator of transcription whose production, stability and/or function has been modified.

The regulator of transcription may be a “promoter of transcription” or a “repressor of transcription repressor”. A “promoter of transcription” is a protein that causes, promotes or initiates the transcription (and hence expression) of one or more genes. A promoter of transcription may be considered an enhancer of transcription and the terms “promoter of transcription” and “enhancer of transcription” may be used interchangeably“. A “repressor of transcription” is a protein that interrupts, represses or halts the transcription (and hence expression) of one or more genes. The regulator of transcription may be modified to adversely affect the production, stability and/or function of the said regulator of transcription, i.e. modified to diminish or reduce in some way the production, stability and/or function of the said regulator of transcription, or the regulator of transcription may be modified to positively affect the production, stability and/or function of the said regulator of transcription, i.e. modified to enhance or increase in some way the production, stability and/or function of the said regulator of transcription. The choice of an adverse modification or a positive modification may depend on the type of regulator of transcription that may be modified. For example, if the regulator of transcription is a promoter of transcription, the promoter of transcription may be modified to adversely affect the production, stability and/or function of the said promoter of transcription, i.e. modified to diminish or reduce in some way the production, stability and/or function of the said promoter of transcription, to reduce the level of expression of the gene or genes under control of the promoter of transcription. Alternatively, if the regulator of transcription is a repressor of transcription, the repressor of transcription may be modified to positively affect the production, stability and/or function of the said promoter of transcription, i.e. modified to enhance or increase in some way the production, stability and/or function of the said repressor of transcription, to reduce the level of expression of the gene or genes under control of the promoter of transcription.

In some embodiments, the modification is one that adversely affects the production, stability and/or function of the said polypeptide, i.e. one which diminishes or reduces in some way the production, stability and/or function of the said polypeptide. For example, in some embodiments where a reduction in protease activity may be desired, the modification may adversely affect the production, stability and/or function of a promoter of transcription, in particular a promoter of transcription that (directly or indirectly) causes, promotes or initiates the expression of one or more proteases in the microbial host cell. Therefore, the adverse modification of the polypeptide causes a reduction in the activity and/or expression of one or more proteases. For example, in some embodiments where a reduction in protease activity may be desired, the modification may adversely affect the production, stability and/or function of a promoter of transcription, in particular a promoter of transcription that directly causes, promotes or initiates the expression of one or more proteases in the microbial host cell (direct control). In some embodiments where a reduction in protease activity may be desired, the modification may adversely affect the production, stability and/or function of a promoter of transcription, in particular a promoter of transcription that causes, promotes or initiates the expression of one or more genes that cause, promote or initiate the expression of one or more proteases in the microbial host cell (indirect control).

Alternatively, in other embodiments where a reduction in protease activity may be desired, the modification may positively affect the production, stability and/or function of a repressor of transcription, in particular a repressor of transcription that (directly or indirectly) interrupts, represses or halts the expression of one or more proteases in the microbial host cell. Therefore, the positive modification of the polypeptide causes a decrease in the activity and/or expression of one or more proteases. For example, in some embodiments where a reduction in protease activity may be desired, the modification may positively affect the production, stability and/or function of a repressor of transcription, in particular a repressor of transcription that directly interrupts, represses or halts the expression of one or more proteases in the microbial host cell (direct control). In some embodiments where a reduction in protease activity may be desired, the modification may positively affect the production, stability and/or function of a repressor of transcription, in particular a repressor of transcription that interrupts, represses or halts the expression of one or more genes that promote the expression of one or more proteases in the microbial host cell (indirect control).

References herein to modifications that “positively affect” the production, stability and/or function of a polypeptide refer to modifications that increase the production, stability and/or function of the polypeptide. References herein to modifications that “adversely affect” the production, stability and/or function of a polypeptide refer to modifications that decrease the production, stability and/or function of the polypeptide. In some cases, the polypeptide may act as both a promoter and a repressor of transcription. For example, the polypeptide may act as a promoter of protease expression. The polypeptide may also act as a repressor of expression, or example of other proteins, for example cellobiohydrolase expression.

The polypeptide whose production, stability and/or function is being affected is a polypeptide expressed by a gene that is contained within the genome of a parental or wild-type microbial host cell. In other words, the polypeptide is not a heterologous polypeptide. Instead it is a polypeptide that is coded for by the genome of a parental or wild-type microbial host cell. After modification, the microbial host cell might no longer contain the gene that codes for or expresses the polypeptide, for example in the embodiments in which partial or full deletion of the gene occurs to adversely affect its production, stability and/or function. However, in some embodiments, the microbial host cell might still contain a full copy of the gene that codes for or expresses the polypeptide, for example in the embodiments in which the modification of the polypeptide is a positive modification, or in the embodiments in which the modification is one cause by administration of an inhibitor compounds (such as an RNAi or siRNA molecule that targets the gene encoding the polypeptide).

In some embodiments, the polypeptide whose production, structure and/or function is being modulated is a polypeptide having (comprising or consisting of) a sequence selected from the group consisting of SEQ ID NOs: 1, 28, 33, 36, 58 and 59. In some embodiments, the polypeptide may have an amino acid sequence that is at least about 80%, at least about 85%, at least about 90%, at least about 95%, or at least about 98% identical to a sequence selected from the group consisting of SEQ ID NOs: 1, 28, 33, 36, 58 and 59. In some embodiments, the polypeptide whose production, structure and/or function is being modulated is a polypeptide that is an ortholog of the polypeptide having (comprising or consisting of) a sequence selected from the group consisting of SEQ ID NOs: 1, 28, 33, 36, 58 and 59.

In some embodiments, the polypeptide whose production, structure and/or function is being modulated is a polypeptide encoded by a genomic nucleotide sequence having (comprising or consisting of) a sequence selected from the group consisting of SEQ ID NOs: 2, 29, 34 and 37. In some embodiments, the nucleotide sequence may have a sequence that is at least about 80%, at least about 85%, at least about 90%, at least about 95%, or at least about 98% identical to a sequence selected from the group consisting of SEQ ID NOS: 2, 29, 34 and 37. In some embodiments, the polypeptide whose production, structure and/or function is being modulated is a polypeptide that is an ortholog of the polypeptide encoded by a genomic nucleotide sequence having (comprising or consisting of) a sequence selected from the group consisting of SEQ ID NOs: 2, 29, 34 and 37.

In some embodiments, the polypeptide whose production, structure and/or function is being modulated is a polypeptide encoded by a nucleotide sequence having (comprising or consisting of) a sequence according to a sequence selected from the group consisting of SEQ ID NOs: 3, 30, 35 and 38. In some embodiments, the nucleotide sequence may have a sequence that is at least about 80%, at least about 85%, at least about 90%, at least about 95% identical, or at least about 98% identical to a sequence selected from the group consisting of SEQ ID NOS: 3, 30, 35 and 38. In some embodiments, the polypeptide whose production, structure and/or function is being modulated is a polypeptide that is an ortholog of the polypeptide encoded by a nucleotide sequence having (comprising or consisting of) a sequence according to a sequence selected from the group consisting of SEQ ID NOs: 3, 30, 35 and 38.

In some embodiments, the polypeptide whose production, structure and/or function is being modulated is a polypeptide having (comprising or consisting of) a sequence according to SEQ ID NO: 1. In some embodiments, the polypeptide may have an amino acid sequence that is at least about 80%, at least about 85%, at least about 90%, at least about 95%, or at least about 98% identical to SEQ ID NO: 1. In some embodiments, the polypeptide whose production, structure and/or function is being modulated is a polypeptide that is an ortholog of the polypeptide having (comprising or consisting of) a sequence according to SEQ ID NO: 1.

In some embodiments, the polypeptide whose production, structure and/or function is being modulated is a polypeptide encoded by a genomic nucleotide sequence having (comprising or consisting of) a sequence according to SEQ ID NO: 2. In some embodiments, the nucleotide sequence may have a sequence that is at least about 80%, at least about 85%, at least about 90%, at least about 95%, or at least about 98% identical to SEQ ID NO: 2. In some embodiments, the polypeptide whose production, structure and/or function is being modulated is a polypeptide that is an ortholog of the polypeptide encoded by a genomic nucleotide sequence having (comprising or consisting of) a sequence according to SEQ ID NO: 2.

In some embodiments, the polypeptide whose production, structure and/or function is being modulated is a polypeptide encoded by a nucleotide sequence having (comprising or consisting of) a sequence according to SEQ ID NO: 3. In some embodiments, the nucleotide sequence may have a sequence that is at least about 80%, at least about 85%, at least about 90%, at least about 95% identical, or at least about 98% identical to SEQ ID NO: 3. In some embodiments, the polypeptide whose production, structure and/or function is being modulated is a polypeptide that is an ortholog of the polypeptide encoded by a nucleotide sequence having (comprising or consisting of) a sequence according to SEQ ID NO: 3.

In some embodiments, the polypeptide whose production, structure and/or function is being modulated is a polypeptide having (comprising or consisting of) a sequence according to SEQ ID NO: 28. In some embodiments, the polypeptide may have an amino acid sequence that is at least about 80%, at least about 85%, at least about 90%, at least about 95%, or at least about 98% identical to SEQ ID NO: 28. In some embodiments, the polypeptide whose production, structure and/or function is being modulated is a polypeptide that is an ortholog of the polypeptide having (comprising or consisting of) a sequence according to SEQ ID NO: 28.

In some embodiments, the polypeptide whose production, structure and/or function is being modulated is a polypeptide encoded by a genomic nucleotide sequence having (comprising or consisting of) a sequence according to SEQ ID NO: 29. In some embodiments, the nucleotide sequence may have a sequence that is at least about 80%, at least about 85%, at least about 90%, at least about 95%, or at least about 98% identical to SEQ ID NO: 29. In some embodiments, the polypeptide whose production, structure and/or function is being modulated is a polypeptide that is an ortholog of the polypeptide encoded by a genomic nucleotide sequence having (comprising or consisting of) a sequence according to SEQ ID NO: 29.

In some embodiments, the polypeptide whose production, structure and/or function is being modulated is a polypeptide encoded by a nucleotide sequence having (comprising or consisting of) a sequence according to SEQ ID NO: 30. In some embodiments, the nucleotide sequence may have a sequence that is at least about 80%, at least about 85%, at least about 90%, at least about 95% identical, or at least about 98% identical to SEQ ID NO: 30. In some embodiments, the polypeptide whose production, structure and/or function is being modulated is a polypeptide that is an ortholog of the polypeptide encoded by a nucleotide sequence having (comprising or consisting of) a sequence according to SEQ ID NO: 30.

In some embodiments, the polypeptide whose production, structure and/or function is being modulated is a polypeptide having (comprising or consisting of) a sequence according to SEQ ID NO: 33.

In some embodiments, the polypeptide may have an amino acid sequence that is at least about 80%, at least about 85%, at least about 90%, at least about 95%, or at least about 98% identical to SEQ ID NO: 33. In some embodiments, the polypeptide whose production, structure and/or function is being modulated is a polypeptide that is an ortholog of the polypeptide having (comprising or consisting of) a sequence according to SEQ ID NO: 33.

In some embodiments, the polypeptide whose production, structure and/or function is being modulated is a polypeptide encoded by a genomic nucleotide sequence having (comprising or consisting of) a sequence according to SEQ ID NO: 34. In some embodiments, the nucleotide sequence may have a sequence that is at least about 80%, at least about 85%, at least about 90%, at least about 95%, or at least about 98% identical to SEQ ID NO: 34. In some embodiments, the polypeptide whose production, structure and/or function is being modulated is a polypeptide that is an ortholog of the polypeptide encoded by a genomic nucleotide sequence having (comprising or consisting of) a sequence according to SEQ ID NO: 34.

In some embodiments, the polypeptide whose production, structure and/or function is being modulated is a polypeptide encoded by a nucleotide sequence having (comprising or consisting of) a sequence according to SEQ ID NO: 35. In some embodiments, the nucleotide sequence may have a sequence that is at least about 80%, at least about 85%, at least about 90%, at least about 95% identical, or at least about 98% identical to SEQ ID NO: 35. In some embodiments, the polypeptide whose production, structure and/or function is being modulated is a polypeptide that is an ortholog of the polypeptide encoded by a nucleotide sequence having (comprising or consisting of) a sequence according to SEQ ID NO: 35.

In some embodiments, the polypeptide whose production, structure and/or function is being modulated is a polypeptide having (comprising or consisting of) a sequence according to SEQ ID NO: 36. In some embodiments, the polypeptide may have an amino acid sequence that is at least about 80%, at least about 85%, at least about 90%, at least about 95%, or at least about 98% identical to SEQ ID NO: 36. In some embodiments, the polypeptide whose production, structure and/or function is being modulated is a polypeptide that is an ortholog of the polypeptide having (comprising or consisting of) a sequence according to SEQ ID NO: 36.

In some embodiments, the polypeptide whose production, structure and/or function is being modulated is a polypeptide encoded by a genomic nucleotide sequence having (comprising or consisting of) a sequence according to SEQ ID NO: 37. In some embodiments, the nucleotide sequence may have a sequence that is at least about 80%, at least about 85%, at least about 90%, at least about 95%, or at least about 98% identical to SEQ ID NO: 37. In some embodiments, the polypeptide whose production, structure and/or function is being modulated is a polypeptide that is an ortholog of the polypeptide encoded by a genomic nucleotide sequence having (comprising or consisting of) a sequence according to SEQ ID NO: 37.

In some embodiments, the polypeptide whose production, structure and/or function is being modulated is a polypeptide encoded by a nucleotide sequence having (comprising or consisting of) a sequence according to SEQ ID NO: 38. In some embodiments, the nucleotide sequence may have a sequence that is at least about 80%, at least about 85%, at least about 90%, at least about 95% identical, or at least about 98% identical to SEQ ID NO: 38. In some embodiments, the polypeptide whose production, structure and/or function is being modulated is a polypeptide that is an ortholog of the polypeptide encoded by a nucleotide sequence having (comprising or consisting of) a sequence according to SEQ ID NO: 38.

In some embodiments, the polypeptide whose production, structure and/or function is being modulated is a polypeptide having (comprising or consisting of) a sequence according to SEQ ID NO: 58. In some embodiments, the polypeptide may have an amino acid sequence that is at least about 80%, at least about 85%, at least about 90%, at least about 95%, or at least about 98% identical to SEQ ID NO: 58. In some embodiments, the polypeptide whose production, structure and/or function is being modulated is a polypeptide that is an ortholog of the polypeptide having (comprising or consisting of) a sequence according to SEQ ID NO: 58.

In some embodiments, the polypeptide whose production, structure and/or function is being modulated is a polypeptide having (comprising or consisting of) a sequence according to SEQ ID NO: 59. In some embodiments, the polypeptide may have an amino acid sequence that is at least about 80%, at least about 85%, at least about 90%, at least about 95%, or at least about 98% identical to SEQ ID NO: 59. In some embodiments, the polypeptide whose production, structure and/or function is being modulated is a polypeptide that is an ortholog of the polypeptide having (comprising or consisting of) a sequence according to SEQ ID NO: 59.

In some embodiments, the host cell may be modified to affect the production, stability and/or function of at least one polypeptide comprising a sequence selected from the group consisting of SEQ ID NOs: 1, 28, 33, 36, 58 and 59 or a polypeptide at least about 80%, at least about 85%, at least about 90%, at least about 95%, or at least about 98% identical thereto, or an ortholog thereof, and wherein the microbial host cell has been further modified to affect the production, stability and/or function of one or more additional polypeptides. In such embodiments, the modification of a plurality of polypeptides may have beneficial effects, such as an increase in yields in embodiments related to the production of a compound of interest. The additional polypeptides that may be modified in such approaches may have any of the preferred or more specific features of the modified polypeptides as described herein.

When amino acid or nucleotide sequences are used having a defined percent identity, they will generally still retain the function of the full-length reference sequence.

For example, the host cell may be modified to affect the production, stability and/or function of at least one polypeptide comprising a sequence selected from the group consisting of SEQ ID NOs: 1, 28, 33, 36, 58 and 59 or a functional variant thereof, wherein a functional variant is a variant having at least about 80%, at least about 85%, at least about 90%, at least about 95%, or at least about 98% identity to a sequence selected from the group consisting of SEQ ID NOS: 1, 28, 33, 36, 58 and 59, wherein the functional variant retains the same function as the polypeptide having an amino acid sequence that is 100% identical to a sequence selected from the group consisting of SEQ ID NOs: 1, 28, 33, 36, 58 and 59.

In embodiments where the polypeptide is a regulator of transcription (as is the case for a polypeptide having a sequence selected from the group consisting of SEQ ID NOS: 1, 28, 33, 36, 58 and 59), the functional variants (i.e. those having a certain percent identity relative to a sequence selected from the group consisting of SEQ ID NOS: 1, 28, 33, 36, 58 and 59) may retain the regulation of transcription function. More specifically, the functional variants may retain the ability to control (directly or indirectly) the same one or more genes (such as protease genes) whose transcription is/are controlled by a polypeptide having a sequence selected from the group consisting of SEQ ID NOS: 1, 28, 33, 36, 58 and 59.

When the polypeptide is a promoter of transcription, the functional variants of the promoter of transcription may retain the promoter of transcription function. More specifically, the functional variants may retain the ability to cause, promote or initiate (directly or indirectly) the same one or more genes (such as protease genes) whose transcription is/are caused, promoted or initiated by a promoter of transcription having a sequence selected from the group consisting of SEQ ID NOs: 1, 28, 33, 36, 58 and 59. When the polypeptide is a repressor of transcription, the functional variants of the repressor of transcription may retain the repressor of transcription function. More specifically, the functional variants may retain the ability to interrupt, repress or halt (directly or indirectly) the same one or more genes (such as protease genes) whose transcription is/are interrupted, repressed or halted by a repressor of transcription having a sequence selected from the group consisting of SEQ ID NOS: 1, 28, 33, 36, 58 and 59.

The functional variants of polypeptides encoded by a sequence selected from the group consisting of SEQ ID NOS: 2, 3, 29, 30, 34, 35, 37 and 38 may retain their function in the same way as described above for variants of polypeptides having a sequence selected from the group consisting of SEQ ID NOS: 1, 28, 33 and 36.

In some embodiments, the polypeptide whose production, structure and/or function is being modulated is an ortholog of the target polypeptide, i.e. an ortholog of the polypeptide having an amino acid sequence selected from the group consisting of SEQ ID NOs: 1, 28, 33, 36, 58 and 59 (or an ortholog of a polypeptide encoded by a genomic nucleotide sequence comprising a sequence selected from the group consisting of SEQ ID NOs: 2, 29, 34 and 37 or a nucleotide sequence comprising a sequence selected from the group consisting of SEQ ID NO: 3, 30, 35 and 38).

An ortholog refers to any of two or more homologous gene or protein sequences found in different species related by linear descent. The orthologs serve the same or similar function in a different species. In some embodiments, the ortholog may be from a different genus. In some embodiments, the ortholog may be from the same genus. In some embodiments, the orthologs may be from the same species, but a different strain.

Preferably, the ortholog performs the same or similar function as the reference polypeptide. For example, in embodiments where the reference polypeptide is a regulator of transcription (as is the case for a polypeptide having a sequence selected from the group consisting of SEQ ID NOs: 1, 28, 33, 36, 58 and 59), the ortholog is also a regulator of transcription. More specifically, the ortholog may retain the ability to control (directly or indirectly) the same (or similar) one or more genes (such as protease genes, or orthologs thereof) whose transcription is/are controlled by the reference polypeptide having a sequence selected from the group consisting of SEQ ID NOS: 1, 28, 33, 36, 58 and 59. When the reference polypeptide is a promoter of transcription, the ortholog may also be a promoter of transcription. More specifically, the ortholog may retain the ability to cause, promote or initiate (directly or indirectly) the same (or similar) one or more genes (such as protease genes, or orthologs thereof)) whose transcription is/are caused, promoted or initiated by the reference promoter of transcription having a sequence selected from the group consisting of SEQ ID NOS: 1, 28, 33, 36, 58 and 59.

The orthologs of polypeptides encoded by a sequence selected from the group consisting of SEQ ID

NOs: 2, 3, 29, 30, 3, 35, 37 and 38 may perform the same function as described above for orthologs of polypeptides having a sequence selected from the group consisting of SEQ ID NOs: 1, 28, 33 and 36.

Orthologs may have sequence identity with one another. For example, in some embodiments, the orthologs may have at least about 35% identity, at least about 40% identity, at least about 50% identity, at least about 60% identity, at least about 70% identity, at least about 80% identity, at least about 90% identity, at least about 95% identity, at least about 96% identity, at least about 97% identity, at least about 98% identity, or at least about 99% identity across their length with the polypeptide whose production, structure and/or function is being modulated. In some embodiments, the orthologs may have at least about 35% identity, at least about 40% identity, at least about 50% identity, at least about 60% identity, at least about 70% identity, at least about 80% identity, at least about 90% identity, at least about 95% identity, at least about 96% identity, at least about 97% identity, at least about 98% identity, or at least about 99% identity with the polypeptide whose production, structure and/or function is being modulated and they may be from about 80% to about 120% the length of the polypeptide whose production, structure and/or function is being modulated. In some embodiments, the orthologs may have at least about 35% identity with the polypeptide whose production, structure and/or function is being modulated and they may be from about 80% to about 120% the length of the polypeptide whose production, structure and/or function is being modulated. In some embodiments, the orthologs may be from the same genus and have at least about 90% identity with the polypeptide whose production, structure and/or function is being modulated and they may be from about 80% to about 120% the length of the polypeptide whose production, structure and/or function is being modulated.

Orthologs may comprise conserved sequences. For example, in some embodiments, an ortholog comprises a sequence having at least about 95% sequence identity with SEQ ID NO: 31. In some embodiments, an ortholog comprises the sequence of SEQ ID NO: 31.

In some embodiments, an ortholog of the polypeptide of SEQ ID NO: 1 may comprise a sequence having at least about 95% sequence identity with the amino acids 685-735 of SEQ ID NO: 1. In some embodiments, an ortholog of the polypeptide of SEQ ID NO: 1 may comprise a sequence comprising amino acids 685-735 of SEQ ID NO: 1. In some embodiments, an ortholog of the polypeptide of SEQ ID NO: 28 may comprise a sequence having at least about 95% sequence identity with the amino acids 753-803 of SEQ ID NO: 28. In some embodiments, an ortholog of the polypeptide of SEQ ID NO: 28 may comprise a sequence comprising amino acids 753-803 of SEQ ID NO: 28. In some embodiments, an ortholog of the polypeptide of SEQ ID NO: 33 may comprise a sequence having at least about 95% sequence identity with the amino acids 749-799 of SEQ ID NO: 33. In some embodiments, an ortholog of the polypeptide of SEQ ID NO: 33 may comprise a sequence comprising amino acids 749-799 of SEQ ID NO: 33. In some embodiments, an ortholog of the polypeptide of SEQ ID NO: 36 may comprise a sequence having at least about 95% sequence identity with the amino acids 670-720 of SEQ ID NO: 36. In some embodiments, an ortholog of the polypeptide of SEQ ID NO: 36 may comprise a sequence comprising amino acids 670-720 of SEQ ID NO: 36. In some embodiments, an ortholog of the polypeptide of SEQ ID NO: 58 may comprise a sequence having at least about 95% sequence identity with the amino acids 679-729 of SEQ ID NO: 58.

In some embodiments, an ortholog of the polypeptide of SEQ ID NO: 58 may comprise a sequence comprising amino acids 679-729 of SEQ ID NO: 58. In some embodiments, an ortholog of the polypeptide of SEQ ID NO: 59 may comprise a sequence having at least about 95% sequence identity with the amino acids 749-799 of SEQ ID NO: 59. In some embodiments, an ortholog of the polypeptide of SEQ ID NO: 59 may comprise a sequence comprising amino acids 749-799 of SEQ ID NO: 59.

The orthologs may additionally comprise a defined sequence identity to a longer reference sequence. For example, in some embodiments, the ortholog may have at least about 35%, at least about 40%, at least about 45%, at least about 50%, at least about 60%, at least about 70%, at least about 80%, at least about 90%, at least about 95%, at least about 96%, at least about 97%, at least about 98% or at least about 99% sequence identity to the reference sequence, in addition to comprising a highly conserved sequence. For example, in some embodiments, an ortholog of the polypeptide of SEQ ID NO: 1 may comprise a sequence having at least about 95% sequence identity with the amino acids 685-735 of SEQ ID NO: 1 and the ortholog may comprise a sequence that is at least 35% identical to the sequence of SEQ ID NO: 1. In some embodiments, an ortholog of the polypeptide of SEQ ID NO: 28 may comprise a sequence having at least about 95% sequence identity with the amino acids 753-803 of SEQ ID NO: 28 and the ortholog may comprise a sequence that is at least 35% identical to the sequence of SEQ ID NO: 28. In some embodiments, an ortholog of the polypeptide of SEQ ID NO: 33 may comprise a sequence having at least about 95% sequence identity with the amino acids 749-799 of SEQ ID NO: 33 and the ortholog may comprise a sequence that is at least 35% identical to the sequence of SEQ ID NO: 33. In some embodiments, an ortholog of the polypeptide of SEQ ID NO: 36 may comprise a sequence having at least about 95% sequence identity with the amino acids 670-720 of SEQ ID NO: 36 and the ortholog may comprise a sequence that is at least 35% identical to the sequence of SEQ ID NO: 36. In some embodiments, an ortholog of the polypeptide of SEQ ID NO: 58 may comprise a sequence having at least about 95% sequence identity with the amino acids 679-729 of SEQ ID NO: 58 and the ortholog may comprise a sequence that is at least 35% identical to the sequence of SEQ ID NO: 58. In some embodiments, an ortholog of the polypeptide of SEQ ID NO: 59 may comprise a sequence having at least about 95% sequence identity with the amino acids 749-799 of SEQ ID NO: 59 and the ortholog may comprise a sequence that is at least 35% identical to the sequence of SEQ ID NO: 59.

In some embodiments, an ortholog of the polypeptide of SEQ ID NO: 1 may comprise a sequence having amino acids 685-735 of SEQ ID NO: 1 and the ortholog may comprise a sequence that is at least 35% identical to the sequence of SEQ ID NO: 1. In some embodiments, an ortholog of the polypeptide of SEQ ID NO: 28 may comprise a sequence having amino acids 753-803 of SEQ ID NO: 28 and the ortholog may comprise a sequence that is at least 35% identical to the sequence of SEQ ID NO: 28. In some embodiments, an ortholog of the polypeptide of SEQ ID NO: 33 may comprise a sequence having amino acids 749-799 of SEQ ID NO: 33 and the ortholog may comprise a sequence that is at least 35% identical to the sequence of SEQ ID NO: 33. In some embodiments, an ortholog of the polypeptide of SEQ ID NO: 36 may comprise a sequence having amino acids 670-720 of SEQ ID NO: 36 and the ortholog may comprise a sequence that is at least 35% identical to the sequence of SEQ ID NO: 36. In some embodiments, an ortholog of the polypeptide of SEQ ID NO: 58 may comprise a sequence having amino acids 679-729 of SEQ ID NO: 58 and the ortholog may comprise a sequence that is at least 35% identical to the sequence of SEQ ID NO: 58. In some embodiments, an ortholog of the polypeptide of SEQ ID NO:

59 may comprise a sequence having amino acids 749-799 of SEQ ID NO: 59 and the ortholog may comprise a sequence that is at least 35% identical to the sequence of SEQ ID NO: 59.

In some embodiments, variations in sequence between an ortholog and a reference sequence may be conservative variations. For example, in some embodiments, when the sequences of an ortholog having at least 35% identity to SEQ ID NO: 1 and comprising a sequence having at least about 95% sequence identity with the amino acids 685-735 of SEQ ID NO: 1 is aligned with the sequence of SEQ ID NO: 1, at least 50% of the variations between the ortholog and the sequence of SEQ ID NO: 1 are conservative amino acid substitutions. In some embodiments, at least 60%, at least 70%, at least 80% or at least 90% of the variations between the ortholog and the sequence of SEQ ID NO: 1 are conservative amino acid substitutions. In some embodiments, when the sequences of an ortholog having at least 35% identity to SEQ ID NO: 28 and comprising a sequence having at least about 95% sequence identity with the amino acids 753-803 of SEQ ID NO: 28 is aligned with the sequence of SEQ ID NO: 28, at least 50% of the variations between the ortholog and the sequence of SEQ ID NO: 28 are conservative amino acid substitutions. In some embodiments, at least 60%, at least 70%, at least 80% or at least 90% of the variations between the ortholog and the sequence of SEQ ID NO: 28 are conservative amino acid substitutions. In some embodiments, when the sequences of an ortholog having at least 35% identity to SEQ ID NO: 33 and comprising a sequence having at least about 95% sequence identity with the amino acids 749-799 of SEQ ID NO: 33 is aligned with the sequence of SEQ ID NO: 33, at least 50% of the variations between the ortholog and the sequence of SEQ ID NO: 33 are conservative amino acid substitutions. In some embodiments, at least 60%, at least 70%, at least 80% or at least 90% of the variations between the ortholog and the sequence of SEQ ID NO: 33 are conservative amino acid substitutions. In some embodiments, when the sequences of an ortholog having at least 35% identity to SEQ ID NO: 36 and comprising a sequence having at least about 95% sequence identity with the amino acids 670-720 of SEQ ID NO: 36 is aligned with the sequence of SEQ ID NO: 36, at least 50% of the variations between the ortholog and the sequence of SEQ ID NO: 36 are conservative amino acid substitutions. In some embodiments, at least 60%, at least 70%, at least 80% or at least 90% of the variations between the ortholog and the sequence of SEQ ID NO: 36 are conservative amino acid substitutions. In some embodiments, when the sequences of an ortholog having at least 35% identity to SEQ ID NO: 58 and comprising a sequence having at least about 95% sequence identity with the amino acids 679-729 of SEQ ID NO: 58 is aligned with the sequence of SEQ ID NO: 58, at least 50% of the variations between the ortholog and the sequence of SEQ ID NO: 58 are conservative amino acid substitutions. In some embodiments, at least 60%, at least 70%, at least 80% or at least 90% of the variations between the ortholog and the sequence of SEQ ID NO: 58 are conservative amino acid substitutions. In some embodiments, when the sequences of an ortholog having at least 35% identity to SEQ ID NO: 59 and comprising a sequence having at least about 95% sequence identity with the amino acids 749-799 of SEQ ID NO: 59 is aligned with the sequence of SEQ ID NO: 59, at least 50% of the variations between the ortholog and the sequence of SEQ ID NO: 59 are conservative amino acid substitutions. In some embodiments, at least 60%, at least 70%, at least 80% or at least 90% of the variations between the ortholog and the sequence of SEQ ID NO: 59 are conservative amino acid substitutions.

In some embodiments, when the sequences of an ortholog having at least 35% identity to SEQ ID NO: 1 and comprising amino acids 685-735 of SEQ ID NO: 1 is aligned with the sequence of SEQ ID NO:
1, at least 50% of the variations between the ortholog and the sequence of SEQ ID NO: 1 are conservative amino acid substitutions. In some embodiments, at least 60%, at least 70%, at least 80% or at least 90% of the variations between the ortholog and the sequence of SEQ ID NO: 1 are conservative amino acid substitutions. In some embodiments, when the sequences of an ortholog having at least 35% identity to SEQ ID NO: 28 and comprising amino acids 753-803 of SEQ ID NO: 28 is aligned with the sequence of SEQ ID NO: 28, at least 50% of the variations between the ortholog and the sequence of SEQ ID NO: 28 are conservative amino acid substitutions. In some embodiments, at least 60%, at least 70%, at least 80% or at least 90% of the variations between the ortholog and the sequence of SEQ ID NO: 28 are conservative amino acid substitutions. In some embodiments, when the sequences of an ortholog having at least 35% identity to SEQ ID NO: 33 and comprising amino acids 749-799 of SEQ ID NO: 33 is aligned with the sequence of SEQ ID NO: 33, at least 50% of the variations between the ortholog and the sequence of SEQ ID NO: 33 are conservative amino acid substitutions. In some embodiments, at least 60%, at least 70%, at least 80% or at least 90% of the variations between the ortholog and the sequence of SEQ ID NO: 33 are conservative amino acid substitutions. In some embodiments, when the sequences of an ortholog having at least 35% identity to SEQ ID NO: 36 and comprising amino acids 670-720 of SEQ ID NO: 36 is aligned with the sequence of SEQ ID NO: 36, at least 50% of the variations between the ortholog and the sequence of SEQ ID NO: 36 are conservative amino acid substitutions. In some embodiments, at least 60%, at least 70%, at least 80% or at least 90% of the variations between the ortholog and the sequence of SEQ ID NO: 36 are conservative amino acid substitutions. In some embodiments, when the sequences of an ortholog having at least 35% identity to SEQ ID NO: 58 and comprising amino acids 679-729 of SEQ ID NO: 58 is aligned with the sequence of SEQ ID NO: 58, at least 50% of the variations between the ortholog and the sequence of SEQ ID NO: 58 are conservative amino acid substitutions. In some embodiments, at least 60%, at least 70%, at least 80% or at least 90% of the variations between the ortholog and the sequence of SEQ ID NO: 58 are conservative amino acid substitutions. In some embodiments, when the sequences of an ortholog having at least 35% identity to SEQ ID NO: 59 and comprising amino acids 749-799 of SEQ ID NO: 59 is aligned with the sequence of SEQ ID NO: 59, at least 50% of the variations between the ortholog and the sequence of SEQ ID NO: 59 are conservative amino acid substitutions. In some embodiments, at least 60%, at least 70%, at least 80% or at least 90% of the variations between the ortholog and the sequence of SEQ ID NO: 59 are conservative amino acid substitutions.

Conservative amino acid substitutions are well known to the person of skill in the art. For example, conservative amino acid substitutions may be:
a) the substitution of any glycine, alanine, valine, leucine or isoleucine residues in the reference sequence with another amino acid selected from glycine, alanine, valine, leucine and isoleucine;
b) the substitution of any serine, cysteine, threonine or methionine residues in the reference sequence with another amino acid selected from serine, cysteine, threonine and methionine;
c) the substitution of any phenylalanine, tyrosine or tryptophan residues in the reference sequence with another amino acid selected from phenylalanine, tyrosine and tryptophan;
d) the substitution of any histidine, lysine or arginine residues in the reference sequence with another amino acid selected from histidine, lysine or arginine; and
e) the substitution of any aspartate, glutamate, asparagine or glutamine residues in the reference sequence with another amino acid selected from aspartate, glutamate, asparagine and glutamine.

In some embodiments, the ortholog may be from a Trichoderma spp., a Myceliophthora spp., an Aspergillus spp., a Penicillium spp., a Rasamsonia spp. or a Fusarium spp. In some embodiments, the ortholog may be from a Trichoderma spp., a Myceliophthora spp . . . or an Aspergillus spp. . . . In some embodiments, the ortholog may be from a Trichoderma spp. or a Myceliophthora spp. In some embodiments, for example, but not limited to, embodiments relating to SEQ ID NOS: 1 to 3, the ortholog may be from a Trichoderma spp. . . . In some embodiments, for example, but not limited to, embodiments relating to SEQ ID NO: 28 to 30 or 33 to 35, the ortholog may be from a Myceliophthora spp. . . . In some embodiments, for example, but not limited to, embodiments relating to SEQ ID NO: 36 to 38, the ortholog may be from a Aspergillus spp. . . . However, as noted above, orthologs may not necessarily be from the same genus as the reference polypeptide.

In some embodiments, the polypeptide whose production, structure and/or function is being modulated (or ortholog thereof) comprises a GATA-type zinc finger domain. GATA-type zinc finger domains bind the DNA sequence X₁GATAX₂ (SEQ ID NO: 32), wherein X₁ is A or T and X₂ is A or G. In some embodiments, the polypeptide whose production, structure and/or function is being modulated (or ortholog thereof) does not comprise more than one GATA-type zinc finger domain

In some embodiments, the GATA-type zinc finger domain comprises a sequence having at least about 95% identity to SEQ ID NO: 31. In some embodiments, the GATA-type zinc finger domain comprises a sequence having at least about 96%, at least about 97%, at least about 98% or at least about 99% identity to SEQ ID NO: 31. In some embodiments, the GATA-type zinc finger domain comprises the sequence of SEQ ID NO: 31.

Generally, the polypeptide whose production, structure and/or function is being modulated (or ortholog thereof) is a fungal protein, i.e. a protein expressed by fungi. The protein may be a protein that is found in wild-type fungi, i.e. naturally occurring fungal species. In preferred embodiments, the polypeptide whose production, structure and/or function is being modulated (or ortholog thereof) is a filamentous fungal protein (i.e. a protein from filamentous fungi).

In some embodiments, the polypeptide whose production, structure and/or function is being modulated (or ortholog thereof) is a GATA-type transcriptional activator. In some embodiments, the polypeptide whose production, structure and/or function is being modulated (or ortholog thereof) is an Are polypeptide. In some embodiments, the polypeptide whose production, structure and/or function is being modulated (or ortholog thereof) is Are1 or AreA, or an ortholog of Are1 or AreA.

In some embodiments, the polypeptide whose production, structure and/or function is being modulated (or ortholog thereof) comprises a sequence having at least about 95% sequence identity with the amino acids 685-735 of SEQ ID NO: 1. In some embodiments, polypeptide whose production, structure and/or function is being modulated (or ortholog thereof) comprises a sequence comprising amino acids 685-735 of SEQ ID NO: 1. In some embodiments, polypeptide whose production, structure and/or function is being modulated (or ortholog thereof) comprises a sequence having at least about 95% sequence identity with the amino acids 753-803 of SEQ ID NO: 28. In some embodiments, polypeptide whose production, structure and/or function is being modulated (or ortholog thereof) comprises a sequence comprising amino acids 753-803 of SEQ ID NO: 28. In some embodiments, polypeptide whose production, structure and/or function is being modulated (or ortholog thereof) comprises a sequence having at least about 95% sequence identity with the amino acids 749-799 of SEQ ID NO: 33. In some embodiments, polypeptide whose production, structure and/or function is being modulated (or ortholog thereof) comprises a sequence comprising amino acids 749-799 of SEQ ID NO: 33. In some embodiments, polypeptide whose production, structure and/or function is being modulated (or ortholog thereof) comprises a sequence having at least about 95% sequence identity with the amino acids 670-720 of SEQ ID NO: 36. In some embodiments, polypeptide whose production, structure and/or function is being modulated (or ortholog thereof) comprises a sequence comprising amino acids 670-720 of SEQ ID NO: 36. In some embodiments, polypeptide whose production, structure and/or function is being modulated (or ortholog thereof) comprises a sequence having at least about 95% sequence identity with the amino acids 679-729 of SEQ ID NO: 58. In some embodiments, polypeptide whose production, structure and/or function is being modulated (or ortholog thereof) comprises a sequence comprising amino acids 679-729 of SEQ ID NO: 58. In some embodiments, polypeptide whose production, structure and/or function is being modulated (or ortholog thereof) comprises a sequence having at least about 95% sequence identity with the amino acids 749-799 of SEQ ID NO: 59.

In some embodiments, polypeptide whose production, structure and/or function is being modulated (or ortholog thereof) comprises a sequence comprising amino acids 749-799 of SEQ ID NO: 59.

In some embodiments, the polypeptide whose production, structure and/or function is being modulated (or ortholog thereof) comprises a sequence having at least about 95% sequence identity with the amino acids 685-735 of SEQ ID NO: 1 and the polypeptide may comprise a sequence that is at least about 35% identical to the sequence of SEQ ID NO: 1. In some embodiments, the polypeptide whose production, structure and/or function is being modulated (or ortholog thereof) comprises a sequence having at least about 95% sequence identity with the amino acids 753-803 of SEQ ID NO: 28 and the polypeptide may comprise a sequence that is at least about 35% identical to the sequence of SEQ ID NO: 28. In some embodiments, the polypeptide whose production, structure and/or function is being modulated (or ortholog thereof) comprises a sequence having at least about 95% sequence identity with the amino acids 749-799 of SEQ ID NO: 33 and the polypeptide may comprise a sequence that is at least about 35% identical to the sequence of SEQ ID NO: 33. In some embodiments, the polypeptide whose production, structure and/or function is being modulated (or ortholog thereof) comprises a sequence having at least about 95% sequence identity with the amino acids 670-720 of SEQ ID NO: 36 and the polypeptide may comprise a sequence that is at least about 35% identical to the sequence of SEQ ID NO: 36. In some embodiments, the polypeptide whose production, structure and/or function is being modulated (or ortholog thereof) comprises a sequence having at least about 95% sequence identity with the amino acids 679-729 of SEQ ID NO: 58 and the polypeptide may comprise a sequence that is at least about 35% identical to the sequence of SEQ ID NO: 58. In some embodiments, the polypeptide whose production, structure and/or function is being modulated (or ortholog thereof) comprises a sequence having at least about 95% sequence identity with the amino acids 749-799 of SEQ ID NO: 59 and the polypeptide may comprise a sequence that is at least about 35% identical to the sequence of SEQ ID NO: 59.

In some embodiments, the polypeptide whose production, structure and/or function is being modulated (or ortholog thereof) comprises a sequence having amino acids 685-735 of SEQ ID NO: 1 and the polypeptide may comprise a sequence that is at least about 35% identical to the sequence of SEQ ID NO: 1. In some embodiments, the polypeptide whose production, structure and/or function is being modulated (or ortholog thereof) comprises a sequence having amino acids 753-803 of SEQ ID NO: 28 and the polypeptide may comprise a sequence that is at least about 35% identical to the sequence of SEQ ID NO: 28. In some embodiments, the polypeptide whose production, structure and/or function is being modulated (or ortholog thereof) comprises a sequence having amino acids 749-799 of SEQ ID NO: 33 and the polypeptide may comprise a sequence that is at least about 35% identical to the sequence of SEQ ID NO: 33. In some embodiments, the polypeptide whose production, structure and/or function is being modulated (or ortholog thereof) comprises a sequence having amino acids 670-720 of SEQ ID NO: 36 and the polypeptide may comprise a sequence that is at least about 35% identical to the sequence of SEQ ID NO: 36. In some embodiments, the polypeptide whose production, structure and/or function is being modulated (or ortholog thereof) comprises a sequence having amino acids 679-729 of SEQ ID NO: 58 and the polypeptide may comprise a sequence that is at least about 35% identical to the sequence of SEQ ID NO: 58. In some embodiments, the polypeptide whose production, structure and/or function is being modulated (or ortholog thereof) comprises a sequence having amino acids 749-799 of SEQ ID NO: 59 and the polypeptide may comprise a sequence that is at least about 35% identical to the sequence of SEQ ID NO: 59.

In some embodiments, variations in sequence may be conservative variations. For example, in some embodiments, when the sequences of the polypeptide whose production, structure and/or function is being modulated (or ortholog thereof) having at least about 35% identity to SEQ ID NO: 1 and comprising a sequence having at least about 95% sequence identity with the amino acids 685-735 of SEQ ID NO: 1 is aligned with the sequence of SEQ ID NO: 1, at least about 50% of the variations between the polypeptide and the sequence of SEQ ID NO: 1 are conservative amino acid substitutions. In some embodiments, at least about 60%, at least about 70%, at least about 80% or at least about 90% of the variations between the polypeptide and the sequence of SEQ ID NO: 1 are conservative amino acid substitutions. In some embodiments, when the sequences of the polypeptide whose production, structure and/or function is being modulated (or ortholog thereof) having at least about 35% identity to SEQ ID NO: 28 and comprising a sequence having at least about 95% sequence identity with the amino acids 753-803 of SEQ ID NO: 28 is aligned with the sequence of SEQ ID NO: 28, at least about 50% of the variations between the polypeptide and the sequence of SEQ ID NO: 28 are conservative amino acid substitutions. In some embodiments, at least about 60%, at least about 70%, at least about 80% or at least about 90% of the variations between the polypeptide and the sequence of SEQ ID NO: 28 are conservative amino acid substitutions. In some embodiments, when the sequences of the polypeptide whose production, structure and/or function is being modulated (or ortholog thereof) having at least about 35% identity to SEQ ID NO: 33 and comprising a sequence having at least about 95% sequence identity with the amino acids 749-799 of SEQ ID NO: 33 is aligned with the sequence of SEQ ID NO: 33, at least about 50% of the variations between the polypeptide and the sequence of SEQ ID NO: 33 are conservative amino acid substitutions. In some embodiments, at least about 60%, at least about 70%, at least about 80% or at least about 90% of the variations between the polypeptide and the sequence of SEQ ID NO: 33 are conservative amino acid substitutions. In some embodiments, when the sequences of the polypeptide whose production, structure and/or function is being modulated (or ortholog thereof) having at least about 35% identity to SEQ ID NO: 36 and comprising a sequence having at least about 95% sequence identity with the amino acids 670-720 of SEQ ID NO: 36 is aligned with the sequence of SEQ ID NO: 36, at least about 50% of the variations between the polypeptide and the sequence of SEQ ID NO: 36 are conservative amino acid substitutions. In some embodiments, at least about 60%, at least about 70%, at least about 80% or at least about 90% of the variations between the polypeptide and the sequence of SEQ ID NO: 36 are conservative amino acid substitutions. In some embodiments, when the sequences of the polypeptide whose production, structure and/or function is being modulated (or ortholog thereof) having at least about 35% identity to SEQ ID NO: 58 and comprising a sequence having at least about 95% sequence identity with the amino acids 679-729 of SEQ ID NO: 58 is aligned with the sequence of SEQ ID NO: 58, at least about 50% of the variations between the polypeptide and the sequence of SEQ ID NO: 58 are conservative amino acid substitutions. In some embodiments, at least about 60%, at least about 70%, at least about 80% or at least about 90% of the variations between the polypeptide and the sequence of SEQ ID NO: 58 are conservative amino acid substitutions. In some embodiments, when the sequences of the polypeptide whose production, structure and/or function is being modulated (or ortholog thereof) having at least about 35% identity to SEQ ID NO: 59 and comprising a sequence having at least about 95% sequence identity with the amino acids 749-799 of SEQ ID NO: 59 is aligned with the sequence of SEQ ID NO: 59, at least about 50% of the variations between the polypeptide and the sequence of SEQ ID NO: 59 are conservative amino acid substitutions. In some embodiments, at least about 60%, at least about 70%, at least about 80% or at least about 90% of the variations between the polypeptide and the sequence of SEQ ID NO: 59 are conservative amino acid substitutions.

In some embodiments, variations in sequence may be conservative variations. For example, in some embodiments, when the sequences of the polypeptide whose production, structure and/or function is being modulated (or ortholog thereof) having at least about 35% identity to SEQ ID NO: 1 and comprising amino acids 685-735 of SEQ ID NO: 1 is aligned with the sequence of SEQ ID NO: 1, at least about 50% of the variations between the polypeptide and the sequence of SEQ ID NO: 1 are conservative amino acid substitutions. In some embodiments, at least about 60%, at least about 70%, at least about 80% or at least about 90% of the variations between the polypeptide and the sequence of SEQ ID NO: 1 are conservative amino acid substitutions. In some embodiments, when the sequences of the polypeptide whose production, structure and/or function is being modulated (or ortholog thereof) having at least about 35% identity to SEQ ID NO: 28 and comprising amino acids 753-803 of SEQ ID NO: 28 is aligned with the sequence of SEQ ID NO: 28, at least about 50% of the variations between the polypeptide and the sequence of SEQ ID NO: 28 are conservative amino acid substitutions. In some embodiments, at least about 60%, at least about 70%, at least about 80% or at least about 90% of the variations between the polypeptide and the sequence of SEQ ID NO: 28 are conservative amino acid substitutions. In some embodiments, when the sequences of the polypeptide whose production, structure and/or function is being modulated (or ortholog thereof) having at least about 35% identity to SEQ ID NO: 33 and comprising amino acids 749-799 of SEQ ID NO: 33 is aligned with the sequence of SEQ ID NO: 33, at least about 50% of the variations between the polypeptide and the sequence of SEQ ID NO: 33 are conservative amino acid substitutions. In some embodiments, at least about 60%, at least about 70%, at least about 80% or at least about 90% of the variations between the polypeptide and the sequence of SEQ ID NO: 33 are conservative amino acid substitutions. In some embodiments, when the sequences of the polypeptide whose production, structure and/or function is being modulated (or ortholog thereof) having at least about 35% identity to SEQ ID NO: 36 and comprising amino acids 670-720 of SEQ ID NO: 36 is aligned with the sequence of SEQ ID NO: 36, at least about 50% of the variations between the polypeptide and the sequence of SEQ ID NO: 36 are conservative amino acid substitutions. In some embodiments, at least about 60%, at least about 70%, at least about 80% or at least about 90% of the variations between the polypeptide and the sequence of SEQ ID NO: 36 are conservative amino acid substitutions. In some embodiments, when the sequences of the polypeptide whose production, structure and/or function is being modulated (or ortholog thereof) having at least about 35% identity to SEQ ID NO: 58 and comprising amino acids 679-729 of SEQ ID NO: 58 is aligned with the sequence of SEQ ID NO: 58, at least about 50% of the variations between the polypeptide and the sequence of SEQ ID NO: 58 are conservative amino acid substitutions. In some embodiments, at least about 60%, at least about 70%, at least about 80% or at least about 90% of the variations between the polypeptide and the sequence of SEQ ID NO: 58 are conservative amino acid substitutions. In some embodiments, when the sequences of the polypeptide whose production, structure and/or function is being modulated (or ortholog thereof) having at least about 35% identity to SEQ ID NO: 59 and comprising amino acids 749-799 of SEQ ID NO: 59 is aligned with the sequence of SEQ ID NO: 59, at least about 50% of the variations between the polypeptide and the sequence of SEQ ID NO: 59 are conservative amino acid substitutions. In some embodiments, at least about 60%, at least about 70%, at least about 80% or at least about 90% of the variations between the polypeptide and the sequence of SEQ ID NO: 59 are conservative amino acid substitutions.

In embodiments in which the % identity threshold is indicated between a polypeptide and an ortholog thereof, the % identity between the full length polypeptides may differ. Two polypeptides may have a % identity as low as 35%, but still be orthologs of each other, provided they additional comprise conserved sequences, for example a sequence having at least about 95% identity to SEQ ID NO: 31 (e.g. a sequence having 100% identity to SEQ ID NO: 31). All of the polypeptides identified in the present invention comprise the sequence of SEQ ID NO: 31. Preferably, the orthologs perform the same function as the reference polypeptide. For example, the orthologs may be a fungal transcription factor that promotes the expression of one or more proteases. Generally, the orthologs are naturally occurring polypeptides.

The orthologs may be from the same genus, for example Trichoderma, Myceliophthora or Aspergillus, or they may be from a different genus. In some cases, different strains of the same species may comprise orthologs. Thus, the ortholog may be from the same species, but a different strain.

In some embodiments, for example when the ortholog is from a different genus, an ortholog may have at least about 35% sequence identity or at least about 40% sequence identity to the full length polypeptide. In some embodiments, the polypeptide may be a polypeptide from a Trichoderma spp. and the ortholog is a polypeptide from a Trichoderma spp. or a Myceliophthora spp. and the ortholog may have at least about 40% sequence identity to the full length polypeptide. In some embodiments, the polypeptide may be a polypeptide from a Myceliophthora spp. and the ortholog is a polypeptide from a Trichoderma spp. or a Myceliophthora spp. and the ortholog may have at least about 40% sequence identity to the full length polypeptide. In some embodiments, for example when the ortholog is from the same genus, an ortholog may have at least about 90% sequence identity to the full length polypeptide. In some embodiments, for example when the ortholog is from the same species, an ortholog may have at least about 95%, at least about 96%, at least about 97%, at least about 98% or at least about 99% sequence identity to the full length polypeptide. In all cases, the orthologs preferably comprise a conserved sequence, such as the sequence of SEQ ID NO: 31 (or a sequence having at least about 95% identity thereto).

In some embodiments, the polypeptide whose production, structure and/or function is being modulated (or ortholog thereof) is a fungal transcription factor that promotes the expression of one or more proteases, and comprises an amino acid sequence having at least about 95% identity to SEQ ID NO: 31, and optionally further comprises a sequence having at least about 35% identity to SEQ ID NO: 1, SEQ ID NO: 28, SEQ ID NO: 33 SEQ ID NO: 36, SEQ ID NO: 58 and/or SEQ ID NO: 59. In some embodiments, the polypeptide whose production, structure and/or function is being modulated (or ortholog thereof) is a fungal transcription factor that promotes the expression of one or more proteases, and comprises the amino acid sequence of SEQ ID NO: 31, and optionally further comprises a sequence having at least about 35% identity to SEQ ID NO: 1, SEQ ID NO: 28, SEQ ID NO: 33, SEQ ID NO: 36 SEQ ID NO: 58 and/or SEQ ID NO: 59.

In some embodiments, the polypeptide whose production, structure and/or function is being modulated (or ortholog thereof) is a fungal transcription factor that promotes the expression of one or more proteases, and comprises an amino acid sequence having at least about 95% identity to SEQ ID NO: 31, and optionally further comprises a sequence having at least about 40% identity to SEQ ID NO: 1, SEQ ID NO: 28, SEQ ID NO: 33, SEQ ID NO: 58 and/or SEQ ID NO: 59. In some embodiments, the polypeptide whose production, structure and/or function is being modulated (or ortholog thereof) is a fungal transcription factor that promotes the expression of one or more proteases, and comprises the amino acid sequence of SEQ ID NO: 31, and optionally further comprises a sequence having at least about 40% identity to SEQ ID NO: 1, SEQ ID NO: 28, SEQ ID NO: 33, SEQ ID NO: 58 and/or SEQ ID NO: 59.

In some embodiments, the polypeptide whose production, structure and/or function is being modulated (or ortholog thereof) is a fungal transcription factor that promotes the expression of one or more proteases, and comprises an amino acid sequence having at least about 95% identity to SEQ ID NO: 31, and optionally further comprises a sequence having at least about 95% identity to SEQ ID NO: 1 and/or SEQ ID NO: 58. In some embodiments, the polypeptide whose production, structure and/or function is being modulated (or ortholog thereof) is a fungal transcription factor that promotes the expression of one or more proteases, and comprises the amino acid sequence of SEQ ID NO: 31, and optionally further comprises a sequence having at least about 95% identity to SEQ ID NO: 1 and/or SEQ ID NO: 58.

In some embodiments, the polypeptide whose production, structure and/or function is being modulated (or ortholog thereof) is a fungal transcription factor that promotes the expression of one or more proteases, and comprises an amino acid sequence having at least about 95% identity to SEQ ID NO: 31, and optionally further comprises a sequence having at least about 90% identity to SEQ ID NO: 28, SEQ ID NO: 33 and/or SEQ ID NO: 59. In some embodiments, the polypeptide whose production, structure and/or function is being modulated (or ortholog thereof) is a fungal transcription factor that promotes the expression of one or more proteases, and comprises the amino acid sequence of SEQ ID NO: 31, and optionally further comprises a sequence having at least about 90% identity SEQ ID NO: 28, SEQ ID NO: 33 and/or SEQ ID NO: 59.

In some embodiments, the polypeptide whose production, structure and/or function is being modulated (or ortholog thereof) is a fungal transcription factor that promotes the expression of one or more proteases, and comprises an amino acid sequence having at least about 95% identity to SEQ ID NO: 31, and optionally further comprises a sequence having at least about 95% identity to SEQ ID NO: 28 and/or SEQ ID NO: 59. In some embodiments, the polypeptide whose production, structure and/or function is being modulated (or ortholog thereof) is a fungal transcription factor that promotes the expression of one or more proteases, and comprises the amino acid sequence of SEQ ID NO: 31, and optionally further comprises a sequence having at least about 95% identity SEQ ID NO: 28 and/or SEQ ID NO: 59.

As used herein a modification, modified or a similar term in the context of polynucleotides refer to modification in a coding or non-coding region of the polynucleotide, such as a regulatory sequence, 5′ untranslated region, 3′ untranslated region, up-regulating genetic element, down-regulating genetic element, enhancer, suppressor, promoter, exon and/or intron region. Modifications may be made to a polynucleotide coding for the polypeptide in the microbial host cell to achieve modification of the at least one polypeptide. As used herein a modification, modified or a similar term in the context of polypeptides, in particular a modification that affects the production, stability and/or function of a polypeptide, may refer to a modification of a polynucleotide coding for the polypeptide. The polynucleotides that are modified in the present invention are polynucleotides that are present in the genome of the parental or wild-type microbial host cell. The modification of these polynucleotides in turn leads to modification of the polypeptides encoded by those polynucleotides.

A modification, modified or a similar term can be a genetic modification, for example a partial or full deletion, that is a partial or full deletion of a gene or polynucleotide encoding the polypeptide. In such deletions, the genomic DNA containing the genetic information for the production of the at least one polypeptide, such as for example a polypeptide having a sequence selected from the group consisting of SEQ ID NOs: 1, 28, 33, 36 58 and 59, of a microbial host cell is removed in its entirety or where at least one nucleotide is removed leading to the modified microbial host cell to produces less of the polypeptide or produces substantially no polypeptide and/or produces a polypeptide having a decreased activity or decreased specific activity or a polypeptide having no activity or no specific activity. The polypeptide is therefore one that is coded for by a gene or polynucleotide in the parental microbial host cell genome. The gene or polynucleotide encoding the polypeptide by may be absent from the genome of the modified microbial host cell (for example in the case of a full deletion) or the gene or polynucleotide may simply be modified to alter its production, stability and/or function.

A modification, modified or a similar term can also be a mutation performed by specific or random mutagenesis, nucleotide insertion and/or nucleotide substitution and/or nucleotide deletion. Such modifications lead to the modified microbial host cell to produce less of the polypeptide or produce substantially no polypeptide and/or produces a polypeptide having a decreased activity or decreased specific activity or a polypeptide having no activity or no specific activity. Modifications may target specific components of the polypeptide. For example, in embodiments where the polypeptide is a regulator of transcription, they may be a modification of one or more of the DNA binding domains of the regulator of transcription. Certain modifications of DNA binding domains of regulators of transcription (for example modification of one or more DNA binding domains by nucleotide deletion, insertion or substitution, but retaining other DNA binding domains as unmodified) may allow the selective downregulation of proteases, but may have no effect on the expression of other proteins under the control of the regulator of transcription, such as cellulases.

In some embodiments, the regulator of transcription is a promoter of transcription that has been modified to decrease the function of one or more DNA binding domains in the promoter of transcription that positively control (directly or indirectly) the expression of one or more proteases. Decreasing the function of one or more DNA binding domains in the promoter of transcription that cause, initiate or promote the expression of one or more proteases may comprise decreasing the affinity of one or more DNA binding domains of the promoter of transcription that bind to one or more promoters that cause, initiate or promote the expression of one or more protease genes (direct control), or may comprise decreasing the affinity of one or more DNA binding domains of the promoter of transcription that bind to one or more promoters that cause, initiate or promote the expression of one or more genes that cause, initiate or promote the expression of one or more protease genes (indirect control). In some embodiments, any DNA binding domains in the promoter of transcription that cause, initiate or promote the expression of one or more cellulase genes have not been modified (that is, any DNA binding domains in the promoter of transcription that bind to promoters that cause, initiate or promote the expression of one or more cellulase genes are not modified). Alternatively, one or more DNA binding domains in the promoter of transcription that cause, initiate or promote the expression of one or more cellulase genes may have been modified to increase their affinity for the promoters that cause, initiate or promote the expression of the one or more cellulase genes.

The above selective modification of DNA binding domains may also be exploited in embodiments in which the activity of the repressors of transcription are modulated. For example, in some embodiments, a repressor of transcription may been modified to increase the function of one or more DNA binding domains in the repressor of transcription that adversely control (directly or indirectly) the expression of one or more proteases. Increasing the function of one or more DNA binding domains in the repressor of transcription that adversely affects the expression of one or more proteases may comprise increasing the affinity of one or more DNA binding domains of the repressor of transcription that bind to DNA in such a way to inhibit the expression of one or more protease genes (direct control), or may comprise increasing the affinity of one or more DNA binding domains of the repressor of transcription that bind to DNA in such a way as to inhibit the expression of one or more genes that cause, initiate or promote the expression of one or more protease genes (indirect control).

Modifications of the DNA binding domains of regulators of transcription (promoters of transcription or repressors of transcription) might not change the expression of the regulator of transcription by the microbial host cell. For example, the level of expression of the regulator of transcription may be the same in the modified microbial host cell compared to a parental or wild-type microbial host cell (when measured under the same or substantially the same conditions). However, given the selective modification of the DNA binding domains of the regulator of transcription, the activity of those regulators of transcription may still be modulated to affect the desired decrease in activity of one or more proteases expressed by the microbial host cell.

A modification, modified or a similar term can also involve targeting the polypeptide, its corresponding chromosomal gene and/or its corresponding mRNA by techniques well known in the art such as anti-sense techniques, RNAi techniques, CRISPR techniques, ADAR techniques, Zinc-finger nuclease (ZFN) techniques, transcription activator-like effector nuclease (TALEN) techniques, a small molecule inhibitor, antibody, antibody fragment or a combination thereof leading to the modified microbial host cell to produces less of the polypeptide or produces substantially no polypeptide and/or produces a polypeptide having a decreased activity or decreased specific activity or a polypeptide having no activity or no specific activity and/or where an interaction with the polypeptide by specific or non-specific binding leads to degradation, precipitation of the polypeptide, or where this interaction leads to the polypeptide having decreased activity or decreased specific activity or a having no activity or no specific activity. As noted above, modifications may be selective modification that target particular DNA binding domains when the polypeptide is a regulator of transcription (for example a promoter of transcription or a repressor of transcription).

A modification, modified or similar term in the context of increasing the production, stability and/or function of at least one polypeptide may be a modification that increases the expression and/or activity and/or stability of the at least one polypeptide. Such modifications may be, for example, overexpression of the polypeptide in the microbial host cell (for example insertion of additional copies of polynucleotides coding for the polypeptide either directly into the chromosome of the modified microbial host cell or by expression of the at least one polypeptide or in the form of an episomal DNA under control of a constitutive or inducible promoter), a mutation in the polypeptide that increases is activity, or contacting the microbial host cell with a compound that increases the activity and/or expression of the polypeptide. As noted above, modifications may be selective modification that target particular DNA binding domains when the polypeptide is a promoter of transcription or repressor of transcription.

A microbial host cell, be it a microbial host cell which has been modified or a parent host cell, is defined here as a single cellular organism used during a fermentation process or during cell culture to produce a compound of interest. Preferably, a microbial host cell is selected from the kingdom Fungi. In particular, the fungus may be a filamentous fungus.

The fungi may preferably be from the division Ascomycota, subdivision Pezizomycotina. In some embodiments, the fungi may preferably from the Class Sordariomycetes, optionally the Subclass Hypocreomycetidae. In some embodiments, the fungi may be from an Order selected from the group consisting of Hypocreales, Microascales, Eurotiales, Onygenales and Sordariales. In some embodiments, the fungi may be from a Family selected from the group consisting of Hypocreaceae, Nectriaceae, Clavicipitaceae and Microascaceae. In some more specific embodiments, the fungus may be from a Genus selected from the group consisting of Trichoderma (anamorph of Hypocrea), Myceliophthora, Fusarium, Gibberella, Nectria, Stachybotrys, Claviceps, Metarhizium, Villosiclava, Ophiocordyceps, Cephalosporium, Neurospora, Rasamsonia and Scedosporium. In some further and more specific embodiments, the fungi may be selected from the group consisting of Trichoderma reesei (Hypocrea jecorina), T. citrinoviridae, T. longibrachiatum, T. virens, T. harzianum, T. asperellum, T. atroviridae, T. parareesei, Fusarium oxysporum, F. gramineanum, F. pseudograminearum, F. venenatum, Gibberella fujikuroi, G. moniliformis, G. zeaea, Nectria (Haematonectria) haematococca, Stachybotrys chartarum, S. chlorohalonata, Claviceps purpurea, Metarhizium acridum, M. anisopliae, Villosiclava virens, Ophiocordyceps sinensis, Neurospora crassa, Rasamsonia emersoniim, Acremonium (Cephalosporium) chrysogenum, Scedosporium apiospermum, Aspergillus niger, A. awamori, A. oryzae, A. nidulans, Chrysosporium lucknowense,

Thermothelomyces thermophilus, Myceliophthora thermophila, Myceliophthora heterothallica, Thermothelomyces heterothallica, Humicola insolens, and Humicola grisea, most preferably Trichoderma reesei or Myceliophthora heterothallica. If the host cell is a Trichoderma reesei cell, it may be selected from the following group of Trichoderma reesei strains obtainable from public collections: QM6a, ATCC13631; RutC-30, ATCC56765; QM9414, ATCC26921, RL-P37 and derivatives thereof. If the host cell is a Myceliophthora heterothallica, it may be selected from the following group of Myceliophthora heterothallica or Thermothelomyces thermophilus strains: CBS 131.65, CBS 203.75, CBS 202.75, CBS 375.69, CBS 663.74 and derivatives thereof. If the host cell is a Myceliophthora thermophila it may be selected from the following group of Myceliophthora thermophila strains ATCC42464, ATCC26915, ATCC48104, ATCC34628, Thermothelomyces heterothallica C1, Thermothelomyces thermophilus M77 and derivatives thereof. If the host cell is an Aspergillus nidulans it may be selected from the following group of Aspergillus nidulans strains: FGSC A4 (Glasgow wild-type), GR5 (FGSC A773), TN02A3 (FGSC A1149), TNO2A25, (FGSC A1147), ATCC 38163, ATCC 10074 and derivatives thereof.

Proteases are herein defined as enzymes that catalyze a proteolysis reaction which is the breakdown of proteins into smaller fragments or into individual amino acids and where the proteolysis reaction occurs either at specific recognition sites or at random sites. These proteases include, but are not limited to, serine protease, cysteine proteases, threonine proteases aspartic proteases glutamic proteases metalloproteases and asparagine peptide lyases.

The modulation in protease activity is the modulation of activity of at least one protease expressed by the microbial host cell, typically at least one protease encoded by the genome of the microbial host cell. In some embodiments, the microbial host cell may have modulation of protease activity (for example a reduction or deficiency in protease activity) for a range of different proteases. The identity of the proteases whose activity is modulated will depend on the one or more polypeptides whose production, stability and/or function is being modified, since different polypeptides (such as regulators of transcription) will control the expression of different proteases. In other embodiments, the microbial host cell may have modulation of protease activity for one or more specific proteases, in addition to the modification to the one or more polypeptides (which are regulators of transcription that directly or indirectly control the expression of one or more proteases).

With a modulation in protease activity, it is generally meant the protease activity of the modified microbial host cell is reduced or is deficient, compared to a parent microbial host cell (which has not been modified to modulate its protease activity). In preferred embodiments, in particular embodiments relating to the production of recombination proteins expressed by the microbial host cell, the modulation of protease activity is a decrease in protease activity.

The protease activity of the microbial host cell is the overall protease activity of the microbial host cell. This refers to ability of the microbial host cell to breakdown proteins as a result of secretion of proteases into the extracellular environment. In some embodiments, this may be achieved by the modulation (e.g. decrease) of activity of one protease. However, advantageously, in particular in embodiments in which the polypeptide that is modified is a promoter of transcription or repressor of transcription, the modulation (e.g. decrease) of protease activity is the modulation (e.g. decrease) or a plurality of proteases. In such embodiments, a single modification may have an overall modulation (e.g. reduction) in protease activity that is great than when the modification is of the protease itself, since the polypeptide may control the expression of a plurality of proteases. The present invention therefore provides advantages over embodiments in which modifications must be made to proteases individually, since a single modification can modulate the expression of a plurality of proteases expressed by the microbial host cell.

With a reduction or deficiency in protease activity it is meant that in the cell culture media or fermentation broth and/or the intracellular environment of a microbial host cell, proteases are reduced in activity, abundance either in total numbers and/or in number of different kinds of proteases when compared with a parent microbial host cell and measured under the same or substantially the same conditions. So, when the microbial cells are cultured in a culture medium or fermentation broth, the overall protease activity is reduced. This reduction or deficiency may be at least about 1% less protease activity if compared with the parent host cell and measured under the same or substantially the same conditions, at least about 5% less, at least about 10% less, at least about 20% less, at least about 30% less, at least about 40% less, at least about 50% less, at least about 60% less, at least about 70% less, at least about 80% less, at least about 90% less, at least about 91% less, at least about 92% less, at least about 93% less, at least about 94% less at least about 95% less, at least about 96% less, at least about 97% less, at least about 98% less, at least about 99% less, or at least about 99.9% less, for example the microbial host cell has substantially no protease activity if compared with the parent host cell and measured under the same or substantially the same conditions. In preferred embodiments, the modified microbial host cell may have at least about a 40% reduction in protease activity compared to a microbial host cell that has not been modified (i.e. a parental microbial host cell).

In one embodiment the culture media or fermentation broth containing the microbial host cell that has been modified contains a protease activity which is reduced by at least about 1% if compared with the culture media or fermentation broth of the parent host cell and measured under the same or substantially the same conditions, for example at least about 5% less, at least about 10% less, at least about 20% less, at least about 30% less, at least about 40% less, at least about 50% less, at least about 60% less, at least about 70% less, at least about 80% less, at least about 90% less, at least about 91% less, at least about 92% less, at least about 93% less, at least about 94% less at least about 95% less, at least about 96% less, at least about 97% less, at least about 98% less, at least about 99% less, or at least about 99.9% less, or the culture media or fermentation broth contains substantially no protease activity if compared with the parent host cell and measured under the same or substantially the same conditions. In preferred embodiments the culture media or fermentation broth containing microbial host cells that have been modified has a protease activity which is reduced by at least 40% compared to culture media or fermentation broth containing microbial host cells that have not been modified (i.e. a parental microbial host cell). “Containing” in this context refers to the culture media or fermentation broth that has been used to culture or ferment either the modified microbial host cell, or a parental microbial host cell.

In another embodiment the intracellular environment of the microbial host that has been modified contains a protease activity which is reduced by at least about 1% if compared with the culture media or fermentation broth of the parent host cell and measured under the same or substantially the same conditions, for example at least about 5% less, at least about 10% less, at least about 20% less, at least about 30% less, at least about 40% less, at least about 50% less, at least about 60% less, at least about 70% less, at least about 80% less, at least about 90% less, at least about 91% less, at least about 92% less, at least about 93% less, at least about 94% less at least about 95% less, at least about 96% less, at least about 97% less, at least about 98% less, at least about 99% less, or at least about 99.9% less, or the culture media or fermentation broth contains no substantially no protease activity if compared with the parent host cell and measured under the same or substantially the same conditions. In preferred embodiments the intracellular environment of the microbial host that has been modified contains a protease activity which is reduced by at least 40% compared to a microbial host cell that has not been modified (i.e. a parental microbial host cell).

A reduction in intracellular protease activity may be a result of a reduction in the production, stability and/or function of the protease. A reduction in extracellular protease activity (i.e. activity of protease in the culture media or fermentation broth) may be a result of a reduction in the production, stability and/or function of the protease. Alternatively or additionally, the reduction may be a result of a reduction in the secretion of the protease into the culture media or fermentation broth. Accordingly, in some embodiments, the modified microbial host cell may have a reduction in the secretion of one or more proteases compared with a parent microbial host cell which has not been modified.

In embodiments in which the microbial host cell comprises a nucleotide sequence coding for a compound of interest, the reduction in protease activity (for example at least about a 40% reduction) may be present during conditions suitable for or conducive to the production of the compound of interest by the microbial host cell. In this way, the microbial host cell can produce higher yields of the compound of interest.

Advantageously, although not essentially, in some embodiments the modified microbial host cell may have substantially no decrease in cellulase (for example cellobiohydrolase) activity compared to a parental microbial host cell. In particular, in some embodiments, the modified microbial host cell may have substantially no decrease in cellobiohydrolase activity compared to a parental microbial host cell. “Substantially no decrease” means there may be a decrease of up to about 20%, up to about 10% or up to about 5% in cellulase (for example cellobiohydrolase) activity in the modified microbial host cell compared to a parental microbial host cell that has not had a modification to affect the production, stability and/or function of the at least one polypeptide. In some embodiments, avoiding a decrease in cellulase production may be beneficial. For example, if cellulase production is not disrupted, this may indicate promoters of cellulase productions are not disrupted. Such promoters are useful when the microbial host cells are used to express a compound of interest, since the compound of interest may be present in the form of an expression construct with the sequence encoding the compound of interest being under the control of a promoter. Useful promoters include cellulase promoters since genes under the control of such promoters are constitutively expressed and expressed in a high yield. Therefore, retaining the ability to use cellulase promoters for the expression of compounds of interest, such as chb1, may be beneficial.

With a “compound of interest” it is meant any recombinant protein such as an antibody or a functional fragment thereof, a carbohydrate-binding domain, a heavy chain antibody or a functional fragment thereof, a single domain antibody, a heavy chain variable domain of an antibody or a functional fragment thereof, a heavy chain variable domain of a heavy chain antibody or a functional fragment thereof, a variable domain of camelid heavy chain antibody (VHH) or a functional fragment thereof, a variable domain of a new antigen receptor a variable domain of shark new antigen receptor (vNAR) or a functional fragment thereof, a minibody, a nanobody, a nanoantibody, an affibody, an alphabody, a designed ankyrin-repeat domain, an anticalins, a knottins or an engineered CH2 domain. In some embodiments, the compound of interest is an antibody, for example a VHH.

In some embodiments, the compound of interest is a therapeutic protein, biosimilar, multi-domain protein, peptide hormone, antimicrobial peptide, peptide, carbohydrate-binding module, enzyme, cellulase, protease, protease inhibitor, aminopeptidase, amylase, carbohydrase, carboxypeptidase, catalase, chitinase, cutinase, deoxyribonuclease, esterase, alpha-galactosidase, beta-galactosidase, glucoamylase, alpha-glucosidase, beta-glucosidase, invertase, laccase, lipase, mannanase, mutanase, oxidase, pectinolytic enzyme, peroxidase, phospholipase, phytase, phosphatase, polyphenoloxidase, redox enzyme, proteolytic enzyme, ribonuclease, transglutaminase or xylanase.

In some embodiments, the compound of interest is a VHH. In more specific embodiments, the VHH may be a VHH bind a specific lipid fraction of the cell membrane of a fungal spore. Such VHHs may exhibit fungicidal activity through retardation of growth and/or lysis and explosion of spores, thus preventing mycelium formation. The VHH may therefore have fungicidal or fungistatic activity.

In some embodiments, the VHH may be a VHH that is capable of binding to a lipid-containing fraction of the plasma membrane of a fungus (for example Botrytis cinerea or other fungus). Said lipid-containing fraction may be obtainable by chromatography. For example, said lipid-containing fraction may be obtainable by a method comprising:

fractionating hyphae of a fungus (for example Botrytis cinerea or other fungus) by total lipid extract thin-layer chromatography and selecting the fraction with a Retention Factor (Rf) higher than the ceramide fraction and lower than the non-polar phospholipids fraction.

The invention also provides a polypeptide, wherein said at least one polypeptide is capable of binding to a lipid-containing fraction of the plasma membrane of a fungus (for example Botrytis cinerea or other fungus). Said lipid-containing fraction may be obtainable by chromatography. For example, said lipid-containing fraction may be obtainable by a method comprising:

fractionating hyphae of a fungus (for example Botrytis cinerea or other fungus) by total lipid extract thin-layer chromatography and selecting the fraction with a Retention Factor (Rf) higher than the ceramide fraction and lower than the non-polar phospholipids fraction.

The VHHs are generally capable of binding to a fungus. The VHH thereby causes retardation of growth of a spore of the said fungus and/or lysis of a spore of the said fungus. That is to say, binding of the VHH to a fungus results in retardation of growth of a spore of the said fungus and/or lysis of a spore of the said fungus.

The VHHs may (specifically) bind to a membrane of a fungus or a component of a membrane of a fugus. In some embodiments, the VHHs do not (specifically) bind to a cell wall or a component of a cell wall of a fungus. For example, in some embodiments, the VHHs do not (specifically) bind to a glucosylceramide of a fungus.

The VHHs may be capable of (specifically) binding to a lipid-containing fraction of the plasma membrane of a fungus, such as for example a lipid-containing fraction of Botrytis cinerea or other fungus. Said lipid-containing fraction (of Botrytis cinerea or otherwise) may be obtainable by chromatography. The chromatography may be performed on a crude lipid extract (also referred to herein as a total lipid extract, or TLE) obtained from fungal hyphae and/or conidia. The chromatography may be, for example, thin-layer chromatography or normal-phase flash chromatography. The chromatography (for example thin-layer chromatography) may be performed on a substrate, for example a glass plate coated with silica gel. The chromatography may be performed using a chloroform/methanol mixture (for example 85/15% v/v) as the eluent.

For example, said lipid-containing fraction may be obtainable by a method comprising:

fractionating hyphae and/or conidia of a fungus (for example Botrytis cinerea or other fungus) by total lipid extract thin-layer chromatography and selecting the fraction with a Retention Factor (Rf) higher than the ceramide fraction and lower than the non-polar phospholipids fraction.

In a more specific embodiment, the lipid-containing fraction may be obtainable by a method comprising:

fractionating hyphae and/or conidia of a fungus (for example Botrytis cinerea or other fungus) by total lipid extract thin-layer chromatography on a silica-coated glass slide using a chloroform/methanol mixture (for example 85/15% v/v) as the eluent and selecting the fraction with a Retention Factor (Rf) higher than the ceramide fraction and lower than the non-polar phospholipids fraction.

Alternatively, the fraction may be obtained using normal-phase flash chromatography. In such a method, the method may comprise:

fractionating hyphae and/or conidia of a fungus (for example Botrytis cinerea or other fungus) by total lipid extract normal-phase flash chromatography, and selecting the fraction with a Retention Factor (Rf) higher than the ceramide fraction and lower than the non-polar phospholipids fraction.

In a more specific embodiment, the lipid-containing fraction may be obtainable by a method comprising:

fractionating hyphae and/or conidia of a fungus (for example Botrytis cinerea or other fungus) by total lipid extract normal-phase flash chromatography comprising dissolving the TLE in dichloromethane (CH₂Cl₂) and MeOH and using CH₂Cl₂/MeOH (for example 85/15%, v/v) as the eluent, followed by filtration of the fractions through a filter.

In a more specific embodiment, the lipid-containing fraction may be obtainable by a method comprising:

fractionating hyphae and/or conidia of a fungus (for example Botrytis cinerea or other fungus) by total lipid extract normal-phase flash chromatography comprising dissolving the TLE in dichloromethane (CH₂Cl₂) and MeOH loading the TLE on to a phase flash cartridge (for example a flash cartridge with 15 μm particles), running the column with CH₂Cl₂/MeOH (85/15%, v/v) as the eluent, and filtering the fractions through a filter (for example a 0.45 μm syringe filter with a nylon membrane) and drying the fractions.

The fractions from the chromatography may be processed prior to testing of binding of the VHH to the fraction or of interaction with the fraction. For example, liposomes comprising the fractions may be prepared. Such a method may comprise the use of thin-film hydration. For example, in such a method, liposomes may be prepared using thin-film hydration with the addition of 1,6-diphenyl-1,3,5-hexatriene (DPH). Binding and/or disruption of the membranes by binding of the VHH may be measured by a change in fluorescence before and after polypeptide binding (or by reference to a suitable control).

Accordingly, in some embodiments, the VHHs may (specifically) bind to a lipid-containing chromatographic fraction of the plasma membrane of a fungus, optionally wherein the lipid-containing chromatographic fraction is prepared into liposomes prior to testing the binding of the polypeptide thereto.

Binding of the VHH to a lipid-containing fraction of a fungus may be confirmed by any suitable method, for example bio-layer interferometry. Specific interactions with the lipid-containing fractions may be tested. For example, it may be determined if the polypeptide is able to disrupt the lipid fraction when the fraction is prepared into liposomes, for example using thin-film hydration.

In methods involving chromatography, an extraction step may be performed prior to the step of chromatography. For example, fungal hyphae and/or conidia may be subjected to an extraction step to provide a crude lipid extract or total lipid extract on which the chromatography is performed. For example, in some embodiments, fungal hyphae and/or conidia (for example fungal hyphae and/or conidia of Fusarium oxysporum or Botrytis cinerea) may be extracted at room temperature, for example using chloroform:methanol at 2:1 and 1:2 (v/V) ratios. Extracts so prepared may be combined and dried to provide a crude lipid extract or TLE.

Accordingly, in some embodiments, the VHH may be capable of (specifically) binding to a lipid-containing fraction of the plasma membrane of a fungus (such as Fusarium oxysporum or Botrytis cinerea), wherein the lipid-containing fraction of the plasma membrane of the fungus is obtained or obtainable by chromatography. The chromatography may be normal-phase flash chromatography or thin-layer chromatography. Binding of the VHH to the lipid to the lipid-containing fraction may be determined according to bio-layer interferometry. In some embodiments, the chromatography step may be performed on a crude lipid fraction obtained or obtainable by a method comprising extracting lipids from fungal hyphae and/or conidia from a fungal sample. The extraction step may use chloroform:methanol at 2:1 and 1:2 (v/v) ratios to provide two extracts, and then combining the extracts.

In methods relating to thin-layer chromatography, the chromatography may comprise the steps of:

fractionating hyphae of the fungus by total lipid extract thin-layer chromatography and selecting the fraction with a Retention Factor (Rf) higher than the ceramide fraction and lower than the non-polar phospholipids fraction.

In some methods relating to thin-layer chromatography, the chromatography may comprise the steps of:

fractionating hyphae and/or conidia of a fungus (for example Botrytis cinerea or other fungus) by total lipid extract thin-layer chromatography on a silica-coated glass slide using a chloroform/methanol mixture (for example 85/15% v/v) as the eluent and selecting the fraction with a Retention Factor (Rf) higher than the ceramide fraction and lower than the non-polar phospholipids fraction.

In methods relating to normal-phase flash chromatography, the chromatography may comprise the steps of:

fractionating hyphae and/or conidia of a fungus (for example Botrytis cinerea or other fungus) by total lipid extract normal-phase flash chromatography, and selecting the fraction with a Retention Factor (Rf) higher than the ceramide fraction and lower than the non-polar phospholipids fraction.

In some methods relating to normal-phase flash chromatography, the chromatography may comprise the steps of:

fractionating hyphae and/or conidia of a fungus (for example Botrytis cinerea or other fungus) by total lipid extract normal-phase flash chromatography comprising dissolving the TLE in dichloromethane (CH₂Cl₂) and MeOH and using CH₂Cl₂/MeOH (for example 85/15%, v/v) as the eluent, followed by filtration of the fractions through a filter.

In some methods relating to normal-phase flash chromatography, the chromatography may comprise the steps of:

fractionating hyphae and/or conidia of a fungus (for example Botrytis cinerea or other fungus) by total lipid extract normal-phase flash chromatography comprising dissolving the TLE in dichloromethane (CH₂Cl₂) and MeOH loading the TLE on to a phase flash cartridge (for example a flash cartridge with 15 μm particles), running the column with CH₂Cl₂/MeOH (85/15%, v/v) as the eluent, and filtering the fractions through a filter (for example a 0.45 μm syringe filter with a nylon membrane) and drying the fractions.

In some embodiments, the compound of interest is VHH-1, VHH-2 or VHH-3. For example, in some embodiments, the compound of interest is a VHH comprising or consisting of a sequence selected from the group consisting of SEQ ID NOs: 43, 44, 48, 52, 56 and 57.

In some embodiments, the compound of interest is a VHH comprising:
(a) a CDR1 comprising or consisting of a sequence selected from the group consisting of SEQ ID NOs 45, 49 and 53;
(b) a CDR2 comprising or consisting of a sequence selected from the group consisting of SEQ ID NOs: 46, 50 and 54; and
(c) a CDR3 comprising or consisting of a sequence selected from the group consisting of SEQ ID NOs: 47, 51 and 55.

In some embodiments, the compound of interest is a VHH comprising:
(a) a CDR1 comprising or consisting of the sequence of SEQ ID NO: 45, a CDR2 comprising or consisting of the sequence of SEQ ID NO: 46 and a CDR3 comprising or consisting of the sequence of SEQ ID NO: 47;
(b) a CDR1 comprising or consisting of the sequence of SEQ ID NO: 49, a CDR2 comprising or consisting of the sequence of SEQ ID NO: 50 and a CDR3 comprising or consisting of the sequence of SEQ ID NO: 51 or
(c) a CDR1 comprising or consisting of the sequence of SEQ ID NO: 53, a CDR2 comprising or consisting of the sequence of SEQ ID NO: 54 and a CDR3 comprising or consisting of the sequence of SEQ ID NO: 55.

In some embodiments, the compound of interest is a VHH comprising a CDR1 comprising or consisting of the sequence of SEQ ID NO: 45, a CDR2 comprising or consisting of the sequence of SEQ ID NO: 46 and a CDR3 comprising or consisting of the sequence of SEQ ID NO: 47.

In some embodiments, the compound of interest is a VHH comprising SEQ ID NO: 43.

In some embodiments, the compound of interest is a VHH comprising SEQ ID NO: 44.

In some embodiments, the compound is a VHH disclosed in WO2014/177595 or WO2014/191146, the entire contents of which are incorporated herein by reference.

Thus the microbial host cells of the invention can be used to produce compounds of interest, in particular VHHs, such as the VHHs disclosed herein, as well as other VHHs, such as those disclosed in WO2014/177595 or WO2014/191146. In some embodiments, the VHHs are fused to a carrier peptide.

With “capable of expressing a compound of interest” it is meant that the microbial host cell is modified in such a way that it contains the genetic information of a compound of interest that is under control of a promoter sequence that drives the expression of said compound either in a continuous manner or during conditions suitable for expression. For example, in some embodiments, the microbial host cell may comprise a polynucleotide coding for the compound of interest. The polynucleotide may be in the form of a plasmid or a vector. The polynucleotide may be introduced into the microbial host cell according to any suitable method known to the skilled person. For example, the polynucleotide may be introduced into the cell by transformation, for example protoplast-mediated transformation (PMT), Agrobacterium-mediated transformation (AMT), electroporation, biolistic transformation (particle bombardment), or shock-wave-mediated transformation (SWMT). The compound of interest is therefore a recombinant or heterologous compound of interest, since it is not encoded by the wild-type genome of the microbial host cell.

The compound of interest may be under the control of (i.e. may be operably linked to) a promoter sequence. The promoter sequence may promote the expression of the compound of interest in and by the modified microbial host cell. In some embodiments, the compound of interest may be operably linked to a constitutive promoter, or the compound of interest may be operably linked to an inducible promoter. When linked to a inducible promoter, methods of the invention may comprise a step of inducing expression of the compound of interest by the microbial host cell.

With a “promoter sequence” it is meant a nucleotide sequence that is preferably recognized by a polypeptide, for example a regulator of transcription or at the very least allows the correct formation of a RNA-polymerase complex in such a way that expression of a compound of interest, of which the polynucleotide is located downstream of the promoter sequence as is well known in the art, is established in a continuous manner or during conditions suitable for expression, as to produce the compound of interest or a compound involved in the production of the compound of interest. The promoters are generally promoters that are functional in fungi. These promoters can be but are not limited to alcA Alcohol dehydrogenase I, amyB TAKA-amylase A, bli-3 Blue light-inducible gene, bphA Benzoate p-hydrolase, catR Catalase, cbh1 (cbhl) Cellobiohydrolase I, cbh2 (cbhll) cellobiohydrolase 2, cel5a endoglucanase 2, cel12a endogluconase 3, cre1 Glucose repressor, exylA endoxylanase, gas 1,3-beta-glucanosyltransferase, glaA Glucoamylase A, gla1 Glucoamylase, mir1 Siderophore transporter, niiA Nitrite reductase, qa-2 Catabolic 3-dehydroquinase, Smxyl endoxylanase, tcu-1 Copper transporter, thiA thiamine thiazole synthase, vvd Blue light receptor, xyl1 endoxylanase, xyIP endoxylanase, xyn1 endoxylanase 1, xyn2 endoxylanase 2, xyn3 endoxylanase 3, zeaR regulator of transcription, cDNA1, eno1 enolase, gpd1 glyceraldehyde-3-phosphate dehydrogenase, pdc1 pyruvate decarboxylase, pki1 pyruvate kinase, tef1 transcription elongation factor 1a, rp2 ribosomal protein, stp1 sugar transporter or tauD3 tauD like dioxygenase.

As used herein, the terms “polypeptide”, “protein”, “peptide”, and “amino acid sequence” are used interchangeably, and refer to a polymeric form of amino acids of any length, which can include coded and non-coded amino acids, chemically or biochemically modified or derivatized amino acids, and polypeptides having modified peptide backbones.

In some embodiments the compound of interest is a polypeptide that is fused to a second polypeptide and where the second polypeptide is a “carrier peptide”. In other words, the microbial host cell may comprise a polynucleotide sequence encoding a polypeptide fused to a carrier peptide. Carrier peptides are peptides that may be produced and secreted by the microbial host cell. Carrier peptides may be abundant or produced in quantities that exceed other peptides not suitable to be used as a carrier peptide. Carrier peptides may be native to the microbial host cell. Thus, carrier peptides may serve to increase the production and/or the secretion of the compound of interest as compared to the production and/or secretion of a compound of interest not fused to a carrier peptide. Carrier peptides may be, but are not limited to, a glucoamylase Gla peptide, a cellobiohydrolase Cbh1 peptide or a cellobiohydrolase cbh2 peptide. Carrier peptides may consist of a functional fragment of, but not limited to, glucoamylase Gla peptide, a cellobiohydrolase Cbh1 peptide or a cellobiohydrolase cbh2 peptide. A functional fragment of a carrier peptide may be limited to the N-terminal region of, but not limited to, glucoamylase Gla peptide, a cellobiohydrolase Cbh1 peptide or a cellobiohydrolase cbh2 peptide. Alternatively the functional fragment of a carrier peptide may be limited to the catalytic domain of the carrier peptide, such as the catalytic domain of the cbh1 carrier peptide. The N-terminal region may consist of only the signal peptide or signal sequence of, but not limited to glucoamylase Gla peptide, a cellobiohydrolase Cbh1 peptide or a cellobiohydrolase cbh2 peptide. The signal peptide or signal sequence may allow for the secretion of the compound of interest. In more preferred embodiments the carrier peptide is fused to the N-terminus of the compound of interest. In some embodiments the compound of interest and the carrier peptide may be separated by a proteolytic cleavage site. That is to say, a third peptide containing a proteolytic cleavage site can be present between the compound of interest and the carrier peptide. In a more preferred embodiment the proteolytic cleavage site is fused to the C-terminus of the carrier-peptide and the N-terminus of the compound of interest. Thus, the polypeptide may be a fusion protein comprising, in a 5′ to 3′ order, a carrier peptide, a proteolytic cleavage site, and the compound of interest. The proteolytic cleavage site may be, but is not limited to, the KexB proteolytic cleavage site. The presence of a proteolytic cleavage site allows for the compound of interest to be separated from the carrier peptide by action of a protease. This protease may be but is not limited to the KexB protease. In some embodiments this separation takes place at the time of secretion or immediately after secretion of the fusion protein. In other embodiments the protease separating the compound of interest can be added to the fermentation medium. In some embodiments the protease separating the compound of interest can be added during or after purification of the fusion protein. In a preferred embodiment the separation of the compound of interest from the carrier peptide can occur by protease activity native to the microbial host cell.

As used herein, the terms “nucleic acid molecule”, “polynucleotide”, “polynucleic acid”, “nucleic acid” are used interchangeably and refer to a polymeric form of nucleotides of any length, either deoxyribonucleotides or ribonucleotides, or analogs thereof. Polynucleotides may have any three-dimensional structure, and may perform any function, known or unknown. Non-limiting examples of polynucleotides include a gene, a gene fragment, exons, introns, messenger RNA (mRNA), transfer RNA, ribosomal RNA, ribozymes, cDNA, recombinant polynucleotides, branched polynucleotides, plasmids, vectors, isolated DNA of any sequence, control regions, isolated RNA of any sequence, nucleic acid probes, and primers. The nucleic acid molecule may be linear or circular.

As used herein, the term “homology” denotes at least secondary structural similarity between two macromolecules, particularly between two polypeptides or polynucleotides, from same or different taxons, wherein said similarity is due to shared ancestry. Hence, the term “homologues” denotes so-related macromolecules having said secondary and optionally tertiary structural similarity.

For comparing two or more nucleotide sequences, sequence “identity” may be used, in which the “(percentage of) sequence identity” between a first nucleotide sequence and a second nucleotide sequence may be calculated using methods known by the person skilled in the art.

The terms “sequence identity”, “% identity” are used interchangeable herein. For the purposes of this invention, it is defined here that in order to determine the percentage of sequence identity of two amino acid sequences or of two nucleic acid sequences, the sequences are aligned for optimal comparison purposes. In order to optimize the alignment between the two sequences gaps may be introduced in any of the two sequences that are compared. Such alignment can be carried out over the full length of the sequences being compared.

Alternatively, the alignment may be carried out over a shorter length, for example over about 20, about 50, about 100 or more nucleic acids/bases or amino acids. The sequence identity is the percentage of identical matches between the two sequences over the reported aligned region. The percent sequence identity between two amino acid sequences or between two nucleotide sequences may be determined using the Needleman and Wunsch algorithm for the alignment of two sequences (Needleman, S. B. and Wunsch, C. D. (1970) J. Mol. Biol. 48, 443-453). Both amino acid sequences and nucleotide sequences can be aligned by the algorithm. The Needleman-Wunsch algorithm has been implemented in the computer program NEEDLE. For the purpose of this invention the NEEDLE program from the EMBOSS package may be used (version 2.8.0 or higher, EMBOSS: The European Molecular Biology Open Software Suite (2000) Rice, Longden and Bleasby, Trends in Genetics 16, (6) pp276-277, http://emboss.bioinformatics.nl/).

For protein sequences EBLOSUM62 is used for the substitution matrix. For nucleotide sequence, EDNAFULL is used. The optional parameters used are a gap-open penalty of 10 and a gap extension penalty of 0.5. The skilled person will appreciate that all these different parameters will yield slightly different results but that the overall percentage identity of two sequences is not significantly altered when using different algorithms.

After alignment by the program NEEDLE as described above the percentage of sequence identity between a query sequence and a sequence of the invention is calculated as follows: number of corresponding positions in the alignment showing an identical amino acid or identical nucleotide in both sequences divided by the total length of the alignment after subtraction of the total number of gaps in the alignment. The identity as defined herein can be obtained from NEEDLE by using the NOBRIEF option and is labeled in the output of the program as “longest identity”. If both amino acid sequences which are compared do not differ in any of their amino acids over their entire length, they are identical or have 100% identity. Amino acid sequences and nucleic acid sequences are said to be “exactly the same” or “identical” if they have 100% sequence identity over their entire length.

In determining the degree of sequence identity between two amino acid sequences, the skilled person may take into account so-called ‘conservative’ amino acid substitutions, which can generally be described as amino acid substitutions in which an amino acid residue is replaced with another amino acid residue of similar chemical structure and which has little or essentially no influence on the function, activity or other biological properties of the polypeptide. Possible conservative amino acid substitutions will be clear to the person skilled in the art.

As used herein, the term “antibody” refers to polyclonal antibodies, monoclonal antibodies, humanized antibodies, chimeric antibodies, minibodies, diabodies, nanobodies, nanoantibodies, affibodies, alphabodies, designed ankyrin-repeat domains, anticalins, knottins, engineered CH2 domains, single-chain antibodies, or fragments thereof such as Fab F(ab)2, F(ab)3, scFv, a single domain antibody, a heavy chain variable domain of an antibody, a heavy chain variable domain of a heavy chain antibody (VHH), the variable domain of a camelid heavy chain antibody, a variable domain of the a new antigen receptor (vNAR), a variable domain of a shark new antigen receptor, or other fragments or antibody formats that retain the antigen-binding function of a parent antibody. As such, an antibody may refer to an immunoglobulin, or fragment or portion thereof, or to a construct comprising an antigen-binding portion comprised within a modified immunoglobulin-like framework, or to an antigen-binding portion comprised within a construct comprising a nonimmunoglobulin-like framework or scaffold.

As used herein, the term “monoclonal antibody” refers to an antibody composition having a homogeneous antibody population. The term is not limited regarding the species or source of the antibody, nor is it intended to be limited by the manner in which it is made. The term encompasses whole immunoglobulins as well as fragments such as Fab, F(ab)2, Fv, and others that retain the antigen binding function of the antibody. Monoclonal antibodies of any mammalian species can be used in this invention. In practice, however, the antibodies will typically be of rat or murine origin because of the availability of rat or murine cell lines for use in making the required hybrid cell lines or hybridomas to produce monoclonal antibodies. As used herein, the term “polyclonal antibody” refers to an antibody composition having a heterogeneous antibody population. Polyclonal antibodies are often derived from the pooled serum from immunized animals or from selected humans.

“Heavy chain variable domain of an antibody or a functional fragment thereof” (also indicated hereafter as VHH), as used herein, means (i) the variable domain of the heavy chain of a heavy chain antibody, which is naturally devoid of light chains, including but not limited to the variable domain of the heavy chain of heavy chain antibodies of camelids or sharks or (ii) the variable domain of the heavy chain of a conventional four-chain antibody (also indicated hereafter as VH), including but not limited to a camelized (as further defined herein) variable domain of the heavy chain of a conventional four-chain antibody (also indicated hereafter as camelized VH).

As used herein, the terms “complementarity determining region” or “CDR” within the context of antibodies refer to variable regions of either the H (heavy) or the L (light) chains (also abbreviated as VH and VL, respectively) and contain the amino acid sequences capable of specifically binding to antigenic targets. These CDR regions account for the basic specificity of the antibody for a particular antigenic determinant structure. Such regions are also referred to as “hypervariable regions.” The CDRs represent non-contiguous stretches of amino acids within the variable regions but, regardless of species, the positional locations of these critical amino acid sequences within the variable heavy and light chain regions have been found to have similar locations within the amino acid sequences of the variable chains. The variable heavy and light chains of all canonical antibodies each have 3 CDR regions, each non-contiguous with the others (termed L1, L2, L3, H1, H2, H3) for the respective light (L) and heavy (H) chains.

As further described hereinbelow, the amino acid sequence and structure of a heavy chain variable domain of an antibody can be considered, without however being limited thereto, to be comprised of four framework regions or “FR's”, which are referred to in the art and hereinbelow as “framework region 1” or “FR1”; as “framework region 2” or “FR2”; as “framework region 3” or “FR3”; and as “framework region 4” or “FR4”, respectively, which framework regions are interrupted by three complementary determining regions or “CDR's”, which are referred to in the art as “complementarity determining region 1” or “CDR1”; as “complementarity determining region 2” or “CDR2”; and as “complementarity determining region 3” or “CDR3”, respectively.

As also further described hereinbelow, the total number of amino acid residues in a heavy chain variable domain of an antibody (including a V_{HH} or a V_{H}) can be in the region of 110-130, is preferably 112-115, and is most preferably 113. It should however be noted that parts, fragments or analogs of a heavy chain variable domain of an antibody are not particularly limited as to their length and/or size, as long as such parts, fragments or analogs retain (at least part of) the functional activity, such as the pesticidal, biocidal, biostatic activity, fungicidal or fungistatic activity (as defined herein) and/or retain (at least part of) the binding specificity of the original a heavy chain variable domain of an antibody from which these parts, fragments or analogs are derived from. Parts, fragments or analogs retaining (at least part of) the functional activity, such as the pesticidal, biocidal, biostatic activity, fungicidal or fungistatic activity (as defined herein) and/or retaining (at least part of) the binding specificity of the original heavy chain variable domain of an antibody from which these parts, fragments or analogs are derived from are also further referred to herein as “functional fragments” of a heavy chain variable domain.

A method for numbering the amino acid residues of heavy chain variable domains is the method described by Chothia et al. (Nature 342, 877-883 (1989)), the so-called “AbM definition” and the so-called “contact definition”. Herein, this is the numbering system adopted.

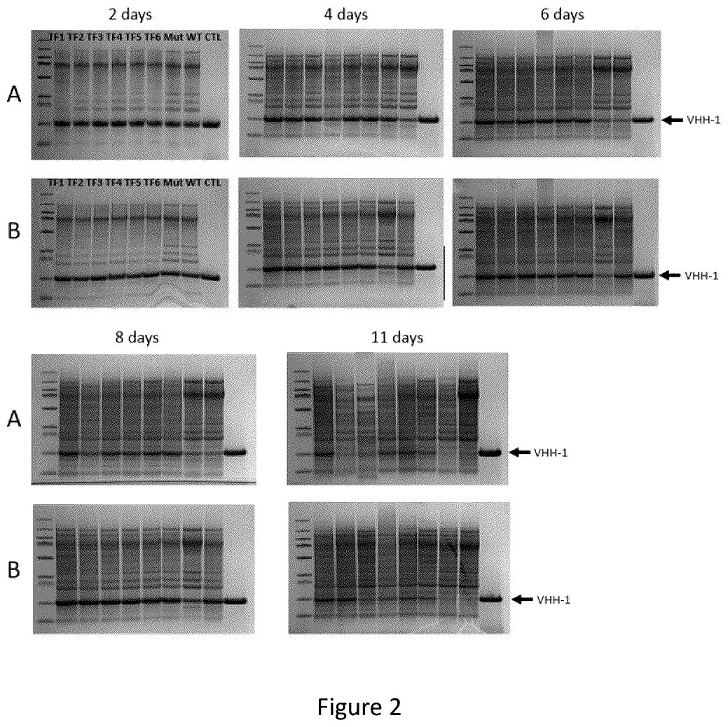

Alternatively, the amino acid residues of a variable domain of a heavy chain variable domain of an antibody (including a V_{HH} or a V_{H}) may be numbered according to the general numbering for heavy chain variable domains given by Kabat et al. (“Sequence of proteins of immunological interest”, US Public Health Services, NIH Bethesda, Md., Publication No. 91), as applied to V_{HH} domains from Camelids in the article of Riechmann and Muyldermans, referred to above (see for example FIG. 2 of said reference). For a general description of heavy chain antibodies and the variable domains thereof, reference is inter alia made to the following references, which are mentioned as general background art: WO 94/04678, WO 95/04079 and WO 96/34103 of the Vrije Universiteit Brussel; WO 94/25591, WO 99/37681, WO 00/40968, WO 00/43507, WO 00/65057, WO 01/40310, WO 01/44301, EP 1134231 and WO 02/48193 of Unilever; WO 97/49805, WO 01/21817, WO 03/035694, WO 03/054016 and WO 03/055527 of the Vlaams Instituut voor Biotechnologie (VIB); WO 03/050531 of Algonomics N.V. and Ablynx NV; WO 01/90190 by the National Research Council of Canada; WO 03/025020 (=EP 1 433 793) by the Institute of Antibodies; as well as WO 04/041867, WO 04/041862, WO 04/041865, WO 04/041863, WO 04/062551 by Ablynx; Hamers-Casterman et al., Nature 1993 Jun. 3; 363 (6428): 446-8.

Generally, it should be noted that the term “heavy chain variable domain” as used herein in its broadest sense is not limited to a specific biological source or to a specific method of preparation. For example, as will be discussed in more detail below, the heavy chain variable domains of the invention can be obtained (1) by isolating the V_{HH} domain of a naturally occurring heavy chain antibody; (2) by isolating the V_{H} domain of a naturally occurring four-chain antibody (3) by expression of a nucleotide sequence encoding a naturally occurring V_{HH} domain; (4) by expression of a nucleotide sequence encoding a naturally occurring V_{H} domain (5) by “camelization” (as described below) of a naturally occurring V_{H} domain from any animal species, in particular a species of mammal, such as from a human being, or by expression of a nucleic acid encoding such a camelized V_{H} domain; (6) by “camelisation” of a “domain antibody” or “Dab” as described by Ward et al (supra), or by expression of a nucleic acid encoding such a camelized V_{H} domain (7) using synthetic or semi-synthetic techniques for preparing proteins, polypeptides or other amino acid sequences; (8) by preparing a nucleic acid encoding a V_{HH} or a V_{H} using techniques for nucleic acid synthesis, followed by expression of the nucleic acid thus obtained; and/or (9) by any combination of the foregoing. Suitable methods and techniques for performing the foregoing will be clear to the skilled person based on the disclosure herein and for example include the methods and techniques described in more detail hereinbelow.

However, according to a specific embodiment, the heavy chain variable domains as disclosed herein do not have an amino acid sequence that is exactly the same as (i.e. as a degree of sequence identity of 100% with) the amino acid sequence of a naturally occurring V_{H} domain, such as the amino acid sequence of a naturally occurring V_{H} domain from a mammal, and in particular from a human being.

The term “affinity”, as used herein, refers to the degree to which a polypeptide, in particular an immunoglobulin, such as an antibody, or an immunoglobulin fragment, such as a V_{HH}, binds to an antigen so as to shift the equilibrium of antigen and polypeptide toward the presence of a complex formed by their binding. Thus, for example, where an antigen and antibody (fragment) are combined in relatively equal concentration, an antibody (fragment) of high affinity will bind to the available antigen so as to shift the equilibrium toward high concentration of the resulting complex. The dissociation constant is commonly used to describe the affinity between the protein binding domain and the antigenic target. Typically, the dissociation constant is lower than 10-5 M. Preferably, the dissociation constant is lower than 10-6 M, more preferably, lower than 10-7 M. Most preferably, the dissociation constant is lower than 10-8 M.

The terms “specifically bind” and “specific binding”, as used herein, generally refers to the ability of a polypeptide, in particular an immunoglobulin, such as an antibody, or an immunoglobulin fragment, such as a V_{HH}, to preferentially bind to a particular antigen that is present in a homogeneous mixture of different antigens. In certain embodiments, a specific binding interaction will discriminate between desirable and undesirable antigens in a sample, in some embodiments more than about 10 to 100-fold or more (e.g., more than about 1000—or 10,000-fold).

Accordingly, an amino acid sequence as disclosed herein is said to “specifically bind to” a particular target when that amino acid sequence has affinity for, specificity for and/or is specifically directed against that target (or for at least one part or fragment thereof).

The “specificity” of an amino acid sequence as disclosed herein can be determined based on affinity and/or avidity.

An amino acid sequence as disclosed herein is said to be “specific for a first target antigen of interest as opposed to a second target antigen of interest” when it binds to the first target antigen of interest with an affinity that is at least 5 times, such as at least 10 times, such as at least 100 times, and preferably at least 1000 times higher than the affinity with which that amino acid sequence as disclosed herein binds to the second target antigen of interest. Accordingly, in certain embodiments, when an amino acid sequence as disclosed herein is said to be “specific for” a first target antigen of interest as opposed to a second target antigen of interest, it may specifically bind to (as defined herein) the first target antigen of interest, but not to the second target antigen of interest.

“Fungicidal activity”, as used herein, means to interfere with the harmful activity of a fungus, including but not limited to killing the fungus, inhibiting the growth or activity of the fungus, altering the behavior of the fungus, and repelling or attracting the fungus.

“Fungistatic activity”, as used herein, means to interfere with the harmful activity of a fungus, including but not limited to inhibiting the growth or activity of the fungus, altering the behavior of the fungus, and repelling or attracting the fungus.

“Culturing”, “cell culture”, “fermentation”, “fermenting” or “microbial fermentation” as used herein means the use of a microbial cell to produce a compound of interest, such as a polypeptide, at an industrial scale, laboratory scale or during scale-up experiments. It includes suspending the microbial cell in a broth or growth medium, providing sufficient nutrients including but not limited to one or more suitable carbon source (including glucose, sucrose, fructose, lactose, avicel®, xylose, galactose, ethanol, methanol, or more complex carbon sources such as molasses or wort), nitrogen source (such as yeast extract, peptone or beef extract), trace element (such as iron, copper, magnesium, manganese or calcium), amino acid or salt (such as sodium chloride, magnesium chloride or natrium sulfate) or a suitable buffer (such as phosphate buffer, succinate buffer, HEPES buffer, MOPS buffer or Tris buffer). Optionally it includes one or more inducing agents driving expression of the compound of interest or a compound involved in the production of the compound of interest (such as lactose, IPTG, ethanol, methanol, sophorose or sophorolipids). If can also further involve the agitation of the culture media via for example stirring of purging to allow for adequate mixing and aeration. It can further involve different operational strategies such as batch cultivation, semi-continuous cultivation or continuous cultivation and different starvation or induction regimes according to the requirements of the microbial cell and to allow for an efficient production of the compound of interest or a compound involved in the production of the compound of interest. Alternatively, the microbial cell is grown on a solid substrate in an operational strategy commonly known as solid state fermentation.

Fermentation broth, culture media or cell culture media as used herein can mean the entirety of liquid or solid material of a fermentation or culture at any time during or after that fermentation or culture, including the liquid or solid material that results after optional steps taken to isolate the compound of interest. As such, the fermentation broth or culture media as defined herein includes the surroundings of the compound of interest after isolation of the compound of interest, during storage and/or during use as an agrochemical or pharmaceutical composition. Fermentation broth is also referred to herein as a culture medium or cell culture medium.

“Peptone” as used herein means a “protein hydrolysate”, which is any water-soluble mixture of polypeptides and amino acids formed by the partial hydrolysis of protein. More specifically “peptone” or “protein hydrolysate” are the water-soluble products derived from the partial hydrolysis of protein rich starting material which can be derived from plant, yeast, or animal sources. Typically, “peptone” or “protein hydrolysate” are produced by a protein hydrolysis process accomplished using strong acids, bases, or proteolytic enzymes. In more detail peptone or protein hydrolysates are produced by combining protein and demineralized water to form a thick suspension of protein material in large-capacity digestion vessels, which are stirred continuously throughout the hydrolysis process. For acid or basic hydrolysis, the temperature is adjusted, and the digestion material added to the vessel. For proteolytic digestion, the protein suspension is adjusted to the optimal pH and temperature for the specific enzyme or enzymes chosen for the hydrolysis. The desired degree of hydrolysis depends on the amount of enzyme, time for digestion, and control of pH and temperature. A typical “peptone” or “protein hydrolysate” may comprise about 25% polypeptides, about 30% free amino acids, about 20% carbohydrates, about 15% salts and trace metals and about 10% vitamins, organic acids, and organic nitrogen bases. Depending on the starting material “peptone” or “protein hydrolysate” can be completely free of animal-derived products and/or GMO products. For example, “Peptone” or “protein hydrolysate” can be produced using high quality pure protein as a starting material. Alternatively, “peptone” or “protein hydrolysate” can be produced by using soymeal as a starting material. When soymeal is used as a starting material this soymeal can be free of animal sources. This soymeal can furthermore be free of GMO material. This soymeal can be defatted soya. Alternatively, “peptone” or “protein hydrolysate” can be produced by using casein as a starting material. Alternatively, “peptone” or “protein hydrolysate” can be produce by using milk as a starting material. Alternatively, “peptone” or “protein hydrolysate” can be produce by using meat paste as a starting material. When meat paste is used as a starting material this meat paste can be for example from bovine or porcine origin. When meat paste is used as a starting material this meat paste can be derived from organs, such as harts or alternatively for example muscle tissue. Alternatively, “peptone” or “protein hydrolysate” can be produced using gelatin as a starting material. Alternatively, “peptone” or “protein hydrolysate” can be produced by using yeast as a starting material.

Accordingly, in some embodiments, the peptone is the product of partial hydrolysis of plant, animal or yeast protein.

In some embodiments, the peptone is produced by acid hydrolysis, by base hydrolysis or by enzymatic digestion.

In some embodiments, the peptone comprises at least about 5% polypeptides (weight/weight %). For example, in some embodiments the peptone comprises from about 5% to about 50% (weight/weight %) polypeptides.

In some embodiments, the peptone comprises at least about 5% (weight/weight %) free amino acids. For example, in some embodiments the peptone comprises from about 5% to about 50% (weight/weight %) free amino acids.

In some embodiments, the peptone comprises at least about 5% (weight/weight %) salts. For example, in some embodiments the peptone comprises from about 5% to about 20% (weight/weight %) salts.

In some embodiments, the peptone comprises at least about 5% (weight/weight %) carbohydrates. For example, in some embodiments the peptone comprises from about 5% to about 40% (weight/weight %) carbohydrates.

In some embodiments, the peptone comprises at least about 5% (weight/weight %) carbohydrates about 5% (weight/weight %) vitamins, organic acids, and organic nitrogen bases. For example, in some embodiments the peptone comprises from about 5% to about 20% (weight/weight %) vitamins, organic acids, and organic nitrogen bases.

In some embodiments, the peptone comprises from at least about 5% (weight/weight %) of polypeptides, at least about 5% (weight/weight %) free amino acids, at least about 5% (weight/weight %) salts, at least about 5% (weight/weight %) carbohydrates and at least about 5% (weight/weight %) in total of vitamins, organic acids, and organic nitrogen bases.

In some embodiments, the peptone comprises from about 15% to about 35% (weight/weight %) polypeptides, from about 20% to about 40% (weight/weight %) free amino acids, from about 10% to about 30% (weight/weight %) carbohydrates, from about 5% to about 25% (weight/weight %) salts, and from about 5% to about 15% (weight/weight %) in total of vitamins, organic acids, and organic nitrogen bases. Of course the skilled person will be aware the total amount cannot exceed 100% when all components are added together. The peptone may comprise additional components not specifically listed here. In some embodiments, the peptone is free of animal derived products.

In some embodiments, the peptone is the product of partial hydrolysis of soymeal, casein, milk, meat, gelatine, or yeast.

Culturing in the presence of peptone means the cell culture medium comprise peptone. The peptone may be present at any suitable concentration. For example, in some embodiments the peptone concentration may be from about 1 g/L to about 100 g/L, for example from about 10 g/L to about 80 g/L, for example about 20 g/L, about 30 g/L, about 40 g/L, about 50 g/L, about 60 g/L, or about 70 g/L. The cell culture medium used for culture of the microbial host cell may already comprise peptone. Alternatively, the cell culture medium may be modified to include peptone. For example, peptone may be added to the cell culture medium at any suitable time during the culturing of the microbial cell. For example, in embodiments where the compound of interest is encoded by a nucleotide that is operably linked to an inducible promoter, the peptone may be added to the cell culture medium in the fermentation chamber at the same time as or shortly after expression of the compound of interest is induced. Alternatively, the peptone may be added to the cell culture medium in the fermentation chamber before induction of expression of the compound of interest.

In embodiments where the cell culture medium does not already comprise peptone and this must be added to the cell culture medium, this may be added to the cell culture medium before adding the cell culture medium to the fermentation chamber. Alternatively, the peptone may be added to the fermentation chamber separately, preferably after the cell culture medium is added to the fermentation chamber.

“Isolating the compound of interest” is an optional step or series of steps taking the cell culture media or fermentation broth as an input and increasing the amount of the compound of interest relative to the amount of culture media or fermentation broth. Isolating the compound of interest may alternatively or additionally comprises obtaining or removing the compound of interest form the culture media or fermentation broth. Isolating the compound of interest can involve the use of one or multiple combinations of techniques well known in the art, such as precipitation, centrifugation, sedimentation, filtration, diafiltration, affinity purification, size exclusion chromatography and/or ion exchange chromatography. In some embodiments, isolating the compound of interest may comprise a step of lysing the microbial cells to release the compound of interest, for example if the compound of interest is not secreted by the microbial cells, or at least is not secreted by the microbial cells to a significant enough degree. Isolating the compound of interest may be followed by formulation of the compound of interest into an agrochemical or pharmaceutical composition.

The term “yield” as used herein refers to the amount of a compound of interest produced. When using the term “improved” or “increased” or a similar term when referring to “yield”, it is meant that the compound of interest produced by the modified microbial host cell of the invention capable of producing a compound of interest is increased in quantity, quality, stability and/or concentration either in the fermentation broth or cell culture media, as a purified or partially purified compound, during storage and/or during use as an agrochemical or pharmaceutical composition. The increase in yield is compared to the yield of compound of interest produced by a microbial host cell that has not been modified to affect the production, stability and/or function of at least one polypeptide, i.e. the parental microbial host cell.

In some embodiments, the yield is increased by at least about 1%, at least about 2%, at least about 3%, at least about 4%, at least about 5%, at least about 6%, at least about 7%, at least about 8%, at least about 9%, at least about 10%, at least about 20%, at least about 30%, at least about 40%, at least about 50%, at least about 60%, at least about 70%, at least about 80%, at least about 90% or at least about 100%, at least about 110%, at least about 120%, at least about 130%, at least about 140%, at least about 150%, at least about 160%, at least about 170%, at least about 180%, at least about 190%, at least about 200%, at least about 210%, at least about 220%, at least about 230%, at least about 240%, at least about 250%, at least about 260%, at least about 270%, at least about 280%, at least about 290% or at least about 300%, at least about 500%, at least about 1000% or at least about 1500% when a modified microbial host cell is used to produce the compound of interest, compared to a parental microbial host cell (the parental host cell only having been modified to express the compound of interest).

“Agrochemical”, “agrochemically” or “agrochemically suitable” as used herein, means suitable for use in the agrochemical industry (including agriculture, horticulture, floriculture and home and garden uses), but also products intended for non-crop related uses such as public health/pest control operator uses to control undesirable insects and rodents, household uses, such as household fungicides and insecticides and agents, for protecting plants or parts of plants, crops, bulbs, tubers, fruits (e.g. from harmful organisms, diseases or pests); for controlling, preferably promoting or increasing, the growth of plants; and/or for promoting the yield of plants, crops or the parts of plants that are harvested (e.g. its fruits, flowers, seeds etc.). Examples of such substances will be clear to the skilled person and may for example include compounds that are active as insecticides (e.g. contact insecticides or systemic insecticides, including insecticides for household use), herbicides (e.g. contact herbicides or systemic herbicides, including herbicides for household use), fungicides (e.g. contact fungicides or systemic fungicides, including fungicides for household use), nematicides (e.g. contact nematicides or systemic nematicides, including nematicides for household use) and other pesticides or biocides (for example agents for killing insects or snails); as well as fertilizers; growth regulators such as plant hormones; micro-nutrients, safeners, pheromones; repellants; insect baits; and/or active principles that are used to modulate (i.e. increase, decrease, inhibit, enhance and/or trigger) gene expression (and/or other biological or biochemical processes) in or by the targeted plant (e.g. the plant to be protected or the plant to be controlled), such as nucleic acids (e.g., single stranded or double stranded RNA, as for example used in the context of RNAi technology) and other factors, proteins, chemicals, etc. known per se for this purpose, etc. Examples of such agrochemicals will be clear to the skilled person; and for example include, without limitation: glyphosate, paraquat, metolachlor, acetochlor, mesotrione, 2,4-D, atrazine, glufosinate, sulfosate, fenoxaprop, pendimethalin, picloram, trifluralin, bromoxynil, clodinafop, fluroxypyr, nicosulfuron, bensulfuron, imazetapyr, dicamba, imidacloprid, thiamethoxam, fipronil, chlorpyrifos, deltamethrin, lambda-cyhalotrin, endosulfan, methamidophos, carbofuran, clothianidin, cypermethrin, abamectin, diflufenican, spinosad, indoxacarb, bifenthrin, tefluthrin, azoxystrobin, thiamethoxam, tebuconazole, mancozeb, cyazofamid, fluazinam, pyraclostrobin, epoxiconazole, chlorothalonil, copper fungicides, trifloxystrobin, prothioconazole, difenoconazole, carbendazim, propiconazole, thiophanate, sulphur, boscalid and other known agrochemicals or any suitable combination(s) thereof.

An “agrochemical composition”, as used herein means a composition for agrochemical use, as further defined, comprising at least one active substance, optionally with one or more additives (for example one or more additives favoring optimal dispersion, atomization, deposition, leaf wetting, distribution, retention and/or uptake of agrochemicals). It will become clear from the further description herein that an agrochemical composition as used herein includes biological control agents or biological pesticides (including but not limited to biological biocidal, biostatic, fungistatic and fungicidal agents) and these terms will be interchangeably used in the present application. Accordingly, an agrochemical composition as used herein includes compositions comprising at least one biological molecule as an active ingredient, substance or principle for controlling pests in plants or in other agro-related settings (such for example in soil). Non-limiting examples of biological molecules being used as active principles in the agrochemical compositions disclosed herein are proteins (including antibodies and fragments thereof, such as but not limited to heavy chain variable domain fragments of antibodies, including VHH's), nucleic acid sequences, (poly-) saccharides, lipids, vitamins, hormones glycolipids, sterols, and glycerolipids. As a non-limiting example, the additives in the agrochemical compositions disclosed herein may include but are not limited to excipients, diluents, solvents, adjuvants, surfactants, wetting agents, spreading agents, oils, stickers, thickeners, penetrants, buffering agents, acidifiers, anti-settling agents, anti-freeze agents, photo-protectors, defoaming agents, biocides and/or drift control agents. The compound of interest may be formulated with one or more such components when preparing an agrochemical composition. For example, the compound of interest may be formulated with one or more additives, for example one or more agrochemically acceptable excipients.

A “pharmaceutical composition”, “pharmaceutically” or “pharmaceutically suitable” as used herein means a composition for medical use. For example, the composition may be suitable for injection or infusion which can include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form must be sterile, fluid, and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin. The compound of interest may be formulated with one or more such components when preparing a pharmaceutical composition. For example, the compound of interest may be formulated with one or more additives, for example one or more pharmaceutically acceptable excipients.

The present invention also provides method of producing a microbial host cell according to the invention. The methods may comprise providing a parent microbial host cell; and modifying the parent microbial host cell according to techniques described herein, wherein the modification affects the production, stability and/or function of the at least one polypeptide. The step of modifying the parent microbial host cell comprises targeting the at least one polypeptide, its corresponding chromosomal gene and/or its corresponding mRNA by anti-sense techniques, RNAi techniques, CRISPR techniques, a small molecule inhibitor, an antibody, an antibody fragment or a combination thereof. The method may further comprise inserting a polynucleotide coding for a compound of interest into the microbial host cell.

In some embodiments, the method comprises providing a modified microbial host cell of the invention, and inserting a polynucleotide coding for a compound of interest into the microbial host cell. Therefore, the methods may begin from a parental microbial host cell, and include the steps of modifying the microbial host cell to modulate the protease activity of the microbial host cell, or the methods may begin from an already modified microbial host cell and comprise a step of inserting a polynucleotide coding for a compound of interest into the microbial host cell.

In some embodiments, there is provided a modified filamentous fungi host cell which is characterized by (a) having been modified and where this modification adversely affects the production, stability and/or function of at least one regulator of transcription; and (b) having a reduction or deficiency in protease activity if compared with a parent microbial host cell which has not been modified and is measured under the same or substantially the same conditions; wherein the at least one regulator of transcription is a promoter of transcription that causes, promotes or initiates the expression of one or more proteases. Alternatively, there is provided a modified filamentous fungi host cell which is characterized by (a) having been modified and where this modification positively affects the production, stability and/or function of at least one regulator of transcription; and (b) having a reduction or deficiency in protease activity if compared with a parent microbial host cell which has not been modified and is measured under the same or substantially the same conditions; wherein the at least one regulator of transcription is a repressor of transcription that interrupts, represses or halts the expression of one or more proteases. The modified microbial host cell may further comprise a polynucleotide encoding a compound of interest, where in the compound of interest is an antibody or functional fragment thereof (such as a VHH), wherein the polynucleotide encoding the compound of interest is operably linked to a promoter. In preferred embodiments, the regulator of transcription may have an amino acid sequence according to SEQ ID NO: 1, SEQ ID NO: 28, SEQ ID NO: 33, SEQ ID NO: 36, SEQ ID NO: 58 or SEQ ID NO: 59, or a sequence at least about 80%, at least about 85%, at least about 90%, at least about 95%, at least about 98%, or at least 99% identical thereto.

### Method of Making Compounds of Interest

The invention provides methods for the production of a compound of interest. The compound of interest may be a compound as described herein, for example an antibody or a functional fragment thereof, a carbohydrate-binding domain, a heavy chain antibody or a functional fragment thereof, a single domain antibody, a heavy chain variable domain of an antibody or a functional fragment thereof, a heavy chain variable domain of a heavy chain antibody or a functional fragment thereof, a variable domain of camelid heavy chain antibody (VHH) or a functional fragment thereof, a variable domain of a new antigen receptor, a variable domain of shark new antigen receptor (vNAR) or a functional fragment thereof, a minibody, a nanobody, a nanoantibody, an affibody, an alphabody, a designed ankyrin-repeat domain, an anticalins, a knottins or an engineered CH2 domain. In some embodiments, the compound of interest is an antibody, for example a V_{HH}. The methods comprise providing a modified microbial host cell of the invention, which is characterized by (a) having been modified and where this modification affects the production, stability and/or function of at least one polypeptide; and having a reduction or deficiency in protease activity if compared with a parent microbial host cell which has not been modified and is measured under the same or substantially the same conditions. The host cell is capable of expressing the compound of interest. The method further comprises culturing said modified microbial host cell under conditions conducive to the expression of the compound of interest. The method may further optionally comprise a step of isolating the compound of interest from the culture medium or fermentation broth.

The modified microbial host cell that is provided may already be capable of expressing the compound of interest. For example, the modified microbial host cell may be provided already comprising a polynucleotide coding for the compound of interest, and the sequence encoding the compound of interest may be operable linked to a promoter (for example a constitutive promoter or an inducible promoter). Alternatively, the method may comprise a step of transforming the microbial host cell with the polynucleotide to insert the polynucleotide into the microbial host cell. The step of transforming the microbial host cell, if present, may occur before, after or simultaneously with the modification of the microbial host cell to modify the production, structure and/or function of the at least one polypeptide.

In some embodiments, the methods may comprise a step of inducing expression of the compound of interest by the microbial host cell. For example, if the compound of interest is encoded by a nucleotide sequence that is operably linked to an inducible promoter, the method may comprise a step of inducing the expression of the compound of interest. A common inducible promoter that may be used is the inducible cbh1 or cbh2 promoter, in which administration of lactose will initiate expression. Other inducible promoters could of course be used. If the sequence encoding the compound of interest is under the control of a constitutive promoter, no specific step of induction of expression may be required.

Fermentation or culture of the microbial host cells may occur in a solid fermentation or culture setting or a liquid fermentation or culture setting. Solid-state fermentation or culture may comprise seeding the microbial host cell on a solid culture substrate, and methods of solid-state fermentation or culture are known the skilled person. Liquid fermentation or culture may comprise culturing the microbial host cell in a liquid cell culture medium.

The method may also comprise a step of isolating the compound of interest produced by the microbial host cell, for example isolating the compound of interest from the fermentation broth or cell culture medium.

The method may further comprise a step of formulating the compound of interest into a agrochemical or pharmaceutical composition. The step of formulating the compound of interest into an agrochemical composition may comprise formulating the compound of interest with one or more agrochemically acceptable excipients. The step of formulating the compound of interest into a pharmaceutical composition may comprise formulating the compound of interest with one or more pharmaceutically acceptable excipients.

The present invention therefore provides compounds of interest obtained by a method of the present invention. The present invention also therefore provides an agrochemical or pharmaceutical composition obtained by a method of the present invention.

The present invention also provides the use of a modified microbial host cell of the invention for the production of a compound of interest, wherein the microbial host cell is characterized by (a) having been modified and where this modification affects the production, stability and/or function of at least one polypeptide; (b) having a reduction or deficiency in protease activity if compared with a parent microbial host cell which has not been modified and is measured under the same or substantially the same conditions; and (c) comprising at least one polynucleotide coding for the compound of interest.

Any methods comprising or requiring the culturing or fermentation of the modified microbial host cell comprise the culture or fermentation of the host cell is a suitable medium. Generally, the medium will comprise any and all nutrients required for the microbial host cell to grow. The skilled person will be aware of the required components of the cell culture media or fermentation broth, which may differ depending on the species of microbial host cell being cultured. In some embodiments, the cell culture media or fermentation broth may comprise a nitrogen source, such as ammonium or peptone.

### Kit of Parts

The present invention also provides a kit of parts. The kit comprises a modified microbial host cell which is characterized by (a) having been modified and where this modification affects the production, stability and/or function of at least one polypeptide; and having a modulation in protease activity if compared with a parent microbial host cell which has not been modified and is measured under the same or substantially the same conditions.

The host cell may be capable of expressing a compound of interest. For example, the microbial host cell may comprise a polynucleotide coding for the compound of interest. Alternatively or additionally, the kit may further comprise a vector, wherein the vector comprises a polynucleotide sequence coding for the compound of interest. The vector may comprise a promoter that is operable linked to the polynucleotide sequence coding for the compound of interest.

The kit may further comprise a vector for homologous recombination, for example for effecting a full or partial deletion of a gene encoding the at least one polypeptide in the microbial cell, or for effecting the inactivation of a gene encoding the at least one polypeptide in the microbial cell. In such embodiments, the microbial cell of the kit may not be modified and therefore it may be a parental or wild-type microbial host cell. The vector for effecting a full or partial deletion or the inactivation of the gene encoding the at least one polypeptide in the microbial cell may be a vector for full or partial deletion of the gene encoding the at least one polypeptide, for example by restriction enzyme techniques such as CRIPSR. The vector may comprise a sequence comprising, in a 5′ to 3′ direction, a first flanking homology arm, a reporter gene and a second flanking homology arm. Optionally, the first flanking homology arm, the reporter gene and the second flanking homology arm may be flanked by restriction sites. Reporter genes are selectable and/or counter-selectable genetic markers that allow the selection of microbial host cells that have been correctly modified. The vector may alternatively serve as a template for a PCR for amplifying at least the first flanking homology arm, a reporter gene and a second flanking homology arm. The resulting linear dsDNA product can then be used for homologous recombination. A linear dsDNA product can alternatively be generated by cutting the restriction sites flanking the first flanking homology arm, a reporter gene and a second flanking homology arm can with the corresponding restriction enzymes. The resulting linear dsDNA product can then be used for homologous recombination. The homology arms may be complimentary or substantially complimentary to the gene encoding the at least one polypeptide in the microbial cell. For example, the first flanking arm may be complementary to a sequence upstream, adjacent to and/or overlapping with one end of the gene encoding the polypeptide in the microbial host cell, and the second flanking arm may be complementary to a sequence downstream, adjacent to and/or overlapping with the other end of the gene encoding the polypeptide in the microbial host cell. The two flanking arms therefore flank all or part of the gene encoding the at least one polypeptide in the microbial host cell. As such, the insertion of the reporter gene into the microbial host cell and the deletion or disruption of the gene encoding the polypeptide in the microbial cell can be achieved. The sequence that is flanked by the two homology arms in the vector may comprise a portion of the sequence encoding the polypeptide, sequence that is flanked by the two homology arms in the vector may comprise a modified version of the gene encoding the polypeptide, wherein the modified version has a reduced or altered function compared to an unmodified version of the gene encoding the polypeptide (for example an insertion, substitution or deletion mutant that adversely affect the activity of the polypeptide when expressed in the microbial host cell).

Each vector may have one or more antibiotic resistance genes. For example, the vectors may have an antibiotic resistance gene to enable the selection of microbial host cells which have been transformed with the vectors. When the microbial host cell is to be transformed using 2 vectors, the 2 vectors may comprise different antibiotic resistance genes, to enable the selection of microbial host cells that have been transformed with both vectors.

In one embodiment, the kit comprises:
a) a parental microbial host cell; and
b) a vector for homologous recombination, for example for effecting a full or partial deletion of a gene encoding at least one polypeptide in the microbial cell, or for effecting the inactivation of a gene encoding the at least one polypeptide in the microbial cell, where the at least one polypeptide is a regulator of transcription that controls the expression of one or more proteases; and optionally further comprises
c) a vector comprising a nucleotide sequence coding for a compound of interest, wherein the nucleotide sequence is operably linked to a promoter.

In one embodiment, the kit comprises:
a) a parental microbial host cell comprising a polynucleotide coding for a compound of interest, wherein the nucleotide sequence is operably linked to a promoter; and
b) a vector for homologous recombination, for example for effecting a full or partial deletion of at least one polypeptide in the microbial cell, where the at least one polypeptide is a regulator of transcription that controls the expression of one or more proteases;

In one embodiment, the kit comprises:
a) a modified microbial host cell, wherein microbial host cell has been modified to adversely affects the production, stability and/or function of at least one regulator of transcription that controls the expression of one or more proteases; and
b) a vector comprising a nucleotide sequence coding for a compound of interest, wherein the nucleotide sequence is operably linked to a promoter.

In one embodiment, the kit comprises:
a) a vector for homologous recombination of a microbial cell, for example for effecting a full or partial deletion of at least one polypeptide encoded by the genome of the microbial cell, where the at least one polypeptide is a regulator of transcription that controls the expression of one or more proteases; and
b) a vector comprising a nucleotide sequence coding for a compound of interest, wherein the nucleotide sequence is operably linked to a promoter.

The different components of the kit may each be disposed separately in separate containers.

In some embodiments, the kit may further comprise instructions for use. The instructions may, for example, provide instructions for modifying the microbial host cell to affect the production, functional and/or stability of the one or more polypeptides. The instructions may alternatively or additionally provide instructions for producing a compound of interest using the microbial host cell. The instructions may also alternatively or additionally provide instructions for carrying out any of the methods of the invention disclosed herein.

The polypeptide that is targeted using an option vector of the kit is a polypeptide as described elsewhere herein with respect to the modified microbial host cells of the invention. Similarly, the vector comprising a nucleotide sequence coding for a compound of interest is a vector comprising a nucleotide sequence coding for a compound of interest as described elsewhere herein with respect to the modified microbial host cells of the invention.

The Figures, Sequence Listing and the Experimental Part/Examples are only given to further illustrate the invention and should not be interpreted or construed as limiting the scope of the invention and/or of the appended claims in any way, unless explicitly indicated otherwise herein.

The above disclosure will now be further described by means of the following non-limiting examples.

### EXAMPLES

The following non-limiting Examples describe methods and means according to the invention. Unless stated otherwise in the Examples, all techniques are carried out according to protocols standard in the art.

The following examples are included to illustrate embodiments of the invention. Those of skill in the art should, in light of the present disclosure, appreciate that many changes can be made in the specific embodiments which are disclosed and still obtain a like or similar result without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### Example 1: Microbial Host Cell Proteins and Genes

The identified microbial host cell, specifically Trichoderma reesei RL-P37 (NRRL 15709), target polypeptide, Are1, may be defined by peptide sequence (SEQ ID NO: 1), genomic DNA sequence (SEQ ID NO: 2), and/or coding DNA sequence (SEQ ID NO: 3).

### Example 2: Generation of Genomic Modifications in Microbial Host Cells

The Are1 gene was deleted from the genome of Trichoderma reesei RL-P37 (NRRL 15709). To obtain the fragments necessary to assemble the deletion cassettes, genomic DNA from T. reesei was extracted using the Wizard Genomic DNA purification kit (Promega-A1120) according to the manufacturer's instructions. The pellet was resuspended in 60 μL of DNA Rehydration Solution by incubating at 65° C. for 1 hour. T. reesei genomic DNA was obtained in sufficient quantity and quality to perform subsequent experiments.

To construct the donor DNAs, the 5′ and 3′ flanking fragments of Are1 (1045 bp/1016 bp) were amplified separately using PCR. The selection marker expression cassette comprising the hygB gene (encoding hygromycin B phosphotransferase), under the control of the oliC promoter and the trpC terminator of Aspergillus nidulans (PoliC-hph-TtrpC) was obtained via gene synthesis and amplified separately with specific primers. The primer sequences used in this study are listed in Table 1.

TABLE 1
Primers used to obtain the DNA donors for the Are1 deletions

| Seq ID | Name | Sequence | Product size |
|---|---|---|---|
| 4 | 5′ Are1_fwd | ctcgagtttttcagcaagatACTAGTCCTGACCTTATTTCCCG | 1045 bp |
| 5 | 5′ Are1_rev | acagctgcagCTGCGGTCGGTACTTCGATG | |
| 6 | Are1-hyg_fwd | ccgaccgcagCTGCAGCTGTGGAGCCGC | 2302 bp |
| 7 | Are1-hyg_rev | tacgctgtctCATGACACATGCATCCACCATCG | |
| 8 | 3′ Are1_fwd | atgtgtcatgAGACAGCGTAGGCATAGC | 1016 bp |
| 9 | 3′ Are1_rev | aggagatcttctagaaagatACTAGTGGAAAGAAACGTAAAG | |

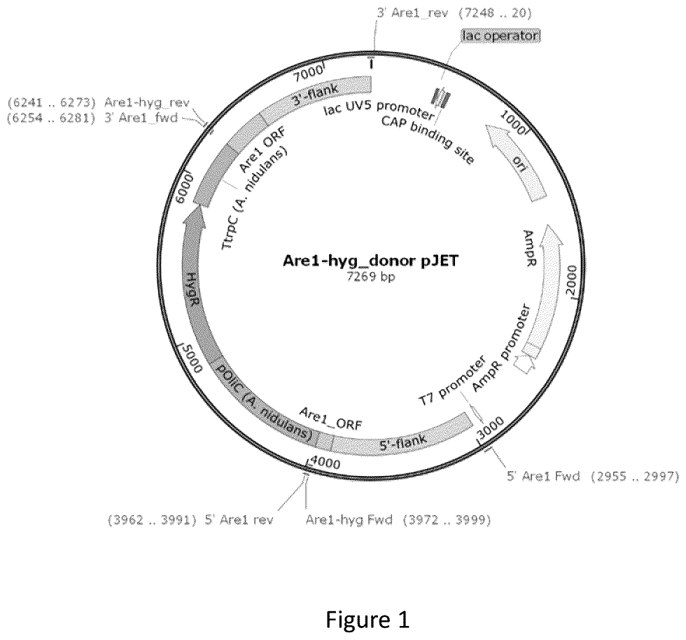

The PCR amplified 5′ Are1 flanking fragment, hygromycin selection marker and 3′ Are1 flanking region were assembled in cloning vector pJET 1.2 using the NEBuilder HiFi Assembly Master Mix, and E. coli DH5a competent cells were transformed with the ligation mixture to generate the plasmids containing the donor DNAs for the Are1 deletion, as shown in FIG. 1. The successful assembly of the Are1-hyg_donor pJET plasmid was confirmed by restriction enzyme digestion and sequencing using the primers shown in Table 2.

TABLE 2
Primers used for sequencing analysis

| Seq ID | Name | Sequence |
|---|---|---|
| 10 | Are1_seq1 | GATGAAAGTGGGATGAATGACAGGGA |
| 11 | Are1_seq2 | ATTCGGCGTGCACATCAAAGGCGT |
| 12 | pOliC_seq1 | CCGCTTAATTTTCGCCCTTTTTTCA |
| 13 | pOliC_seq2 | CTAGCGCACGAAAGACGCG |
| 14 | hyg_seq1 | AGAAGAAGATGTTGGCGACCT |
| 15 | hyg_seq2 | TCTTGACCAACTCTATCAGAGCTTG |
| 16 | Are1_seq3 | TCCCAGCATGCCTTCCATCTC |
| 17 | pJET_rev | GAGAATATTGTAGGAGATCTTCTAGA |

To obtain sufficient DNA for the T. reesei transformation, a PCR was performed with the primers as described in Table 3 using the Are1-hyg donor pJET plasmid as a template, resulting in the Are1 deletion cassette fragment.

TABLE 3
Primers used for the donor amplification

| Seq ID | Name | Sequence | Product size |
|---|---|---|---|
| 18 | Are1-hyg | ACTAGTCCTGACCTTATTTCCCGCA | 4295 bp |
| | dCassette_fwd | | |
| 19 | Are1-hyg | ACTAGTGGAAAGAAACGTAAAGTATCTG | |
| | dCassette_rev | | |

Transformation of protoplasts from Trichoderma reesei RL-P37 (NRRL 15709) with the Are1 deletion cassette fragment was carried out using a standard poly-ethylene glycol (PEG) mediated transformation method as described previously (Penttilä et al., 1987).

Successful transformants were selected on potato dextrose agar (Sigma-Aldrich) plates with 100 μg/mL hygromycin as the selective agent. The plates were incubated for 7 days until colonies could be transferred to secondary selection plates. Six colonies (labeled TF1 to TF6) were obtained. The hygromycin resistant colonies and parent microbial host cell were grown in 500 ml Vogel's liquid medium (100 mL=2 mL Vogel's 50× stock solution, 3 mL glucose (50% stock solution), 200 mL tween-80 (10% stock solution), 9.4 mL yeast extract (80 g/L stock solution)) and 100 μg/mL of hygromycin (in the case of transformants). Genomic DNA was extracted from colonies using Phire Plant Direct PCR Kit (Thermo Fisher Scientific). The resulting genomic DNA sample was diluted into 10 μl water, debris was removed by centrifugation, and the supernatant was used as a template in subsequent PCR.

Oligonucleotides were designed outside the flanking regions of the target locus to identify the possible integration of the donor cassettes into the deletion region (Table 4). The expected size of the hygromycin expression cassette was integrated into the upstream region of the Are1 target, observing 1957 bp of the amplicon size in the transformants examined. Likewise, in the downstream region an amplicon of 1667 bp was observed. In conclusion the Are1 gene was successfully deleted in all 6 transformants TF1 to TF6.

TABLE 4
Primers used for screening of donor cassette integration into
the deletion region

| | | | Product |
|---|---|---|---|
| Seq ID | Name | Sequence | size |
| 20 | Fwd_screen_Are1 | TTTTTCTCGCCTTTGCGCTGACT | 1957 bp |
| 21 | Rev_screen_hyg | CGTCGCGGTGAGTTCAGGCTTTT | |
| 22 | Rev_screen_hyg | CGTCGCGGTGAGTTCAGGCTTTT | |
| 23 | Rev_screen_hyg | CGTCGCGGTGAGTTCAGGCTTTT | |
| 24 | Fwd_screen_hygDwn | CACTCGTCCGAGGGCAAAGGAA | 1667 bp |
| 25 | Rev_screen_areDwn | ACTGAAGAGGGGCCTAAGAAAG | 4637 bp |
| 26 | Fwd_screen_hygDwn | CACTCGTCCGAGGGCAAAGGAA | 1602 bp |
| 27 | Fwd_screen_hygDwn | CACTCGTCCGAGGGCAAAGGAA | 1642 bp |

### Example 3: Assessing Recombinant Protein Stability

To determine the presence of proteases produced by T. reesei with a deletion in Are1, in comparison with the parent microbial host cell, the stability of VHH-1 (which is a heavy chain variable domain of a heavy chain antibody or a functional fragment thereof) was evaluated in shake flasks with Vogel's medium with peptone or ammonium as nitrogen source prepared as defined in Example 8.

Approximately 5×10⁶ spores/mL of fresh conidia of the parent and modified T. reesei host cell were inoculated into 50 mL of Vogel's liquid medium (with peptone or ammonium as nitrogen source) in 250 ml shake flask in duplicate and incubated at 30° C. An uninoculated control was included in all experiments. After 48 h of growth, 500 μL of purified VHH-1 (28.61 mg/mL) was spiked into fermentation media and the addition of 1000 UL of 20% lactose inducer was started once a day.

During the induction process, 1 ml of homogeneous fermentation medium was taken daily from each flask to determine total soluble protein (TSP) by Bradford Protein assay kit (Thermo Scientific), according to the manufacturer's instructions.

The fermentation broth on day 2, day 4, day 6, day 8 and day 11 after cellulose induction was sampled and all the samples were separated by SDS-PAGE electrophoresis to visualize the degradation of VHH-1, taking 30 UL of fermentation broth, 7.5 μL sample buffer and 3.5 μL DTT, and denaturing the samples at 85° C. for 5 min. The samples were immediately transferred to ice before being loaded on SDS-PAGE gels (precast NuPAGE™ 4 to 12%, Bis-Tris, 1.0 mm, Mini Protein Gel, 12-well, Invitrogen).

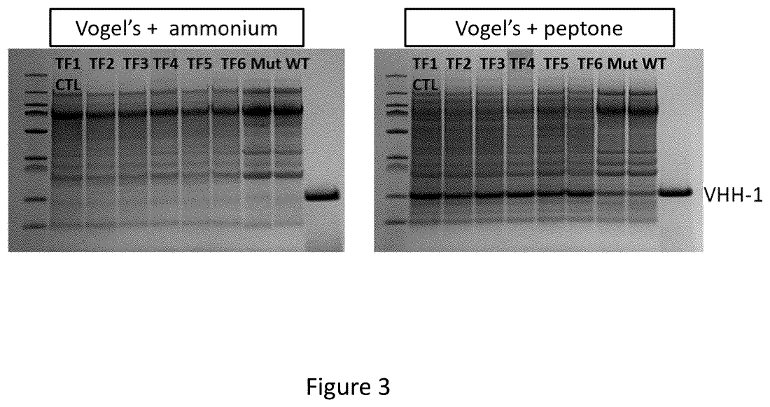

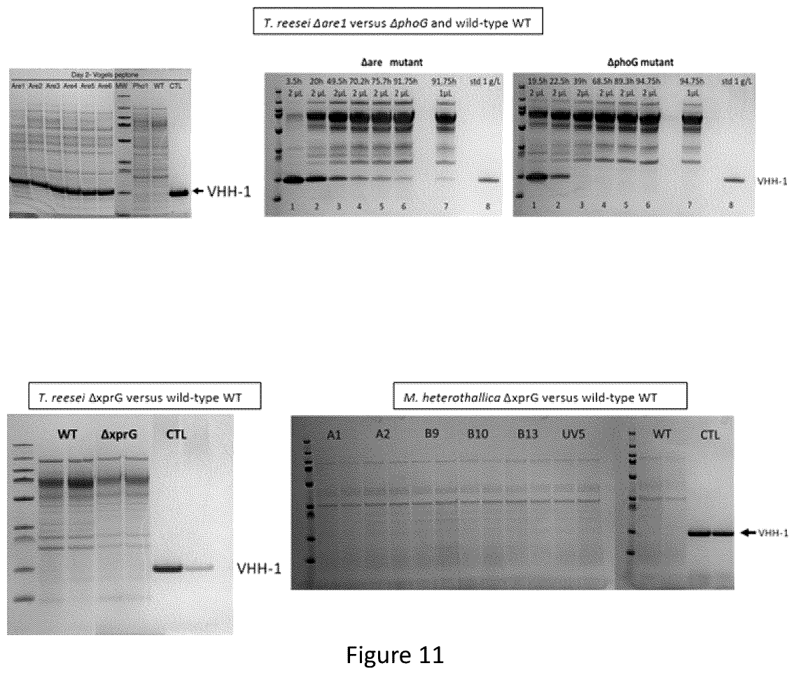

In FIG. 2 it is possible to observe the degradation process of the spiked VHH-1 during fermentation sampling for 11 days in Vogel's media containing peptone. The modified T. reesei cells showed lower degradation of VHH-1, being the VHH detectable up to 11 days post lactose induction as shown in FIG. 2. The concentration of spiked VHH-1 was visibly more abundant in Vogel's peptone culture media in all the modified T. reesei cells analyzed, which indicates that the deletion of the transcription factor Are1 has a suppressive effect on the secretion of proteases in T. reesei. Surprisingly, using Vogel's media with ammonium as a nitrogen source may lead to the rapid degradation of VHH-1 in all samples as opposed to Vogel's media with peptone as a nitrogen source as shown in FIG. 3. Notably, the deletion of alternative transcription factors that were suspected to have an effect on protease production did not lead to the desired effect. As shown in FIG. 11, deletion of the transcription factor PhoG and XprG in T. reesei and the deletion of XprG in M. heterothallica did not improve stability of a VHH-1 spiked in the respective fermentation broths. VHH-1 stability was assessed after at least 24h or up to 95 hours after spiking. It is noted that PhoG and XprG do not comprising the conserved sequence of SEQ ID NO: 31, which surprisingly indicates a correlation with the disruption of transcription factors comprising the conserved sequence of SEQ ID NO: 31 and the decrease in degradation of compounds of interest, in particular VHHs.

### Example 4: Assessing Cellulase Production

Since protein expression can be driven from the cellulase cbhl or cbh//promoters in some cases it could be important that production from these promoters is not impaired in the modified microbial host cell. This was evaluated by monitoring changes in cellulase production when compared to the parent microbial host cell. Therefore, in parallel, dilution samples of fermentation broth were analysed by pNP-cellobiohydrolase assay to determine the modified microbial host cell's cellulolytic ability (Coconi Linares et al., 2019).

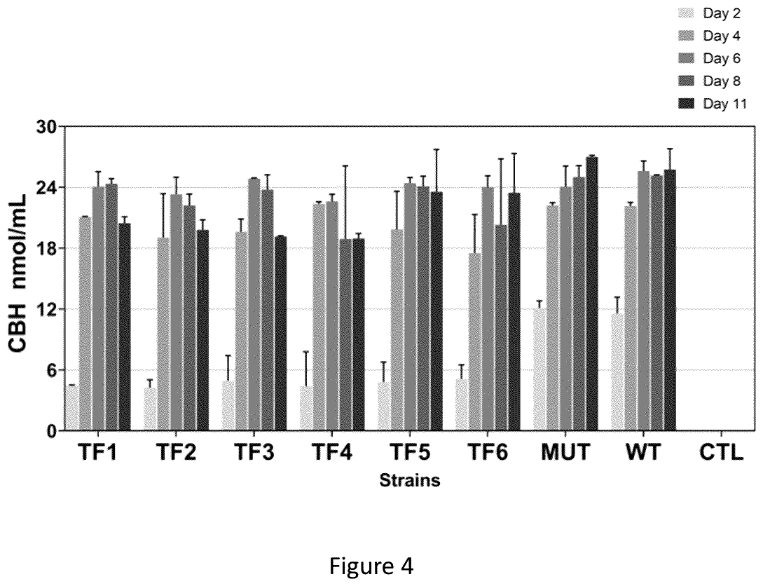

Surprisingly, in Vogel's medium with peptone as the nitrogen source the amount of cellulolytic activity did not greatly differ between modified T. reesei cells and the control samples as shown in FIG. 4.

### Example 5: Mass Spectrometry

To identify the abundance and repertoire of proteins secreted by the modified microbial host cell compared to the parent microbial host cell, the cells were cultured in fermenters using a fermentation culture medium such as described in Example 8, or in a defined medium (Trire) containing ammonium sulphate (NH4)₂SO₄ and peptone using either lactose or sophorose as inducers. Samples obtained from these fermentation cultures were TCA-precipitated, digested with trypsin, labelled, and analysed by liquid chromatography-tandem mass spectrometry (LC-MS/MS). With this analysis, it was possible to identify the proteases that are repressed or reduced in the modified microbial host cell in comparison to the parent microbial host cell.

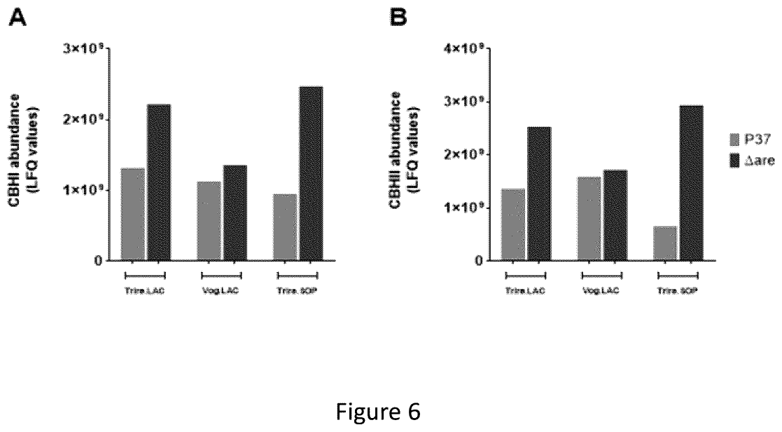

In more detail, total protein was TCA-precipitated from the supernatant broths, digested with trypsin, iTRAQ-labeled, and LC-MS/MS analyzed. MS raw files were imported into MaxQuant and proteins were identified and quantified using the MaxLFQ algorithm to compare the CBHI and CBHII protein abundances between the different samples. The LFQ (label-free quantitation) protein values were normalized to exclude some outliers to best represent the ratio changes of different samples. As shown in FIG. 6, the production of cellulases was increased in all the samples where the modified T. reesei strain was grown. The highest cellulase production was observed in Trire medium.

In this analysis, it was also observed that in the modified T. reesie cell, several proteases were less abundant compared to the quantity of the same proteases in the parent microbial host cell. Based on these results, it is concluded that Are1 plays a role in the proteolytic activity whilst maintaining and even improving the production of the main cellobiohydrolases.

### Example 6: Genomic Integration of a Recombinant Protein Expression Cassette

To generate a recombinant protein expression cassette, a codon-optimized version of VHH-1 gene and reference V_{HH} (refVHH) gene fused with the cellobiohydrolase I (CBHI) signal peptide coding sequence, and under the control of the cbhl or cbhll promoter sequences was synthesized. Alternatively, the catalytic domain fragment of cbh/was fused with the intact codon-optimized version of the selected recombinant gene, including the KexB protease cleavage site to release the recombinant protein and Cbhl carrier protein separately during during protein secretion. In addition, to ensure secretion and integration of the target proteins, the same expression cassettes mentioned above were readapted for their targeted integration in the cbh1 locus. The expression cassettes containing the cbhl or cbhll promoters with the target protein were flanked with 5′ and 3′ DNA homologous regions (˜1000 bp each) of cbh/locus, which results in the cbhl coding region replacement by the target gene. Both the VHH-1 and a reference VHH (refVHH) were introduced in the modified Trichoderma reesei strain and the parental Trichoderma reesei strain.

To construct a selection marker cassette, a fragment of about 1.5 kbp containing nptII/neo encoding neomycin phosphotransferase gene, as well as the the oliC promoter and the trpC terminator of Aspergillus nidulans (PoliC-hph-TtrpC) was obtained via gene synthesis. The enzyme neomycin phosphotransferase catalyses the hydrolysis of G418 antibiotic, thus conferring the ability to the microbial host cell to grow on that antibiotic. The co-transformation of nptII selection marker and recombinant protein expression cassette was performed as described in Example 2. After transformation, protoplasts were incubated at 28° C. for 4-6 days on selection plates containing 100 μg/mL of G418 on PDA plates. To confirm the integration of the expression cassettes, colony PCR was performed under standard PCR conditions with sequence-specific PCR primers. Both VHH-1 and a reference V_{HH} (refVHH) were introduced separately in the modified Trichoderma reesei strain and the parent Trichoderma reesei strain.

### Example 7: Recombinant Protein Expression

To compare the increase in efficiency of recombinant protein production, a modified microbial host cell expressing recombinant protein was compared to a parent microbial host cell expressing recombinant proteins.

The stable transformants were inoculated in production medium on shake flasks and incubated for several days. Then, the supernatants were collected and separated by SDS-PAGE electrophoresis to visualize the production of VHHs.

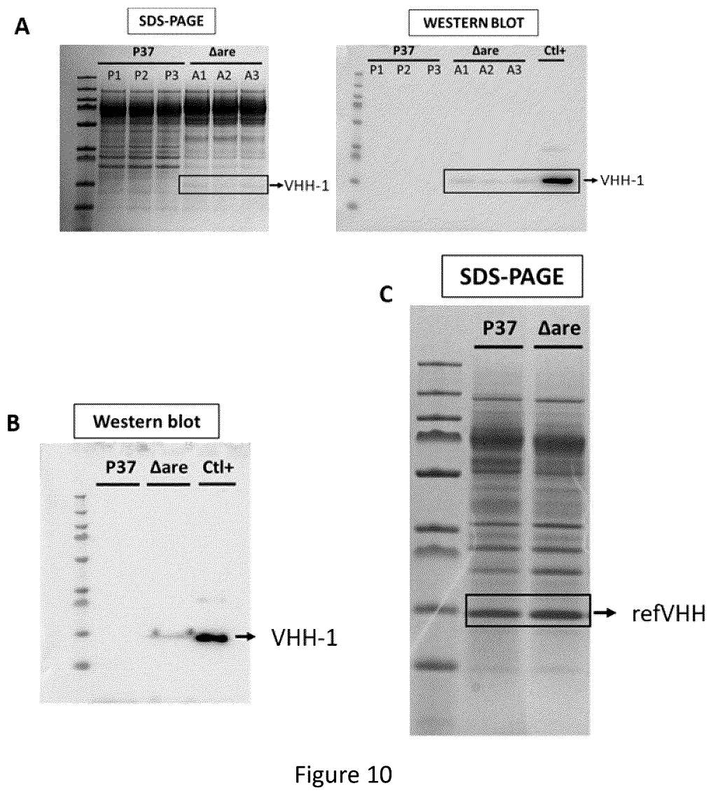

As shown in FIG. 10A., a significant expression of VHH-1 using CBHI catalytic domain as secretion carrier was observed in the modified microbial host transformants, in comparison to the parent microbial host transformants. Similar results were obtained with just the Cbhl signal sequence without Cbhl carrier (FIG. 10B). The production of VHH-1 using the two different types of expression strategies was particularly advantageous in modified microbial host transformants. To show the potential of modified microbial host strain to express other VHHs, a reference VHH (refVHH) was expressed under the cbh1 promoter without carrier in the parental strain and modified host strain (FIG. 10C). The production of the reference VHH was slightly increased as well in the modified host as can be judged by the intensity of the corresponding band on the SDS-PAGE gel.

### Example 8: General Culturing and Fermentation Broth Compositions

In some experiments the culturing or fermentation broth is composed of essentially the following ingredients:

TABLE 5
50 × VOGEL'S stock solution

| Vogel's 50x stock solution | Concentration (1 L) | |
|---|---|---|
| Na₃Citrate•2H₂O | 125 | g |
| KH₂PO₄ (anhydrous) | 250 | g |
| (NH₄)₂SO₄ | 100 | g |
| MgSO₄•7H₂O | 10 | g |
| CaCl₂•2H₂O | 5 | g |
| Vogel's Trace Element | 5 | mL |
| Solution | | |

| | |
|---|---|
| Biotin solution 0.1 mg | 2.5 mL/12.5 μL |
| or 20 mg/mL | |

Or alternatively:

| | | |
|---|---|---|
| Na₃Citrate•2H₂O | 125 | g |
| KH₂PO₄ (anhydrous) | 250 | g |
| Peptone | 50 | g |
| MgSO₄•7H₂O | 10 | g |
| CaCl₂•2H₂O | 5 | g |
| Vogel's Trace Element | 5 | mL |
| Solution | | |

| | |
|---|---|
| Biotin solution 0.1 mg | 2.5 mL/12.5 μL |
| or 20 mg/mL | |

TABLE 6
Vogel's trace element solution:

| Vogel's trace element solution | Concentration (1 L) |
|---|---|
| Citric acid | 50 g |
| ZnSO₄•7H₂O | 50 g |
| Fe(NH₄)₂SO₄•6H₂O | 10 g |
| CuSO₄•5H₂O | 2.5 g |
| MnSO₄•4H₂O | 0.5 g |
| H₃BO₃ | 0.5 g |
| Na₂MoO_{4•}2H₂O | 0.5 g |

TABLE 7
glucose concentration

| Glucose (50%) | Concentration |
|---|---|
| Glucose | 50% w/v |

TABLE 8
Final medium composition

| Medium composition | 500 mL |
|---|---|
| 50x Vogel's stock solution | 10 mL |
| Glucose (50% stock solution) | 10 mL |
| Yeast extract (80 g/L | 47 mL (0.75%) |
| autoclaved stock) | |

### Example 9: General Procedures for Performing a Fermentation

Fermenters are filled with medium with similar characteristics as described in Table 8 of Example 8, or in a defined medium (Trire) containing ammonium sulphate (NH4)₂SO₄ and peptone using either lactose or sophorose as inducers. Calibration of the Dissolved oxygen (DO) levels is performed at around 37° C., 400 rpm and 60 sL/h of aeration. The pH of the medium in the fermenter is adjusted to around 5 before being inoculated in the fermenter.

Fermenters are inoculated with around 0.5%-10% inoculum density in 1980 ml medium. Incubation at around 28° C.; 1200 rpm and 60 sL/h aeration. DO lower limit at 50%. DO cascade output set as 0-40% 1200-1400 rpm of stirrer, 40-100%, 100-200 tl/h of aeration. Antifoam is dissolved as 10× in water. Ammonium hydroxide 12.5% as base. Induction with for instance lactose 20% is generally initiated after a p02 spike. The feed rate is set at approximately 9 ml/h (4.5 ml/L.h).

### Example 10: Formulation

Recombinant proteins are produced in the appropriate microbial host cell and secreted into the fermentation media during fermentation. Recombinant proteins are then purified from media components and cell constituents by using common filtration and chromatographic techniques.

The resulting protein solution is diluted in a suitable buffer, such as phosphate buffered saline, to adjust the pH to about 7. Optionally a biocidal agent, such as sodium azide in a concentration of about 0.0001% to 0.1% and a non-ionic detergent, such as Tween20 in a concentration of about 0.0001% to 5%, is added to the buffered protein solution.

### Example 11: Decrease in Protease Activity of Modified T. reesei Host Cell

To investigate the effect of the composition of the culture media on the production of extracellular proteases of Δ are1 and RL-P37 parental strain, two culture media (defined medium (Trire) with ammonium sulphate (NH4)2SO4 and peptone or Vogel's minimal media), and two cellulolytic inducers (lactose and sophorose) were tested. For all experiments, fed-batch cultivation in 3 L fermenters were run at pH 4.8 and 28° C. for 6 days. The supernatant was separated after centrifugation and stored at −20° C. until the measurements of protease content. An estimate of the protease activity of the supernatant was made using a quantification assay available as the Pierce™ protease colorimetric assay kit (Thermo Scientific). Briefly, 10 μl culture superatant was diluted with assay buffer and duplicated in two different sets of wells to serve as blanks. Then, 100 μl succinylated casein solution to one set of microplate wells were added and 100 μl assay buffer to the other set, the samples were mixed and incubated for 20 min at 37° C. To measure the casein cleavage assay reaction 50 μl of TNBSA reagent was added to every well and the plate was incubated for 20 min at RT. The absorbance at 450 nm was measured for the whole plate. Control wells with supernatant without substrate were used as background controls. The nonspecific background signal was subtracted from specific protease quantification measurements.

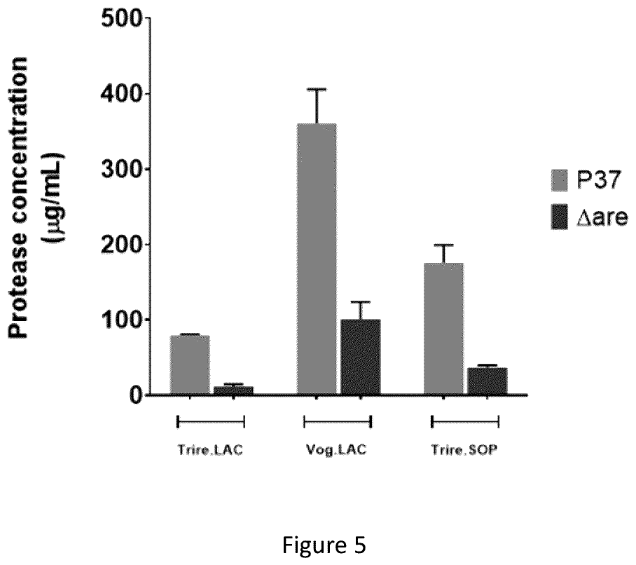

The decreases in protease concentration as estimated by the colorimetric assay kit were substantial in all the conditions tested using the modified T. reesei cell (Δ Are), while the samples with the parental T. reesei cell showed a significant increase in the amount of protease after 6 days of culture (FIG. 5). These findings were consistent with the reduced degradation of spiked V_{HH} using the modified T. reesei cells as compared to the parental T. reesei cells.

### Example 12: Additional Microbial Host Cell Proteins and Genes

The identified additional microbial host cell, specifically Myceliophthora heterothallica strain CBS 203.75, target polypeptide may be defined by peptide sequence (SEQ ID NO 28), genomic DNA sequence (SEQ ID NO: 29) and/or coding DNA sequence (SEQ ID NO: 30).

### Example 13: Generation of Genomic Modifications in Microbial Host Cells

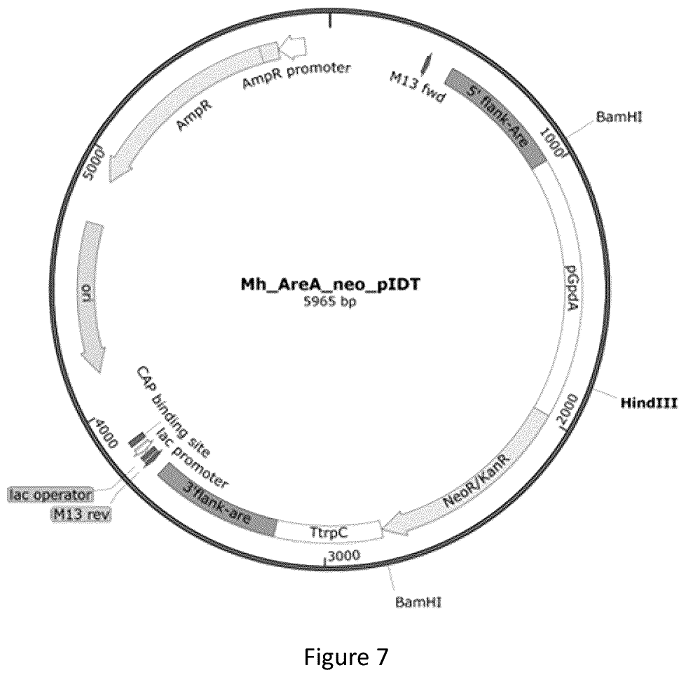

To delete the AreA gene from the genome of Myceliophthora heterothallica, the fragments necessary to generate the deletion cassette or DNA donor were synthesized. The 5′ and 3′ flanking fragments of AreA (500 bp/500 bp) were fused with a selection marker expression cassette containing the nptII gene (encoding neomycin phosphotransferase) codon-optimized, under the control of the gpdA promoter and the trpC terminator from Myceliophthora heterothallica and Aspergillus nidulans, respectively. The flanking fragments of AreA including the neomycin selection marker were cloned into cloning vector pIDT-amp (FIG. 7) and transformed to E. coli DH5a competent cells.

For confirming the correct generation of the AreA-neo donor, the plasmid was digested with HindIII and BamHI, followed by agarose electrophoresis to compare the inserted fragment lengths of the obtained clones. To obtain sufficient DNA for the Myceliophthora heterothallica transformation, a PCR was performed with the primers as described in Table 9 using the Are1-neo donor pIDT-amp plasmid as a template, resulting in the PCR DNA donor.

TABLE 9
Primers used for the donor amplification

| SEQ ID NO | Name | Sequence | Product size |
|---|---|---|---|
| 39 | dAreMh_500neo_fwd | GGGCAATCACCCGCTTTTTCC | 3213 bp |
| 40 | dAreMh_500neo_rev | CCCGAACCAAAAGCTCTGAT | |

Transformation of microbial host cells with the target DNA and was achieved by a standard poly-ethylene glycol (PEG) mediated transformation method as described previously (dos Santos Gomes et al. 2019). Successful transformants were selected on minimal medium containing AspA+N, 2 mM MgSO4, 0.1% trace elements, 0.1% casamino acids, 670 mM sucrose, 1% D-glucose and 1.5% agar with 100 mg/mL G-418 as the selective agent. The plates were incubated for 4 days until colonies could be picked to secondary selection plates. The neomycin-resistant colonies and parent microbial host cells were grown in YPD liquid medium (10 g/L yeast extract, 20 g/L peptone, and 20 g/L glucose). Genomic DNA was extracted from colonies using Phire Plant Direct PCR Kit (Thermo Fisher Scientific) according to the manufacturer's instructions. The resulting genomic DNA sample was diluted into 10 μl water, mycelium was removed by centrifugation, and the supernatant was used as a template in subsequent PCR.

Oligonucleotides were designed outside of the target locus to neomycin selection marker gene to identify the possible integration of the donor cassette into the deletion region (Table 10). The expected size of the neomycin expression cassette was integrated into the AreA locus gene, observing 3589 bp of the amplicon size in the transformants examined, in contrast, the intact AreA gene in the parental strain was observed around 4800 bp. In conclusion, the AreA gene was successfully deleted from the Myceliophthora heterothallica strain.

TABLE 10
Primers used for screening of donor cassette integration into the deletion region

| SEQ ID NO | Name | Sequence | Product size |
|---|---|---|---|
| 41 | Mh_are_screen_F | CCACGGGAATCTCCACCGTTCTCA | 3589 bp |
| 42 | Mh_are_screen_R | TACAGTAGACAGCGAGCAACCACAG | |

### Example 14: Assessing Recombinant Protein Stability

To determine the presence of proteases produced by M. heterothallica with a deletion in AreA, in comparison with the parent microbial host cell, the stability of VHH-1 (which is a heavy chain variable domain of a heavy chain antibody or a functional fragment thereof) was evaluated in shake flasks with minimal medium with a combination of ammonium and peptone as nitrogen source.

Approximately 1×105 spores/ml of fresh conidia of the parental and modified M. heterothallica strains were inoculated into 50 ml YPD medium in 250 ml shake flasks and incubated overnight at 45° C. and 150 rpm to obtain enough biomass. After 24 h of growth, 10 ml of the mycelium was added to 40 ml of minimal culture medium containing 25 ml/L AspA+NH4, 1% peptone, 2 mM MgSO4, 0.1% trace elements, 0.1% casamino acids, and 4 μg/L biotin. In each shake flask, 300 UL of purified VHH-1 (10 mg/mL) was spiked into fermentation media, and supplemented with 0.5 ml lactose (20%) and 0.5 ml Avicel (25%) as inducers were added. The shake flasks were incubated at 37° C. and 150 rpm for 4 days approximately.

During the induction process, 1 ml of homogeneous fermentation medium was taken daily from each flask to determine total protease concentration and VHH-1 stability. To investigate the effect of deletion of

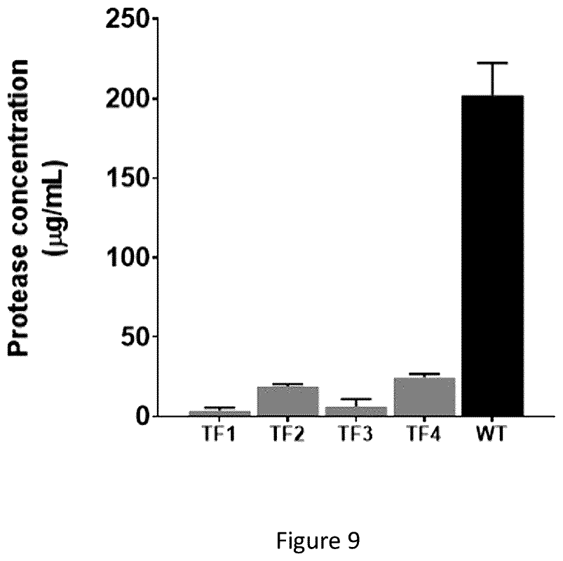

AreA on the production of extracellular proteases, four ΔareA deleted strains (TF1 to TF4) and the M. heterothallica wild type (WT) were tested by Pierce™ protease colorimetric assay kit (Thermo Scientific) according to the manufacturer's instructions. As shown in FIG. 9, the protease concentration in the wild-type strain was over 8 times higher in comparison to the observed in the deleted areA transformants. These results confirm that the proteolytic activity was affected by the deletion of areA transcription factor.

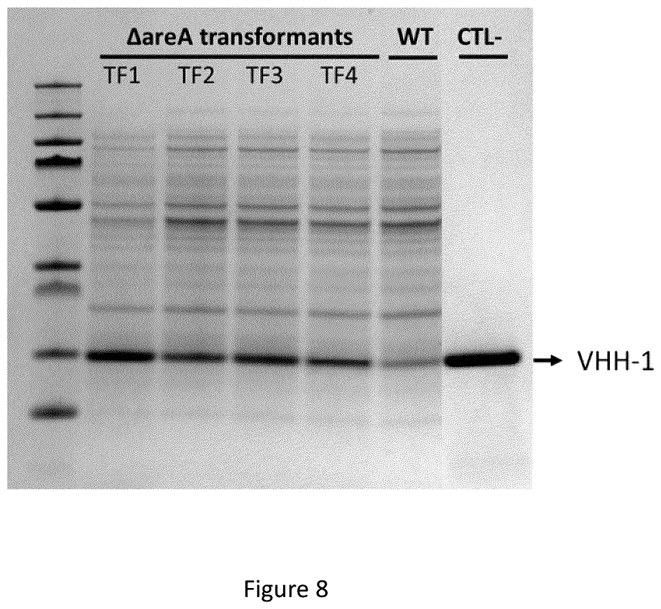

Furthermore, all the samples were separated by SDS-PAGE electrophoresis to visualize the degradation of the spiked VHH-1, taking 30 μL of fermentation broth, 7.5 μL sample buffer, and 3.5 μL DTT, and denaturing the samples at 85° C. for 5 min. The samples were immediately transferred to ice before being loaded on SDS-PAGE gels (precast NuPAGE™ 4 to 12%, Bis-Tris, 1.0 mm, Mini Protein Gel, 12-well, Invitrogen). In FIG. 8 it is possible to observe the degradation process of the spiked VHH-1 during fermentation sampling for 3 days in fermentation media. The modified M. heterothallica cells showed lower degradation of VHH-1, being the V_{HH} detectable up to 3 days post-lactose-avicel induction. The concentration of spiked VHH-1 was visibly more abundant in all the modified M. heterothallica samples analyzed, which indicates that the deletion of the transcription factor AreA has a suppressive effect on the expression of proteases in M. heterothallica.

### REFERENCES

Coconi Linares, N., Di Falco, M., Benoit-Gelber, I., Gruben, B. S., Peng, M., Tsang, A., Mäkelä, M. R., de Vries, R. P., 2019. The presence of trace components significantly broadens the molecular response of Aspergillus niger to guar gum. N. Biotechnol. 51, 57-66. https://doi.org/10.1016/j.nbt.2019.02.005
Penttila, M., Nevalainen, H., Ratto, M., Salminen, E., Knowles, J., 1987. A versatile transformation system for the cellulolytic filamentous fungus Trichoderma reesei. Gene 61, 155-64.
Vanegas, K. G., Jarczynska, Z. D., Strucko, T., Mortensen, U. H., 2019. Cpf1 enables fast and efficient genome editing in Aspergilli. Fungal Biol. Biotechnol. 6, 1-10. https://doi.org/10.1186/s40694-019-0069-6
Vogel, H., 1956. A convenient growth medium for Neurospora. Microbiol. Genet. Bull. 13, 42-43
dos Santos Gomes, A. C., Falkoski, D., Battaglia, E., Peng, M., Nicolau de Almeida, M., Coconi Linares, N., Meijnen, J.-P., Visser, J., de Vries, R. P., 2019. Myceliophthora thermophila Xyr1 is predominantly involved in xylan degradation and xylose catabolismbiotechnol. Biofuels 12, 1-18. https://doi org/10.1186/13068-019.1556.y

### Embodiments

The invention includes at least the following numbered embodiments:
1. A microbial host cell which is characterized by:
   a. having been modified and where this modification affects the production, stability and/or function of at least one polypeptide; and
   b. having a modulation in protease activity if compared with a parent microbial host cell which has not been modified and measured under the same conditions.
2. The microbial host cell of embodiment 1, wherein the at least one polypeptide comprises a sequence having at least about 95% sequence identity to SEQ ID NO: 31.
3. The microbial host cell of embodiment 1, wherein the at least one polypeptide comprises the sequence of SEQ ID NO: 31.
4. The microbial host cell of embodiment 1, wherein the at least one polypeptide comprises a sequence having at least about 95% sequence identity with the amino acids 685-735 of SEQ ID NO: 1, at least about 95% sequence identity with the amino acids 753-803 of SEQ ID NO: 28, at least 95% sequence identity with the amino acids 749-799 of SEQ ID NO: 33. at least about 95% sequence identity with the amino acids 670-720 of SEQ ID NO: 36, at least about 95% sequence identity with the amino acids 679-729 of SEQ ID NO: 58 or at least about 95% sequence identity with the amino acids 749-799 of SEQ ID NO: 59.
5. The microbial host cell of embodiment 1, wherein the at least one polypeptide comprises a sequence comprising amino acids 685-735 of SEQ ID NO: 1, amino acids 753-803 of SEQ ID NO: 28, amino acids 749-799 of SEQ ID NO: 33, amino acids 670-720 of SEQ ID NO: 36, amino acids 679-729 of SEQ ID NO: 58 or amino acids 749-799 of SEQ ID NO: 59.
6. The microbial host cell of any preceding embodiment, wherein the at least one polypeptide comprises a sequence having at least about 35% identity to a sequence selected from the group consisting of SEQ ID NOs: 1, 28, 33, 36, 58 and 59.
7. The microbial host cell of any preceding embodiment, wherein the at least one polypeptide comprises a sequence having at least about 40% identity to a sequence selected from the group consisting of SEQ ID NOs: 1, 28, 33, 36, 58 and 59.
8. The microbial host cell of any preceding embodiment, wherein the at least one polypeptide comprises a sequence having at least about 90% identity to a sequence selected from the group consisting of SEQ ID NOs: 1, 28, 33, 36, 58 and 59.
9. The microbial host cell of any preceding embodiment, wherein the at least one polypeptide comprises a sequence having at least about 95% identity to a sequence selected from the group consisting of SEQ ID NOs: 1, 28, 33, 36, 58 and 59.
10. The microbial host cell of any preceding embodiment, therein the at least one polypeptide comprises a sequence having at least about 95% identity to the sequence of SEQ ID NO: 31 and wherein the polypeptide has at least about 35% identity to a sequence selected from the group consisting of SEQ ID NOs: 1, 28, 33, 36, 58 and 59.
11. The microbial host cell of any preceding embodiment, therein the at least one polypeptide comprises a sequence having at least about 95% identity to the sequence of SEQ ID NO: 31 and wherein the polypeptide has at least about 40% identity to a sequence selected from the group consisting of SEQ ID NOs: 1, 28, 33, 36, 58 and 59.
12. The microbial host cell of any preceding embodiment, therein the at least one polypeptide comprises a sequence having at least about 95% identity to the sequence of SEQ ID NO: 31 and wherein the polypeptide has at least about 90% identity to a sequence selected from the group consisting of SEQ ID NOs: 1, 28, 33, 36, 58 and 59.
13. The microbial host cell of any preceding embodiment, therein the at least one polypeptide comprises a sequence having at least about 95% identity to the sequence of SEQ ID NO: 31 and wherein the polypeptide has at least about 95% identity to a sequence selected from the group consisting of SEQ ID NOs: 1, 28, 33, 36, 58 and 59.
14. The microbial host cell of any one of embodiments 6 to 13, wherein:
   a. when the sequences of the at least one polypeptide is aligned with the sequence of SEQ ID NO: 1, at least about 50%, at least about 60%, at least about 70%, at least about 80% or at least about 90% of the variations between the at least one polypeptide comprising a sequence having at least about 35% identity, at least about 40% identity, at least about 90% identity or at least about 95% identity to the sequence of SEQ ID NO: 1 and the sequence of SEQ ID NO: 1 are conservative amino acid substitutions;
   b. when the sequences of the at least one polypeptide is aligned with the sequence of SEQ ID NO: 28, at least about 50%, at least about 60%, at least about 70%, at least about 80% or at least about 90% of the variations between the at least one polypeptide comprising a sequence having at least 35% identity, at least about 40% identity, at least about 90% identity or at least about 95% identity to the sequence of SEQ ID NO: 28 and the sequence of SEQ ID NO: 28 are conservative amino acid substitutions;
   c. when the sequences of the at least one polypeptide is aligned with the sequence of SEQ ID NO: 33, at least about 50%, at least about 60%, at least about 70%, at least about 80% or at least about 90% of the variations between the at least one polypeptide comprising a sequence having at least 35% identity, at least about 40% identity, at least about 90% identity or at least about 95% identity to the sequence of SEQ ID NO: 33 and the sequence of SEQ ID NO: 33 are conservative amino acid substitutions;
   d. when the sequences of the at least one polypeptide is aligned with the sequence of SEQ ID NO: 36, at least about 50%, at least about 60%, at least about 70%, at least about 80% or at least about 90% of the variations between the at least one polypeptide comprising a sequence having at least 35% identity, at least about 40% identity, at least about 90% identity or at least about 95% identity to the sequence of SEQ ID NO: 36 and the sequence of SEQ ID NO: 36 are conservative amino acid substitutions;
   e. when the sequences of the at least one polypeptide is aligned with the sequence of SEQ ID NO: 58, at least about 50%, at least about 60%, at least about 70%, at least about 80% or at least about 90% of the variations between the at least one polypeptide comprising a sequence having at least 35% identity, at least about 40% identity, at least about 90% identity or at least about 95% identity to the sequence of SEQ ID NO: 58 and the sequence of SEQ ID NO: 58 are conservative amino acid substitutions or
   f. when the sequences of the at least one polypeptide is aligned with the sequence of SEQ ID NO: 59, at least about 50%, at least about 60%, at least about 70%, at least about 80% or at least about 90% of the variations between the at least one polypeptide comprising a sequence having at least 35% identity, at least about 40% identity, at least about 90% identity or at least about 95% identity to the sequence of SEQ ID NO: 59 and the sequence of SEQ ID NO: 59 are conservative amino acid substitutions.
15. The microbial host cell of embodiment 14, wherein conservative amino acid substitutions are defined as:
   a. the substitution of any glycine, alanine, valine, leucine or isoleucine residues in the reference sequence with another amino acid selected from glycine, alanine, valine, leucine and isoleucine;
   b. the substitution of any serine, cysteine, threonine or methionine residues in the reference sequence with another amino acid selected from serine, cysteine, threonine and methionine;
   c. the substitution of any phenylalanine, tyrosine or tryptophan residues in the reference sequence with another amino acid selected from phenylalanine, tyrosine and tryptophan;
   d. the substitution of any histidine, lysine or arginine residues in the reference sequence with another amino acid selected from histidine, lysine or arginine; and
   e. the substitution of any aspartate, glutamate, asparagine or glutamine residues in the reference sequence with another amino acid selected from aspartate, glutamate, asparagine and glutamine.
16. The microbial host cell of any preceding embodiment, wherein the at least one polypeptide comprises a GATA-type zinc finger domain.
17. The microbial host cell of any preceding embodiment, wherein the GATA-type zinc finger domain comprises a sequence having at least about 95% identity to SEQ ID NO: 31.
18. The microbial host cell of any preceding embodiment, wherein the GATA-type zinc finger domain comprising the sequence of SEQ ID NO: 31.
19. The microbial host cell of any preceding embodiment, wherein the polypeptide is fungal polypeptide.
20. The microbial host cell of any preceding embodiment, wherein the polypeptide is a polypeptide of a filamentous fungi.
21. The microbial host cell of any preceding embodiment, wherein the polypeptide is Are1 or AreA or an ortholog of Are1 or AreA.
22. The microbial host cell of any preceding embodiment wherein the at least one polypeptide comprises a sequence according to SEQ ID NO: 1 or a polypeptide at least about 80%, at least about 85%, at least about 90%, at least about 95%, or at least about 98% identical thereto, or an ortholog thereof.
23. The microbial host cell of any one of embodiments 1 to 21, wherein the at least one polypeptide comprises a sequence according to SEQ ID NO: 28 or a polypeptide at least about 80%, at least about 85%, at least about 90%, at least about 95%, or at least about 98% identical thereto, or an ortholog thereof.
24. The microbial host cell of any one of embodiments 1 to 21, wherein the at least one polypeptide comprises a sequence according to SEQ ID NO: 33 or a polypeptide at least about 80%, at least about 85%, at least about 90%, at least about 95%, or at least about 98% identical thereto, or an ortholog thereof.
25. The microbial host cell of any one of embodiments 1 to 21, wherein the at least one polypeptide comprises a sequence according to SEQ ID NO: 36 or a polypeptide at least about 80%, at least about 85%, at least about 90%, at least about 95%, or at least about 98% identical thereto, or an ortholog thereof.
26. The microbial host cell of any one of embodiments 1 to 21, wherein the at least one polypeptide comprises a sequence according to SEQ ID NO: 58 or a polypeptide at least about 80%, at least about 85%, at least about 90%, at least about 95%, or at least about 98% identical thereto, or an ortholog thereof.
27. The microbial host cell of any one of embodiments 1 to 21, wherein the at least one polypeptide comprises a sequence according to SEQ ID NO: 59 or a polypeptide at least about 80%, at least about 85%, at least about 90%, at least about 95%, or at least about 98% identical thereto, or an ortholog thereof.
28. The microbial host cell of any preceding embodiment, wherein the at least one polypeptide controls the expression of one or more proteases.
29. The microbial host cell of any preceding embodiment, wherein the at least one polypeptide interrupts, represses or halts the expression of one or more proteases.
30. The microbial host cell of any one of embodiments 1 to 28, wherein the at least one polypeptide causes, initiates or promotes the expression of one or more proteases
31. The microbial host cell of any preceding embodiment, wherein the modulation in protease activity is a reduction or deficiency in protease activity.
32. The microbial host cell of any preceding embodiment, wherein the modification adversely affects the production, stability and/or function of the at least one polypeptide.
33. The microbial host cell of any preceding embodiment, wherein the at least one polypeptide causes, initiates or promotes the expression of one or more proteases, the modification adversely affects the production, stability and/or function of the at least one polypeptide, and the modulation in protease activity is a reduction or deficiency in protease activity.
34. The microbial host cell of any one of embodiments 1 to 31, wherein the modification positively affects the production, stability and/or function of the at least one polypeptide.
35. The microbial host cell of any one of embodiments 1 to 31 or embodiment 34, wherein the at least one polypeptide interrupts, represses or halts the expression of one or more proteases, the modification positively affects the production, stability and/or function of the at least one polypeptide, and the modulation in protease activity is a reduction or deficiency in protease activity.
36. The microbial host cell of any preceding embodiment wherein the at least one polypeptide is a regulator of transcription.
37. The microbial host cell of embodiment 36, wherein the at least one polypeptide is a promoter of transcription or a repressor of transcription.
38. The microbial host cell embodiment 37, wherein the at least one polypeptide is a promoter of transcription and has been modified to reduce its production, stability and/or function, and the modulation in protease activity is a reduction or deficiency in protease activity.
39. The microbial host cell of embodiment 38, wherein the promoter of transcription has been modified to decrease the function of one or more DNA binding domains in the promoter of transcription that control the expression of one or more proteases.
40. The microbial host cell of embodiment 39, wherein the at least one polypeptide is a repressor of transcription and has been modified to positively affect its production, stability and/or function, and the modulation in protease activity is a reduction or deficiency in protease activity.
41. The microbial host cell of embodiment 40, wherein the at least one polypeptide is a repressor of transcription and has been modified to increase the function of one or more DNA binding domains in the promoter of transcription that control the expression of one or more proteases.
42. The microbial host cell of any one of embodiments 36 to 41, wherein any DNA binding domains in the regulator of transcription that control the expression of one or more cellulases have not been modified.
43. The microbial host cell of any preceding embodiment wherein the at least one polypeptide is coded for by a genomic nucleotide sequence comprising a sequence selected from the group consisting of SEQ ID NOS: 2, 29, 34 and 37 or a nucleotide sequence at least about 80%, at least about 85%, at least about 90%, at least about 95%, or at least about 98% identical thereto.
44. The microbial host cell of any preceding embodiment, wherein the at least one polypeptide is coded for by a nucleotide sequence comprising a sequence selected from the group consisting of SEQ ID NOS: 3, 30, 35 and 38 or a nucleotide sequence at least about 80%, at least about 85%, at least about 90%, at least about 95%, or at least about 98% identical thereto.
45. The microbial host cell of any preceding embodiment, wherein the polypeptide is an ortholog of a polypeptide defined in any one of embodiments 2 to 44.
46. The microbial host cell of embodiment 45, wherein the ortholog is an ortholog from a Trichoderma spp., a Myceliophthora spp., an Aspergillus spp., a Penicillium spp. or a Fusarium spp.
47. The microbial host cell of embodiment 46, wherein the ortholog is an ortholog from Trichoderma spp
48. The microbial host cell of embodiment 46, wherein the ortholog is an ortholog from Myceliophthora spp.
49. The microbial host cell of embodiment 46, wherein the ortholog is an ortholog from Aspergillus spp
50. The microbial host cell of any one of embodiments 45 to 49, wherein the ortholog performs the same function as the polypeptide.
51. The microbial host cell of any one of embodiments 45 to 50, wherein the ortholog is from the same genus of microbial host cell.
52. The microbial host cell of any one of embodiments 45 to 51, wherein the ortholog is from the same species of microbial host cell.
53. The microbial host of any one of embodiments 45 to 52, wherein the ortholog comprises at least about 35%, at least about 40%, at least about 45%, at least about 50%, at least about 60%, at least about 70%, at least about 80%, at least about 90%, at least about 95%, at least about 96%, at least about 97%, at least about 98% or at least about 99% sequence identity to a sequence selected from the group consisting of SEQ ID NOs: 1, 28, 33, 36, 58 and 59.
54. The microbial host of any one of embodiments 45 to 53, wherein the ortholog comprises a sequence having at least 95% identity to SEQ ID NO: 31.
55. The microbial host of any one of embodiments 45 to 54, wherein the ortholog comprises the sequence of SEQ ID NO: 31.
56. The microbial host cell of any preceding embodiment, wherein the microbial host cell has been further modified to affect the production, stability and/or function of one or more additional polypeptides.
57. The microbial host cell of embodiment 56, wherein the one or more additional polypeptides are one or more proteases.
58. The microbial host cell according to any preceding embodiment wherein the modification is a genetic modification.
59. The microbial host cell according to any preceding embodiment wherein the modification is a partial or full deletion, a truncation, a nucleotide insertion and/or a nucleotide substitution.
60. The microbial host cell according to embodiment 59, wherein the modification is a partial or full deletion.
61. The microbial host cell according to any preceding embodiment, wherein the microbial host cell or a fermentation broth or cell culture medium containing said modified microbial host cell has at least about 40% less protease activity if compared with the intracellular environment of the parent microbial host cell which has not been modified or a fermentation broth or cell culture medium containing said parent microbial host cell which has not been modified and measured under the same conditions.
62. The microbial host cell according to any preceding embodiment, wherein the modulation in protease activity is a reduction in intracellular protease activity.
63. The microbial host cell according to any preceding embodiment, wherein the modulation in protease activity is a reduction in extracellular protease activity.
64. The microbial host cell according to any preceding embodiment which is a eukaryotic cell.
65. The microbial host cell according to embodiment 64, wherein the eukaryotic cell is a fungal cell.
66. The microbial host cell according to embodiment 65, wherein the eukaryotic cell is a filamentous fungal host cell.
67. The microbial host cell according to any preceding embodiment, wherein the microbial host cell is a cell of a filamentous fungus selected from the group consisting of Aspergillus, Acremonium, Myceliophthora, Thielavia Chrysosporium, Penicillium, Talaromyces, Rasamsonia, Fusarium or Trichoderma, preferably a species of Aspergillus niger, A. nidulans, Aspergillus awamori, Aspergillus foetidus, Aspergillus sojae, Aspergillus fumigatus, Aspergillus oryzae, Acremonium alabamense, Myceliophthora thermophila, Myceliophthora heterothallica, Thermothelomyces heterothallica, Thermothelomyces thermophilus, Thielavia terrestris, Chrysosporium lucknowense, Fusarium oxysporum, Rasamsonia emersonii, Talaromyces emersonï, Trichoderma reesei, Penicillium chrysogenum, Penicillium oxalicum and Neurospora crassa.
68. The microbial host cell according to embodiment 67 which is Trichoderma reesei.
69. The microbial host cell according to embodiment 67, wherein the microbial host cell is a filamentous fungus selected from the group consisting of Trichoderma reesei QM6a, Trichoderma reesei Rut-C30, Trichoderma reesei RL-P37 and Trichoderma reesei MCG-80.
70. The microbial host cell according to embodiment 67 which is Myceliophthora heterothallica.
71. The microbial host cell according to embodiment 67, wherein the microbial host cell is a filamentous fungus selected from the group consisting of Myceliophthora heterothallica CBS 131.65, Myceliophthora heterothallica CBS 203.75, Myceliophthora heterothallica CBS 202.75, Myceliophthora heterothallica CBS 375.69 and Myceliophthora heterothallica CBS 663.74.
72. The microbial host cell according to embodiment 67 which is Myceliophthora thermophila.
73. The microbial host cell according to embodiment 67, wherein the microbial host cell is a filamentous fungus selected from the group consisting of Myceliophthora thermophila ATCC42464, Myceliophthora thermophila ATCC26915, Myceliophthora thermophila ATCC48104, Myceliophthora thermophila ATCC34628, Thermothelomyces heterothallica C1 and Thermothelomyces thermophilus M77
74. The microbial host cell according to embodiment 67 which is Aspergillus nidulans.
75. The microbial host cell according to embodiment 67, wherein the microbial host cell is a filamentous fungus selected from the group consisting of Aspergillus nidulans FGSC A4 (Glasgow wild-type), Aspergillus nidulans GR5 (FGSC A773), Aspergillus nidulans TN02A3 (FGSC A1149), Aspergillus nidulans TNO2A25, (FGSC A1147), Aspergillus nidulans ATCC 38163 and Aspergillus nidulans ATCC 10074.
76. The microbial host cell of any preceding embodiment, wherein the microbial host cell further comprises at least one polynucleotide coding for a compound of interest.
77. The microbial host cell according to embodiment 76 wherein the at least one polynucleotide coding for the compound of interest is operably linked to a promoter, optionally to an inducible promoter.
78. The microbial host cell of any one of embodiments 76 to 77, wherein the compound of interest is an antibody or a functional fragment thereof, a carbohydrate binding domain, a heavy chain antibody or a functional fragment thereof, a single domain antibody, a heavy chain variable domain of an antibody or a functional fragment thereof, a heavy chain variable domain of a heavy chain antibody or a functional fragment thereof (VHH), a variable domain of camelid heavy chain antibody or a functional fragment thereof, a variable domain of a new antigen receptor (vNAR), a variable domain of shark new antigen receptor or a functional fragment thereof, a minibody, a nanobody, a nanoantibody, an affibody, an alphabody, a designed ankyrin-repeat domain, an anticalins, a knottins or an engineered CH2 domain
79. The microbial host cell of embodiment 78, wherein the compound of interest is an antibody or a functional fragment thereof.
80. The microbial host cell of embodiment 79, wherein the antibody or functional fragment thereof of is variable domain of camelid heavy chain antibody (VHH).
81. The microbial host cell of embodiment 79, wherein the VHH is a VHH comprising:
   a. a CDR1 comprising or consisting of a sequence selected from the group consisting of SEQ ID NOs 45, 49 and 53;
   b. a CDR2 comprising or consisting of a sequence selected from the group consisting of SEQ ID NOs: 46, 50 and 54; and
   c. a CDR3 comprising or consisting of a sequence selected from the group consisting of SEQ ID NOs: 47, 51 and 55.
82. The microbial host cell of embodiment 79, wherein the VHH is a VHH comprising:
   a. a CDR1 comprising or consisting of the sequence of SEQ ID NO: 45, a CDR2 comprising or consisting of the sequence of SEQ ID NO: 46 and a CDR3 comprising or consisting of the sequence of SEQ ID NO: 47;
   b. a CDR1 comprising or consisting of the sequence of SEQ ID NO: 49, a CDR2 comprising or consisting of the sequence of SEQ ID NO: 50 and a CDR3 comprising or consisting of the sequence of SEQ ID NO: 51 or
   c. a CDR1 comprising or consisting of the sequence of SEQ ID NO: 53, a CDR2 comprising or consisting of the sequence of SEQ ID NO: 54 and a CDR3 comprising or consisting of the sequence of SEQ ID NO: 55.
83. The microbial host cell of embodiment 79, wherein the VHH is a VHH comprising or consisting of a sequence selected from the group consisting of SEQ ID NOs: 43, 44, 48, 52, 56 and 57.
84. The microbial host cell of embodiment 79, wherein the VHH is a VHH comprising or consisting of SEQ ID NO: 43.
85. The microbial host cell of embodiment 79, wherein the VHH is a VHH comprising or consisting of SEQ ID NO: 44.
86. The microbial host cell of any one of embodiments 76 to 85, wherein the compound of interest is fused to a carrier peptide.
87. The microbial host cell of any one of embodiments 76 to 86, wherein the polynucleotide coding for a compound of interest encodes, in a 5′ to 3′ order, a carrier peptide, a proteolytic cleavage side, and the compound of interest.
88. A method of producing a microbial host cell according to any preceding embodiment comprising the steps of:
   a. providing a parent microbial host cell; and
   b. modifying the parent microbial host cell, wherein the modification affects the production, stability and/or function of the at least one polypeptide.
89. The method of embodiment 88, wherein the step of modifying the parent microbial host cell comprises targeting the at least one polypeptide, its corresponding chromosomal gene and/or its corresponding mRNA by anti-sense techniques, RNAi techniques, CRISPR techniques, a small molecule inhibitor, an antibody, an antibody fragment or a combination thereof.
90. The method of embodiment 88 or embodiment 89, wherein the method further comprises inserting a polynucleotide coding for a compound of interest into the microbial host cell.
91. A method for the production of a compound of interest comprising:
   a. providing a microbial host cell according to any one of embodiments 1 to 87 or produced by a method according to any one of embodiments 88 to 90, wherein the microbial host cell is capable of expressing the compound of interest;
   b. culturing said microbial host cell under conditions conducive to the expression of a compound of interest; and
   c. optionally isolating compound of interest from the culture medium.
92. The method according to embodiment 91 wherein the compound of interest is a pharmacologically or agrochemically active polypeptide.
93. The method according any one of embodiments 91 to 92, wherein the yield of the compound of interest is increased by at least about 1%, at least about 2%, at least about 3%, at least about 4%, at least about 5%, at least about 6%, at least about 7%, at least about 8%, at least about 9%, at least about 10%, at least about 20%, at least about 30%, at least about 40%, at least about 50%, at least about 60%, at least about 70%, at least about 80%, at least about 90%, at least about 100%, at least about 110%, at least about 120%, at least about 130%, at least about 140%, at least about 150%, at least about 160%, at least about 170%, at least about 180%, at least about 190%, at least about 200%, at least about 210%, at least about 220%, at least about 230%, at least about 240%, at least about 250%, at least about 260%, at least about 270%, at least about 280%, at least about 290% or at least about 300%, at least about 500%, at least about 1000% or at least about 1500% when compared to a parent host cell and measured under the same conditions.
94. The method according to embodiment 93, wherein the field of the compound of interest is increased by at least about 100% when compared to a parent host cell measured under the same conditions.
95. The method of any one of embodiments 91 to 94, wherein the method further comprises a step of formulating the compound of interest into a pharmaceutical composition or an agrochemical composition.
96. The method of embodiment 95, wherein the step of formulation the compound of interest comprises formulation the compound of interest with one or more pharmaceutically acceptable excipients, or one or more agrochemically acceptable excipients.
97. The method of any one of embodiments 91 to 96, wherein the compound of interest is a VHH.
98. The method of embodiment 97, wherein the VHH is a VHH as defined in any one of embodiments 81 to 87,
99. Use of a modified microbial host cell for the production of a compound of interest, wherein the microbial host cell is characterized by (a) having been modified and where this modification affects the production, stability and/or function of at least one polypeptide; (b) having a reduction or deficiency in protease activity if compared with a parent microbial host cell which has not been modified and is measured under the same conditions; and (c) comprising at least one polynucleotide coding for the compound of interest.
100. The use of a microbial host cell of embodiment 99, wherein the microbial host cell is a microbial host cell according to any one of embodiments 1 to 87.
101.A kit comprising:
   a. a microbial cell; and
   b. a vector for homologous recombination, for example for effecting a full or partial deletion of a gene encoding at least one polypeptide in the microbial cell, or for effecting the inactivation of a gene encoding the at least one polypeptide in the microbial cell, where the at least one polypeptide is a regulator of transcription that controls the expression of one or more proteases; and optionally further comprising
   c. a vector comprising a nucleotide sequence coding for a compound of interest, wherein the nucleotide sequence is operably linked to a promoter.
102.A kit comprising:
   a. a modified microbial host cell, wherein microbial host cell has been modified to adversely affects the production, stability and/or function of at least one regulator of transcription that controls the expression of one or more proteases; and
   b. a vector comprising a nucleotide sequence coding for a compound of interest, wherein the nucleotide sequence is operably linked to a promoter.
103. The kit of embodiment 102, wherein the modified microbial host cell is a microbial host cell according to any one of embodiments 1 to 87.
104.A kit comprising:
   a. a vector for homologous recombination of a microbial cell, for example for effecting a full or partial deletion of at least one polypeptide encoded by the genome of the microbial cell, where the at least one polypeptide is a regulator of transcription that controls the expression of one or more proteases; and
   b. a vector comprising a nucleotide sequence coding for a compound of interest, wherein the nucleotide sequence is operably linked to a promoter.
105. The kit of any one of embodiments 101 to 104, wherein the kit further comprises instructions for use.
106. The kit of any one of embodiments 101 to 105, wherein the components of the kit are disposed separately in different containers.

1. A microbial host cell which is characterized by:
(a) having been modified and where this modification affects the production, stability and/or function of at least one polypeptide; and
(b) having a modulation in protease activity if compared with a parent microbial host cell which has not been modified and measured under the same conditions
wherein the at least one polypeptide:
(a) comprises a sequence selected from the group consisting of SEQ ID NOs: 1, 28, 33, 36, 58 and 59 or a polypeptide at least about 80%, at least about 85%, at least about 90%, at least about 95%, or at least about 98% identical thereto, or an ortholog thereof;
(b) is coded for by a genomic nucleotide sequence comprising a sequence selected from the group consisting of SEQ ID NOs: 2, 29, 34 and 37 or a polypeptide at least 80%, at least about 85%, at least about 90%, at least about 95%, or at least about 98% identical thereto, or an ortholog thereof;
(c) is coded for by a nucleotide sequence comprising a sequence selected from the group consisting of SEQ ID NOs: 3, 30, 35 and 38 or a polypeptide at least about 80%, at least about 85%, at least about 90%, at least about 95%, or at least about 98% identical thereto, or an ortholog thereof; or
(d) comprises a sequence having at least about 95% or 100% identity to the sequence of SEQ ID NO: 31;
wherein the at least one polypeptide is a promoter of transcription and has been modified to reduce its production, stability and/or function, and the modulation in protease activity is a reduction or deficiency in protease activity, and
wherein the microbial host cell further comprises at least one polynucleotide coding for a compound of interest.

2. The microbial host cell according to claim 1, wherein the modification is a genetic modification.

3. The microbial host cell according to claim 1, wherein the microbial host cell or a fermentation broth or cell culture medium containing said modified microbial host cell has at least about 40% less protease activity if compared with the intracellular environment of the parent microbial host cell which has not been modified or a fermentation broth or cell culture medium containing said parent microbial host cell which has not been modified and measured under the same conditions.

4. The microbial host cell according to claim 1, wherein the microbial host cell is a fungal cell, for example a filamentous fungal host cell, for example a filamentous fungus selected from the group consisting of Aspergillus, Acremonium, Myceliophthora, Thielavia Chrysosporium, Penicillium, Talaromyces, Rasamsonia, Fusarium or Trichoderma, preferably a species of Aspergillus niger, A. nidulans, Aspergillus awamori, Aspergillus foetidus, Aspergillus sojae, Aspergillus fumigatus, Aspergillus oryzae, Acremonium alabamense, Myceliophthora thermophila, Myceliophthora heterothallica, Thermothelomyces heterothallica, Thermothelomyces thermophilus, Thielavia terrestris, Chrysosporium lucknowense, Fusarium oxysporum, Rasamsonia emersonii, Talaromyces emersonii, Trichoderma reesei, Penicillium chrysogenum, Penicillium oxalicum and Neurospora crassa.

5. The microbial host cell according to claim 4 which is Trichoderma reesei, Myceliophthora heterothallica, Myceliophthora thermophilus or Aspergillus nidulans.

6. The microbial host cell of claim 1, wherein the compound of interest is an antibody or a functional fragment thereof, a carbohydrate binding domain, a heavy chain antibody or a functional fragment thereof, a single domain antibody, a heavy chain variable domain of an antibody or a functional fragment thereof, a heavy chain variable domain of a heavy chain antibody or a functional fragment thereof (VHH), a variable domain of camelid heavy chain antibody or a functional fragment thereof, a variable domain of a new antigen receptor (vNAR), a variable domain of shark new antigen receptor or a functional fragment thereof, a minibody, a nanobody, a nanoantibody, an affibody, an alphabody, a designed ankyrin-repeat domain, an anticalins, a knottins or an engineered CH2 domain.

7. The microbial host cell of claim 6, wherein the compound of interest is a heavy chain variable domain of a heavy chain antibody or a functional fragment thereof (VHH).

8. The microbial host cell of claim 7, wherein the VHH comprises:
(a) a CDR1 comprising or consisting of a sequence selected from the group consisting of SEQ ID NOs 45, 49 and 53;
(b) a CDR2 comprising or consisting of a sequence selected from the group consisting of SEQ ID NOs: 46, 50 and 54; and
(c) a CDR3 comprising or consisting of a sequence selected from the group consisting of SEQ ID NOs: 47, 51 and 55.

9. The microbial host cell of claim 7, wherein the VHH comprises:
(a) a CDR1 comprising or consisting of the sequence of SEQ ID NO: 45, a CDR2 comprising or consisting of the sequence of SEQ ID NO: 46 and a CDR3 comprising or consisting of the sequence of SEQ ID NO: 47;
(b) a CDR1 comprising or consisting of the sequence of SEQ ID NO: 49, a CDR2 comprising or consisting of the sequence of SEQ ID NO: 50 and a CDR3 comprising or consisting of the sequence of SEQ ID NO: 51 or
(c) a CDR1 comprising or consisting of the sequence of SEQ ID NO: 53, a CDR2 comprising or consisting of the sequence of SEQ ID NO: 54 and a CDR3 comprising or consisting of the sequence of SEQ ID NO: 55.

10. The microbial host cell of claim 7, wherein the VHH comprises or consists of a sequence selected from the group consisting of SEQ ID NOs: 43, 44, 48, 52, 56 and 57.

11. A method of producing a microbial host cell according to claim 1 comprising the steps of:
(a) providing a parent microbial host cell; and
(b) modifying the parent microbial host cell, wherein the modification affects the production, stability and/or function of the at least one polypeptide.

12. The method of claim 11, wherein the step of modifying the parent microbial host cell comprises targeting the at least one polypeptide, its corresponding chromosomal gene and/or its corresponding mRNA by anti-sense techniques, RNAi techniques, CRISPR techniques, a small molecule inhibitor, an antibody, an antibody fragment or a combination thereof.

13. The method of claim 11, wherein the method further comprises inserting the polynucleotide coding for a compound of interest into the microbial host cell.

14. A method for the production of a compound of interest comprising:
(a) providing a microbial host cell according to claim 1, wherein the microbial host cell is capable of expressing the compound of interest;
(b) culturing said microbial host cell under conditions conducive to the expression of a compound of interest; and
(c) optionally isolating a compound of interest from the culture medium.

15. Use of a modified microbial host cell for the production of a compound of interest, wherein the microbial host cell is characterized by
(a) having been modified and where this modification affects the production, stability and/or function of at least one polypeptide;
(b) having a reduction or deficiency in protease activity if compared with a parent microbial host cell which has not been modified and is measured under the same conditions; and
(c) comprising at least one polynucleotide coding for the compound of interest, wherein the microbial host cell is a microbial host cell according to claim 1.

16. A kit:
(a) comprising:
(i) a microbial cell; and
(ii) a vector for homologous recombination, for example for effecting a full or partial deletion of a gene encoding at least one polypeptide in the microbial cell, or for effecting the inactivation of a gene encoding the at least one polypeptide in the microbial cell, where the at least one polypeptide is a regulator of transcription that controls the expression of one or more proteases; and optionally further comprising
(iii) a vector comprising a nucleotide sequence coding for a compound of interest, wherein the nucleotide sequence is operably linked to a promoter;
(b) or comprising:
(i) a modified microbial host cell, wherein microbial host cell has been modified to adversely affect the production, stability and/or function of at least one regulator of transcription that controls the expression of one or more proteases, optionally wherein the modified microbial host cell is a microbial host cell according to claim 1; and
(ii) a vector comprising a nucleotide sequence coding for a compound of interest, wherein the nucleotide sequence is operably linked to a promoter;
(c) or comprising:
(i) a vector for homologous recombination of a microbial cell, for example for effecting a full or partial deletion of at least one polypeptide encoded by the genome of the microbial cell, where the at least one polypeptide is a regulator of transcription that controls the expression of one or more proteases; and
(ii) a vector comprising a nucleotide sequence coding for a compound of interest, wherein the nucleotide sequence is operably linked to a promoter.

17. The kit of claim 16, wherein the kit further comprises instructions for use and/or wherein the components of the kit are disposed separately in different containers.

18. The microbial host cell of claim 5, wherein the compound of interest is a heavy chain variable domain of a heavy chain antibody or a functional fragment thereof (VHH).

19. The method of claim 13, wherein the microbial host cell is Trichoderma reesei, Myceliophthora heterothallica, Myceliophthora thermophilus or Aspergillus nidulans, and wherein the compound of interest is a heavy chain variable domain of a heavy chain antibody or a functional fragment thereof (VHH).

20. The method of claim 14, wherein the microbial host cell is Trichoderma reesei, Myceliophthora heterothallica, Myceliophthora thermophilus or Aspergillus nidulans, and wherein the compound of interest is a heavy chain variable domain of a heavy chain antibody or a functional fragment thereof (VHH).

21. The use of claim 15, wherein the microbial host cell is Trichoderma reesei, Myceliophthora heterothallica, Myceliophthora thermophilus or Aspergillus nidulans, and wherein the compound of interest is a heavy chain variable domain of a heavy chain antibody or a functional fragment thereof (VHH).

22. The kit of claim 16, wherein the microbial host cell is Trichoderma reesei, Myceliophthora heterothallica, Myceliophthora thermophilus or Aspergillus nidulans, and wherein the compound of interest is a heavy chain variable domain of a heavy chain antibody or a functional fragment thereof (VHH).
